SEQUENCE LISTING (116 sequences)

SEQ ID NO: 1 - DNA, 220 bp, Artificial sequence
  L-DNA nucleic acid sequence
gtaaaacgacggccagttcgcgcgtttcggtgatgacggtgaaaaccattacaataacgtactgcatccagagttactagatgaaccatatgtacacttgacgttccatcagcctaatcacttcgatgatcctgcgttagactatgtcaagcagtcattagatcagctacctatgacatatgtaccagatgatcactagtatcgtcatagctgtttcctg

SEQ ID NO: 2 - DNA, 220 bp, Artificial sequence
  L-DNA nucleic acid sequence
gtaaaacgacggccagttctgacacatgcagctcccggagacggtcaattacccgatcgagccagtcgatcacttcgatgatcacgtagcgtctgctgatgacttcacgtatcagtctaatctacacgactcattcaacgtgaatcgactcaagttgccatcagacttagatcagctagatctgccatatgatctagtcgagtgtcatagctgtttcctg

SEQ ID NO: 3 - DNA, 220 bp, Artificial sequence
  L-DNA nucleic acid sequence
gtaaaacgacggccagtcagcttgtctgtaagcggatgccgggagcaattagacattgatgcatccagtcgacgtacgatatgatcagcacttagatctagatgtactagatgatcagttcaatctgcagctcaatgcagactagcagttactccatcacttcgatgatccagagttactagactagcactcatactagcgtcgtcatagctgtttcctg

SEQ ID NO: 4 - DNA, 220 bp, Artificial sequence
  L-DNA nucleic acid sequence
gtaaaacgacggccagtgacaagcccgtcagggcgcgtcagcgggtcattaggatgatcatgtgaatgctgatgtacacttagatcagtctaatcacgtgatgaatctgaagcgtcagctacgatatcgtaatgagctacgattagatccgaagtcattgatgcatcacgtagtagcatctgatgatcacttcgatgatcagtgtcatagctgtttcctg

SEQ ID NO: 5 - DNA, 220 bp, Artificial sequence
  L-DNA nucleic acid sequence
gtaaaacgacggccagtttggcgggtgtcggggctggcttaactatgatttaagacgacagttagagcactatgatcacgtagcgtctgctgatgacttcacgttctatcatagacatgtcatcgacggactagatcacgatctacatgcgaatccgaagttcatgatgcatccgaagtcattgatgcatcgactcaatgtaggtcatagctgtttcctg

SEQ ID NO: 6 - DNA, 220 bp, Artificial sequence
  L-DNA nucleic acid sequence
gtaaaacgacggccagtcggcatcagagcagattgtactgagagtgcatttatatgtacactatcagcacttagatgtgactaatcactagtatcagtcattcaatcgtcagcatgcgatctatcatacgatcgagtatccagagtcattgatgcatcctaagttcaatgtagatgatgatcacttcgatgatcagttcagatgtcatagctgtttcctg

SEQ ID NO: 7 - DNA, 220 bp, Artificial sequence
  L-DNA nucleic acid sequence
gtaaaacgacggccagtaccatatgcggtgtgaaataccgcacagatattttaacgtacagctagacgactagcagttacatcagtctaatctgaagcgtcagctacgatatcacttcgagctacgattagatcagcctaatccagatgtgatgacattgaagttagatgtccatctcaagcgattcgactatcacgtagatcgtcatagctgtttcctg

SEQ ID NO: 8 - DNA, 220 bp, Artificial sequence
  L-DNA nucleic acid sequence
gtaaaacgacggccagtgcgtaaggagaaaataccgcatcaggcgtgattttcagcactatcagctagtccatcgtaatgcagacgctgatgatctgaatgctaactcacatcagcctaatcacttcgatgatcacgtgagtacatctgatgtacatcagtctaatcacttcgatgatcgtatgaagtagttgctccatctacgtcatagctgtttcctg

SEQ ID NO: 9 - DNA, 220 bp, Artificial sequence
  L-DNA nucleic acid sequence
gtaaaacgacggccagtattcgccattcaggctgcgcaactgttgggattttgagtcgaatctgaatgctaacttccatcgtaatgcagacgctgatgatctcaagcgattcgacttggatcataacttcgatgtagatgatcacgtcaatgatcctgcgttagactatgtcaagcatgtagatccgatcgagccagtcgatcgtcatagctgtttcctg

SEQ ID NO: 10 - DNA, 220 bp, Artificial sequence
  L-DNA nucleic acid sequence
gtaaaacgacggccagtaagggcgatcggtgcgggcctcttcgctatacaattctacattcatacagctagtcgatcctgcatcagtcgatccgaagttcagcaatcctaagttcaatcacttcgatgatcctacatactcattcaatgtccatcacgtactgcatctgcatgctgacgtactgcatctcgatgtaccagatggtcatagctgtttcctg

SEQ ID NO: 11 - DNA, 220 bp, Artificial sequence
  L-DNA nucleic acid sequence
gtaaaacgacggccagttacgccagctggcgaaagggggatgtgctgacaacactaagttcaacttcgatcacttcgatgatcctgagttagactatctagatgtcaagcagtcattagatccagagttactagagctgcatgtcaacgactagcagttacatcctatcaagtctgatctagcagagcatgtaccagatgtctgtcatagctgtttcctg

SEQ ID NO: 12 - DNA, 100 bp, Artificial sequence
  DNA Barcode nucleic acid sequence
gtaaaacgacggccagtatatgaagtactcattagatcatagacagttactgctccatcatagtaatgagcaatagctacgatgtcatagctgtttcctg

SEQ ID NO: 13 - DNA, 90 bp, Artificial sequence
  short synthetic oligo nucleic acid sequence
tttgttggagagtttgatcctggctcagggtgaacgctggcggcgtgcctaagacatgcaagtcgtgcgggccgcggggttttactccgt

SEQ ID NO: 14 - DNA, 90 bp, Artificial sequence
  short synthetic oligo nucleic acid sequence
tttccccgggttgtccccctcttccgggtaggtcacccacgcgttactcacccgtccgccgctgaccacggagtaaaaccccgcggcccg

SEQ ID NO: 15 - DNA, 90 bp, Artificial sequence
  short synthetic oligo nucleic acid sequence
ggaagagggggacaacccggggaaactcgggctaatcccccatgtggacccgccccttggggtgtgtccaaagggctttgcccgcttccg

SEQ ID NO: 16 - DNA, 90 bp, Artificial sequence
  short synthetic oligo nucleic acid sequence
cggctacccgtcgtcgccttggtgggccattaccccaccaactagctgatgggacgcgggcccatccggaagcgggcaaagccctttgga

SEQ ID NO: 17 - DNA, 90 bp, Artificial sequence
  short synthetic oligo nucleic acid sequence
aaggcgacgacgggtagccggtctgagaggatggccggccacaggggcactgagacacgggccccactcctacgggaggcagcagttagg

SEQ ID NO: 18 - DNA, 90 bp, Artificial sequence
  short synthetic oligo nucleic acid sequence
accccgaagggcttcttcctccaagcggcgtcgctccgtcaggcttgcgcccattgcggaagattcctaactgctgcctcccgtaggagt

SEQ ID NO: 19 - DNA, 90 bp, Artificial sequence
  short synthetic oligo nucleic acid sequence
cttggaggaagaagcccttcggggtgtaaactcctgaacccgggacgaaacccccgacgaggggactgacggtaccggggtaatagcgcc

SEQ ID NO: 20 - DNA, 90 bp, Artificial sequence
  short synthetic oligo nucleic acid sequence
acgcccagtgaatccgggtaacgctcgcgccctccgtattaccgcggctgctggcacggagttggccggcgctattaccccggtaccgtc

SEQ ID NO: 21 - DNA, 90 bp, Artificial sequence
  short synthetic oligo nucleic acid sequence
gcgttacccggattcactgggcgtaaagggcgtgtaggcggcctggggcgtcccatgtgaaagaccacggctcaaccgtgggggagcgtg

SEQ ID NO: 22 - DNA, 90 bp, Artificial sequence
  short synthetic oligo nucleic acid sequence
tatctgcgcatttcaccgctactccgggaattccaccaccctctcccaccgtctagcctgagcgtatcccacgctcccccacggttgagc

SEQ ID NO: 23 - DNA, 90 bp, Artificial sequence
  short synthetic oligo nucleic acid sequence
aattcccggagtagcggtgaaatgcgcagataccgggaggaacgccgatggcgaaggcagccacctggtccacccgtgacgctgaggcgc

SEQ ID NO: 24 - DNA, 90 bp, Artificial sequence
  short synthetic oligo nucleic acid sequence
agacctagcgcgcatcgtttagggcgtggactacccgggtatctaatccggtttgctccccacgctttcgcgcctcagcgtcacgggtgg

SEQ ID NO: 25 - DNA, 90 bp, Artificial sequence
  short synthetic oligo nucleic acid sequence
ccctaaacgatgcgcgctaggtctctgggtctcctgggggccgaagctaacgcgttaagcgcgccgcctggggagtacggccgcaaggct

SEQ ID NO: 26 - DNA, 90 bp, Artificial sequence
  short synthetic oligo nucleic acid sequence
ttcgcgttgcttcgaattaaaccacatgctccaccgcttgtgcgggcccccgtcaattcctttgagtttcagccttgcggccgtactccc

SEQ ID NO: 27 - DNA, 90 bp, Artificial sequence
  short synthetic oligo nucleic acid sequence
ggagcatgtggtttaattcgaagcaacgcgaagaaccttaccaggccttgacatgctagggaacccgggtgaaagcctggggtgccccgc

SEQ ID NO: 28 - DNA, 90 bp, Artificial sequence
  short synthetic oligo nucleic acid sequence
ggacttaacccaacacctcacggcacgagctgacgacggccatgcagcacctgtgctagggctcccctcgcggggcaccccaggctttca

SEQ ID NO: 29 - DNA, 90 bp, Artificial sequence
  short synthetic oligo nucleic acid sequence
cgtgccgtgaggtgttgggttaagtcccgcaacgagcgcaacccccgccgttagttgccagcggttcggccgggcactctaacgggactg

SEQ ID NO: 30 - DNA, 90 bp, Artificial sequence
  short synthetic oligo nucleic acid sequence
tgtgtcgcccaggccgtaagggccatgctgaccagacgtcgtcccctccttcctcccgctttcgcgggcagtcccgttagagtgcccggc

SEQ ID NO: 31 - DNA, 90 bp, Artificial sequence
  short synthetic oligo nucleic acid sequence
ggcccttacggcctgggcgacacacgtgctacaatgcccactacaaagcgatgccacccggcaacggggagctaatcgcaaaaaggtggg

SEQ ID NO: 32 - DNA, 90 bp, Artificial sequence
  short synthetic oligo nucleic acid sequence
gatccgcgattactagcgattccggcttcatggggtcgggttgcagaccccaatccgaactgggcccacctttttgcgattagctccccg

SEQ ID NO: 33 - DNA, 90 bp, Artificial sequence
  short synthetic oligo nucleic acid sequence
gccggaatcgctagtaatcgcggatcagccatgccgcggtgaatacgttcccgggccttgtacacaccgcccgtcacgccatgggagcgg

SEQ ID NO: 34 - DNA, 90 bp, Artificial sequence
  short synthetic oligo nucleic acid sequence
cgacttcgccccagtcacgggccctaccctcggcgcctgcccgtaggctcccggcgacttcgggtagagcccgctcccatggcgtgacgg

SEQ ID NO: 35 - DNA, 54 bp, Artificial sequence
  short synthetic oligo nucleic acid sequence
ccgcaccttccggtacagctaccttgttacgacttcgccccagtcacgggccct

SEQ ID NO: 36 - DNA, 17 bp, Artificial sequence
  Single strand DNA oligonucleotide
gtaaaacgacggccagt

SEQ ID NO: 37 - DNA, 17 bp, Artificial sequence
  Single strand DNA oligonucleotide
caggaaacagctatgac

SEQ ID NO: 38 - DNA, 54 bp, Artificial sequence
  short synthetic oligo nucleic acid sequence
gtgctgcaaggcgattaattaggtatacaaccagaaccagattaagattgtata

SEQ ID NO: 39 - DNA, 69 bp, Artificial sequence
  short synthetic oligo nucleic acid sequence
ctatgactgttaacctatacaatcttaatctggttctggttgtatacctaattaatcgccttgcagcac

SEQ ID NO: 40 - DNA, 113 bp, Artificial sequence
  short synthetic D-/L- chimeric oligo nucleic acid sequence
ggttaacagtcatagctgtttcctggtaaaacgacggccagtattaccttaacaacctataccacatataccaggttcagattctataggttcacagtcatagctgtttcctg

SEQ ID NO: 41 - DNA, 98 bp, Artificial sequence
  short synthetic D-/L- chimeric oligo nucleic acid sequence
caggaaacagctatgactgtgaacctatagaatctgaacctggtatatgtggtataggttgttaaggtaatactggccgtcgttttaccaggaaacag

SEQ ID NO: 42 - DNA, 17 bp, Artificial sequence
  Single strand DNA oligonucleotide
gtgctgcaaggcgatta

SEQ ID NO: 43 - DNA, 17 bp, Artificial sequence
  Single strand DNA oligonucleotide
caggaaacagctatgac

SEQ ID NO: 44 - DNA, 17 bp, Artificial sequence
  Single strand L-DNA oligonucleotide
gtaaaacgacggccagt

SEQ ID NO: 45 - DNA, 17 bp, Artificial sequence
  Single strand L-DNA oligonucleotide
caggaaacagctatgac

SEQ ID NO: 46 - DNA, 167 bp, Artificial sequence
  D-/L- chimeric DNA nucleic acid sequence
gtgctgcaaggcgattaattaggtatacaaccagaaccagattaagattgtataggttaacagtcatagctgtttcctggtaaaacgacggccagtattaccttaacaacctataccacatataccaggttcagattctataggttcacagtcatagctgtttcctg

SEQ ID NO: 47 - protein, 775 aa, Artificial sequence
  Pfu DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 48 - protein, 775 aa, Artificial sequence
  mutant version of the Pfu DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVRAHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLAAVYEAIFGKPKEKVYADEIAKAWESGENLERVAKYSMEDAGATYELGKEFLPMEIQLSRLVGQPLWDVSRSSTGNLVEWFLLRKAYERNELAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYADTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 49 - protein, 775 aa, Artificial sequence
  Pfu-5m-55I amino acid sequence
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVRAHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLAAVYEAIFGKPKEKVYADEIAKAWESGENLERVAKYSMEDAGATYELGKEFLPMEIQLSRLVGQPLWDVSRSSTGNLVEWFLLRKAYERNELAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKKLLDYRQKAIKLLANSFYGYYGYAKARWYCKECAESVTAWGRKYIELVWKELEEKFGFKVLYADTDGLYATIPGGESEEVKKKALEFVKYINSKLPGLLELEYEGFYKRGFFVTKKRYAVVDEEGKVTTRGLEVVRRDWSEAAKETQARVLETLLKHGDVEEAVRVVKEVTQKLANYEVPPEKLAIYEQITRPLHEYKATGPHVAVAKKLAAKGVKIKPGMVIGYIVLRGDGPISNRAYLAEEYDPKKHKYDAEYYVENQVLPAVLRVLEGFGYRKEDLRYQKTRQVGLTSWLNPKKS

SEQ ID NO: 50 - protein, 775 aa, Artificial sequence
  Pfu-5m-46I amino acid sequence
MILDVDYITEEGKPVIRLFKKENGKFKIEHDRTFRPYFYALLRDDSKIEEVKKITGERHGKVVRVVDVEKVEKKFLGKPVTVWKLYLEHPQDVPTIREKVRAHPAVVDIFEYDIPFAKRYLIDKGLVPMEGEEELKMLAFDIETLYHEGEEFGKGPILMISYADENEAKVATWKNVDLPYVEVVSSEREMVKRFLRVVREKDPDVLVTYNGDSFDFPYLAKRAEKLGVKLTLGRDGSEPKMQRMGDMTAVEVKGRVHFDLYHVATRTLNLPTYTLAAVYEAVFGKPKEKVYADEIAKAWESGENLERVAKYSMEDAGATYELGKEFLPMEAQLSRLVGQPLWDVSRSSTGNLVEWFLLRKAYERNELAPNKPSEEEYQRRLRESYTGGFVKEPEKGLWENVVYLDFRALYPSIIITHNVSPDTLNLEGCKNYDVAPQVGHKFCKDFPGFIPSLLGHLLEERQKIKTKMKETQDPIEKILLDYRQKAIKLLANSFYGYYGYAKARWYCKECAESVTAWGRKYIELVWKELEEKFGFKVLYADTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 51 - protein, 775 aa, Artificial sequence
  mutant version of the Pfu DNA polymerase
MILDVDYITEEGKPVIRLFKKENGKFKIEHDRTFRPYFYALLRDDSKIEEVKKITGERHGKVVRVVDVEKVEKKFLGKPVTVWKLYLEHPQDVPTIREKVRAHPAVVDIFEYDIPFAKRYLIDKGLVPMEGEEELKMLAFDIETLYHEGEEFGKGPILMISYADENEAKVATWKNVDLPYVEVVSSEREMVKRFLRVVREKDPDVLVTYNGDSFDFPYLAKRAEKLGVKLTLGRDGSEPKMQRMGDMTAVEVKGRVHFDLYHVATRTLNLPTYTLAAVYEAVFGKPKEKVYADEIAKAWESGENLERVAKYSMEDAGATYELGKEFLPMEAQLSRLVGQPLWDVSRSSTGNLVEWFLLRKAYERNELAPNKPSEEEYQRRLRESYTGGFVKEPEKGLWENVVYLDFRALYPSIIITHNVSPDTLNLEGCKNYDVAPQVGHKFCKDFPGFIPSLLGHLLEERQKIKTKMKETQDPIEKKLLDYRQKAIKLLANSFYGYYGYAKARWYCKECAESVTAWGRKYIELVWKELEEKFGFKVLYADTDGLYATIPGGESEEVKKKALEFVKYINSKLPGLLELEYEGFYKRGFFVTKKRYAVVDEEGKVTTRGLEVVRRDWSEAAKETQARVLETLLKHGDVEEAVRVVKEVTQKLANYEVPPEKLAIYEQITRPLHEYKATGPHVAVAKKLAAKGVKIKPGMVIGYIVLRGDGPISNRAYLAEEYDPKKHKYDAEYYVENQVLPAVLRVLEGFGYRKEDLRYQKTRQVGLTSWLNPKKS

SEQ ID NO: 52 - protein, 775 aa, Artificial sequence
  mutant version of the Pfu DNA polymerase
MVLDVDYLTEEGKPVVRLFKKENGKFKVEHDRTFRPYFYALLRDDSKLEEVKKVTGERHGKVVRVVDVEKVEKKFLGKPVTVWKLYLEHPQDVPTLREKVRAHPAVVDVFEYDVPFAKRYLVDKGLVPMEGEEELKMLAFDVETLYHEGEEFGKGPVLMVSYADENEAKVATWKNVDLPYVEVVSSEREMVKRFLRVVREKDPDVLVTYNGDSFDFPYLAKRAEKLGVKLTLGRDGSEPKMQRMGDMTAVEVKGRVHFDLYHVATRTLNLPTYTLAAVYEAVFGKPKEKVYADEVAKAWESGENLERVAKYSMEDAGATYELGKEFLPMEAQLSRLVGQPLWDVSRSSTGNLVEWFLLRKAYERNELAPNKPSEEEYQRRLRESYTGGFVKEPEKGLWENVVYLDFRALYPSVVVTHNVSPDTLNLEGCKNYDVAPQVGHKFCKDFPGFVPSLLGHLLEERQKVKTKMKETQDPVEKKLLDYRQKAVKLLANSFYGYYGYAKARWYCKECAESVTAWGRKYVELVWKELEEKFGFKVLYADTDGLYATVPGGESEEVKKKALEFVKYVNSKLPGLLELEYEGFYKRGFFVTKKRYAVVDEEGKVTTRGLEVVRRDWSEAAKETQARVLETLLKHGDVEEAVRVVKEVTQKLANYEVPPEKLAVYEQVTRPLHEYKATGPHVAVAKKLAAKGVKVKPGMVVGYVVLRGDGPVSNRAYLAEEYDPKKHKYDAEYYVENQVLPAVLRVLEGFGYRKEDLRYQKTRQVGLTSWLNPKKS

SEQ ID NO: 53 - protein, 774 aa, Artificial sequence
  KOD1 polymerase
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYEEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYERGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 54 - protein, 773 aa, Artificial sequence
  Tgo polymerase
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 55 - protein, 775 aa, Artificial sequence
  9 degrees N-7 polymerase amino acid sequence
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 56 - protein, 773 aa, Artificial sequence
  Tok polymerase
MILDADYITEDGKPVIRVFKKEKGEFKIDYDRDFEPYIYALLKDDSAIEDIKKITAERHGTTVRVTRAERVKKKFLGRPVEVWKLYFTHPQDVPAIRDKIREHPAVVDIYEYDIPFAKRYLIDRGLIPMEGDEELRMLAFDIETLYHEGEEFGEGPILMISYADEEGARVITWKNIDLPYVESVSTEKEMIKRFLKVIQEKDPDVLITYNGDNFDFAYLKKRSEMLGVKFILGRDGSEPKIQRMGDRFAVEVKGRIHFDLYPVIRRTINLPTYTLETVYEPVFGQPKEKVYAEEIARAWESGEGLERVARYSMEDAKATYELGKEFFPMEAQLSRLVGQSLWDVSRSSTGNLVEWFLLRKAYERNDVAPNKPDERELARRTESYAGGYVKEPEKGLWENIVYLDYKSLYPSIIITHNVSPDTLNREGCREYDVAPQVGHRFCKDFPGFIPSLLGDLLEERQKVKKKMKATVDPIERKLLDYRQRAIKILANSYYGYYAYANARWYCRECAESVTAWGRQYIETTMREIEEKFGFKVLYADTDGFFATIPGADAETVKNKAKEFLNYINPRLPGLLELEYEGFYRRGFFVTKKKYAVIDEEDKITTRGLEIVRRDWSEIAKETQARVLEAILKHGDVEEAVRIVKEVTEKLSRHEVPPEKLVIYEQITRDLRSYRATGPHVAVAKRLAARGIKIRPGTVISYIVLKGPGRVGDRAIPFDEFDPAKHRYDAEYYIENQVLPAVERILRAFGYRKEDLRYQKTKQAGLGAWLKPKT

SEQ ID NO: 57 - protein, 473 aa, Artificial sequence
  Pfu DNA polymerase N fragment
HHHHHHMILDVDYITEEGKPVIRLFKKENGKFKIEHDRTFRPYFYALLRDDSKIEEVKKITGERHGKVVRVVDVEKVEKKFLGKPVTVWKLYLEHPQDVPTIREKVRAHPAVVDIFEYDIPFAKRYLIDKGLVPMEGEEELKMLAFDIETLYHEGEEFGKGPILMISYADENEAKVATWKNVDLPYVEVVSSEREMVKRFLRVVREKDPDVLVTYNGDSFDFPYLAKRAEKLGVKLTLGRDGSEPKMQRMGDMTAVEVKGRVHFDLYHVATRTLNLPTYTLAAVYEAVFGKPKEKVYADEIAKAWESGENLERVAKYSMEDAGATYELGKEFLPMEAQLSRLVGQPLWDVSRSSTGNLVEWFLLRKAYERNELAPNKPSEEEYQRRLRESYTGGFVKEPEKGLWENVVYLDFRALYPSIIITHNVSPDTLNLEGCKNYDVAPQVGHKFCKDFPGFIPSLLGHLLEERQKIKTK

SEQ ID NO: 58 - protein, 45 aa, Artificial sequence
  Pfu DNA polymerase N fragment
HHHHHHMILDVDYITEEGKPVIRLFKKENGKFKIEHDRTFRPYFY

SEQ ID NO: 59 - protein, 62 aa, Artificial sequence
  Pfu DNA polymerase N fragment
CLLRDDSKIEEVKKITGERHGKVVRVVDVEKVEKKFLGKPVTVWKLYLEHPQDVPTIREKVR

SEQ ID NO: 60 - protein, 60 aa, Artificial sequence
  Pfu DNA polymerase N fragment
  MISC_FEATURE (1)..(1): N-terminal Trifluoroacetyl thiazolidine-4- caboxylic acid (Tfa-Thz) conjugated
HPAVVDIFEYDIPFAKRYLIDKGLVPMEGEEELKMLAFDIETLYHEGEEFGKGPILMISY

SEQ ID NO: 61 - protein, 59 aa, Artificial sequence
  Pfu DNA polymerase N fragment
  MISC_FEATURE (1)..(1): N-terminal Trifluoroacetyl thiazolidine-4- caboxylic acid (Tfa-Thz) conjugated
DENEAKVATWKNVDLPYVEVVSSEREMVKRFLRVVREKDPDVLVTYNGDSFDFPYLAKR

SEQ ID NO: 62 - protein, 53 aa, Artificial sequence
  Pfu DNA polymerase N fragment
CEKLGVKLTLGRDGSEPKMQRMGDMTAVEVKGRVHFDLYHVATRTLNLPTYTL

SEQ ID NO: 63 - protein, 41 aa, Artificial sequence
  Pfu DNA polymerase N fragment
  MISC_FEATURE (1)..(1): N-terminal Trifluoroacetyl thiazolidine-4- caboxylic acid (Tfa-Thz) conjugated
AVYEAVFGKPKEKVYADEIAKAWESGENLERVAKYSMEDAG

SEQ ID NO: 64 - protein, 49 aa, Artificial sequence
  Pfu DNA polymerase N fragment
  MISC_FEATURE (1)..(1): N-terminal Trifluoroacetyl thiazolidine-4- caboxylic acid (Tfa-Thz) conjugated
TYELGKEFLPMEAQLSRLVGQPLWDVSRSSTGNLVEWFLLRKAYERNEL

SEQ ID NO: 65 - protein, 39 aa, Artificial sequence
  Pfu DNA polymerase N fragment
  MISC_FEATURE (1)..(1): N-terminal Trifluoroacetyl thiazolidine-4- caboxylic acid (Tfa-Thz) conjugated
PNKPSEEEYQRRLRESYTGGFVKEPEKGLWENVVYLDFR

SEQ ID NO: 66 - protein, 60 aa, Artificial sequence
  Pfu DNA polymerase N fragment
CLYPSIIITHNVSPDTLNLEGCKNYDVAPQVGHKFCKDFPGFIPSLLGHLLEERQKIKTK

SEQ ID NO: 67 - protein, 308 aa, Artificial sequence
  Pfu DNA polymerase C fragment
MKETQDPIEKKLLDYRQKAIKLLANSFYGYYGYAKARWYCKECAESVTAWGRKYIELVWKELEEKFGFKVLYADTDGLYATIPGGESEEVKKKALEFVKYINSKLPGLLELEYEGFYKRGFFVTKKRYAVVDEEGKVTTRGLEVVRRDWSEAAKETQARVLETLLKHGDVEEAVRVVKEVTQKLANYEVPPEKLAIYEQITRPLHEYKATGPHVAVAKKLAAKGVKIKPGMVIGYIVLRGDGPISNRAYLAEEYDPKKHKYDAEYYVENQVLPAVLRVLEGFGYRKEDLRYQKTRQVGLTSWLNPKKS

SEQ ID NO: 68 - protein, 33 aa, Artificial sequence
  Pfu DNA polymerase C fragment
MKETQDPIEKKLLDYRQKAIKLLANSFYGYYGY

SEQ ID NO: 69 - protein, 39 aa, Artificial sequence
  Pfu DNA polymerase C fragment
CKARWYCKECAESVTAWGRKYIELVWKELEEKFGFKVLY

SEQ ID NO: 70 - protein, 56 aa, Artificial sequence
  Pfu DNA polymerase C fragment
CDTDGLYATIPGGESEEVKKKALEFVKYINSKLPGLLELEYEGFYKRGFFVTKKRY

SEQ ID NO: 71 - protein, 55 aa, Artificial sequence
  Pfu DNA polymerase C fragment
  MISC_FEATURE (1)..(1): N-terminal Trifluoroacetyl thiazolidine-4- caboxylic acid (Tfa-Thz) conjugated
VVDEEGKVTTRGLEVVRRDWSEAAKETQARVLETLLKHGDVEEAVRVVKEVTQKL

SEQ ID NO: 72 - protein, 62 aa, Artificial sequence
  Pfu DNA polymerase C fragment
  MISC_FEATURE (1)..(1): N-terminal Trifluoroacetyl thiazolidine-4- caboxylic acid (Tfa-Thz) conjugated
NYEVPPEKLAIYEQITRPLHEYKATGPHVAVAKKLAAKGVKIKPGMVIGYIVLRGDGPISNR

SEQ ID NO: 73 - protein, 61 aa, Artificial sequence
  Pfu DNA polymerase C fragment
CYLAEEYDPKKHKYDAEYYVENQVLPAVLRVLEGFGYRKEDLRYQKTRQVGLTSWLNPKKS

SEQ ID NO: 74 - protein, 775 aa, Artificial sequence
  mutant version of the Pfu DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVRAHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALGPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 75 - protein, 775 aa, Artificial sequence
  mutant version of the Pfu DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDQPTIREKVRAHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALGPSIIITHNVSPDTLNLEGCKNYDIAPQVGHKFCKDIPGFIPSLLGHLLEERQKIKTKMKETQDPIEKILLDYRQKLIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 76 - protein, 775 aa, Artificial sequence
  mutant version of the Pfu DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDQPTIREKVRAHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALGPSIIITHNVSPDTLNLEGCKNYDIAPQVGHKFCKDIPGFIPSLLGHLLEERQKIKTKMKETQDPIEKILLDYRQKLIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYKQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 77 - protein, 682 aa, Artificial sequence
  mutant version of the Pfu DNA polymerase
PTIREKVRAHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFAFPYLAKRAEKLGIKLTIGRDGSEPKMQRIGDMTAVEVKGRIHFDLYHVITRTINLPTYTLAAVYEAIFGKPKEKVYADEIAKAWESGENLERVAKYSMEDAGATYELGKEFLPMEIQLSRLVGQPLWDVSRSSTGNLVEWFLLRKAYERNELAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLWANSFYGYYGYAKARWYCKECAESVTAWGRKYIELVWKELEEKFGFKVLYADTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 78 - protein, 63 aa, Artificial sequence
  sso7d structural domain amino acid sequence
ATVKFKYKGEEKEVDISKIKKVWRVGKMISFTYDEGGGKTGRGAVSEKDAPKELLQMLEKQKK

SEQ ID NO: 79 - protein, 844 aa, Artificial sequence
  Pfu DNA polymerase amino acid sequence
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVRAHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLAAVYEAIFGKPKEKVYADEIAKAWESGENLERVAKYSMEDAGATYELGKEFLPMEIQLSRLVGQPLWDVSRSSTGNLVEWFLLRKAYERNELAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYADTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKSGTGGGGATVKFKYKGEEKEVDISKIKKVWRVGKMISFTYDEGGGKTGRGAVSEKDAPKELLQMLEKQKK

SEQ ID NO: 80 - DNA, 1228 bp, Artificial Sequence
  pUC19 plasmid nucleic acid sequence
gcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacag

SEQ ID NO: 81 - DNA, 1523 bp, Artificial sequence
  DNA template coding for the bacterial 16S rRNA gene
taatacgactcactataggtttgttggagagtttgatcctggctcagggtgaacgctggcggcgtgcctaagacatgcaagtcgtgcgggccgcggggttttactccgtggtcagcggcggacgggtgagtaacgcgtgggtgacctacccggaagagggggacaacccggggaaactcgggctaatcccccatgtggacccgccccttggggtgtgtccaaagggctttgcccgcttccggatgggcccgcgtcccatcagctagttggtggggtaatggcccaccaaggcgacgacgggtagccggtctgagaggatggccggccacaggggcactgagacacgggccccactcctacgggaggcagcagttaggaatcttccgcaatgggcgcaagcctgacggagcgacgccgcttggaggaagaagcccttcggggtgtaaactcctgaacccgggacgaaacccccgacgaggggactgacggtaccggggtaatagcgccggccaactccgtgccagcagccgcggtaatacggagggcgcgagcgttacccggattcactgggcgtaaagggcgtgtaggcggcctggggcgtcccatgtgaaagaccacggctcaaccgtgggggagcgtgggatacgctcaggctagacggtgggagagggtggtggaattcccggagtagcggtgaaatgcgcagataccgggaggaacgccgatggcgaaggcagccacctggtccacccgtgacgctgaggcgcgaaagcgtggggagcaaaccggattagatacccgggtagtccacgccctaaacgatgcgcgctaggtctctgggtctcctgggggccgaagctaacgcgttaagcgcgccgcctggggagtacggccgcaaggctgaaactcaaaggaattgacgggggcccgcacaagcggtggagcatgtggtttaattcgaagcaacgcgaagaaccttaccaggccttgacatgctagggaacccgggtgaaagcctggggtgccccgcgaggggagccctagcacaggtgctgcatggccgtcgtcagctcgtgccgtgaggtgttgggttaagtcccgcaacgagcgcaacccccgccgttagttgccagcggttcggccgggcactctaacgggactgcccgcgaaagcgggaggaaggaggggacgacgtctggtcagcatggcccttacggcctgggcgacacacgtgctacaatgcccactacaaagcgatgccacccggcaacggggagctaatcgcaaaaaggtgggcccagttcggattggggtctgcaacccgaccccatgaagccggaatcgctagtaatcgcggatcagccatgccgcggtgaatacgttcccgggccttgtacacaccgcccgtcacgccatgggagcgggctctacccgaagtcgccgggagcctacgggcaggcgccgagggtagggcccgtgactggggcgaagtcgtaacaaggtagctgtaccggaaggtgcgg

SEQ ID NO: 82 - protein, 883 aa, Artificial sequence
  T7-WT amino acid sequence
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 83 - protein, 883 aa, Artificial sequence
  T7-37I (I6V, I14L, I74L, I82V, I109V, I117L, I141V, I219M, I244L, I281V, I320V, I322L, I330V, I367L)  amino acid sequence
MNTINVAKNDFSDLELAAIPFNTLADHYGERLAREQLALEHESYEMGEARFRKMFERQLKAGEVADNAAAKPLVTTLLPKMVARINDWFEEVKAKRGKRPTAFQFLQEVKPEAVAYLTIKTTLACLTSADNTTVQAVASAVGRAIEDEARFGRIRDLEAKHFKKNVEEQLNKRVGHVYKKAFMQVVEADMLSKGLLGGEAWSSWHKEDSMHVGVRCIEMLIESTGMVSLHRQNAGVVGQDSETLELAPEYAEAIATRAGALAGISPMFQPCVVPPKPWTGVTGGGYWANGRRPLALVRTHSKKALMRYEDVYMPEVYKAVNLAQNTAWKVNKKVLAVANVITKWKHCPVEDIPAIEREELPMKPEDLDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 84 - protein, 895 aa, Artificial sequence
  YenP amino acid sequence
MNILNHVARNDFSEMELAAIPYNTLSEHYGDRLAREQLALEHEAYELGEKRFLKMLDRQAQAGELADNAAAKPLVATLVPRMASRVTEWVDESFFVTDPETGEKKGKKGKRSVSQRILREVKPEVVAFVTIKCVLGGLTSTGSTVVQRLASSVGKALEEEMRFGRIRDLEQKHFKKNVQEQLEKRVGHVYKKAFLQVVEADMLSKGLMGGEAWSSWTAEDTIQVGVKCIELLIESTGLVELEREGHGIAGADSENVKLADEYMDIITKRAGALAGIAPMHQPCVVPPKPWTGVIGGGYWANGRKPLALVRTHSKKALLRYEDVYMPEVYKAVNIAQNTPWKINKKVLAVVNQVTKWQHCPVADVPAQEREELPQRPDDMDTNPEALKAWKKAASAVYRKDKARVSRRLSLEFMLAQANKFANHKAIWFPYNMDWRGRVYAVSMFNPQGNDMTKGLLTLAKGKPIGAEGFYWLKIHGANTAGVDKVTFPERIKFIEDNHANIMACAKSPLENTWWAEQDSPFCFLAFCFEYAGVQSHGLGYVCSLPLAFDGSCSGIQHFSAMLRDEVGGRAVNLLPSEEVQDIYGIVAQRVNEMLLEATVSGTADETETLVNKDTGEITERLKLGTAKLAAQWLAFGVTRKVTKRSVMTLAYGSKEFGFRQQVLEDTVQPALDNGQGLMFTQPNQAAGYMAKLIWEAVSVTVVAAVEAMNWLKASAKLLAAEVKDKKTDEVLRARCAVHWVTPDGFPVWQEYRKPVQSRLNLMFLGQFRLQPTINTNKDSGIDAHKQESGIAPNFVHSQDGNHLRMTVVHAHEAYGIESFALIHDSFGTIPADAANLFKAVRETMVSTYEENDVLADFYDQFADQLHESQLEKMPALPAKGSLNLQDILKSDFAFA

SEQ ID NO: 85 - protein, 893 aa, Artificial sequence
  phiEap amino acid sequence
MSLMNSIERNDFSDIELAAIPYNILSEHYGASLAKEQLALEHEAYELGEQRFLKTFERQMNNGELADNAAAKPLVLTLLPRLVSRINQWRHEQVYKLNDKGEEVLRGGKKTIMFNLTATLTAEAVALATIKTVLASLTSTNAVTIQEAGSAIGRAIEDEARFGRIRDLEEKHFKKNVKEQLEKRVGTVYKKAFMQVIEADMLSKGLMGGQAWASWSKEDSIHVGIRCIELLIEATGMVEVVRHGAGIAKLDSENLQLTKEYADLITKRAGALAGISPMFQPCVVPPKPWTEITGGGYWANGRRPLALVRTHGKKALMRYQDVYMPEVYKAINIAQSTPWKINQKVLKVANEIVNWKNCPVEDVPATERKELPVKPDDIDTNPEALKAWKKAASTIYRNDKARVSRRISMEFMLAQANKFANKKAIWFPYNMDWRGRVYAVPMFNPQGNDLTKGLLTLAKGKPIGLDGLYWLKIHGANCAGVDKVPFPERIKFIEENHDNIMQSAANPIEFDWWAKQDSPFCFLAFCFEYAGVEHHGLNYNCSLPLAFDGSCSGIQHFSAMLRDEIGGRAVNLLPSTEVQDIYRIVADKVNEILKVDAVNGTANEVELITDKTTGEIKERLKVGTSVMAQEWLAFGVTRKVTKRSVMTLAYGSKEFGFRQQILDDTVQPAIDNGYTGFTNGNQSAGYMAKLIWNAVSVTVVAAVEAMNWLKSAAKLLAAEVKDKKTKEVLRARCAVHWVTPDGFPVWQEYRKSKQVRLDLIFLGSIRLQPTVNTNKDTGIDAHKQESGIAPNFVHSQDGNHLRATVVHAHEKYGIESFALIHDSFGTIPADAANLFKAVRETMVETYESNDVLADFYEQFADQLHESQLEDMPALPKAGNLNLQDILKSDFAFA

SEQ ID NO: 86 - protein, 906 aa, Artificial sequence
  KpnP amino acid sequence
MNALNIARNDFSEIELAAIPYNILSEHYGDKLAREQLALEHEAYELGEQRFLKMLERQVKAGEFADNVAAKPLVLTLHPQLTKRIDDWKEEQANARGKKPRAYYPIKHGVASELAVNMGAEVLKEKRGVSSEAIALLTIKVVLGTLTDASKATIQQVSSQLGKALEDEARFGRIREQEAAYFKKNVADQLDKRVGHVYKKAFMQVVEADMISKGMLGGDNWSSWKTDEQMHVGTKLLELLIEGTGLVEMTKNKMADGSDDVTSMQMVQLAPAFVELLSKRAGALAGISPMYQPCVVPPKPWVETVGGGYWSVGRRPLALVRTHSKKALRRYEDVHMPEVYKAVNLAQNTPWKVNKKVLAVVNEIINWKHCPVGDVPAIEREELPPRPDDIDTNEVARKAWRKEAAAVYRKDKARQSRRLSMEFMVAQANKFANHKAIWFPYNMDWRGRVYAVSMFNPQGNDMTKGMLTLAKGKPIGLDGFYWLKIHGANCAGVDKVPFPERIKFIEENDANILASAADPLNNTWWTQQDSPFCFLAFCFEYAGVKHHGLNYNCSLPLAFDGSCSGIQHFSAMLRDSIGGRAVNLLPSDTVQDIYKIVADKVNEVLHQHVINGSQTVVEQIADKETGEFREKVTLGESVLAAQWLQYGVTRKVTKRSVMTLAYGSKEFGFRQQVLEDTIQPAIDNGEGLMFTHPNQAAGYMAKLIWDAVTVTVVAAVEAMNWLKSAAKLLAAEVKDKKTKEVLRKRCAIHWVTPDGFPVWQEYRKQNQARLKLVFLGQANVKMTYNTGKDSEIDAHKQESGIAPNFVHSQDGSHLRMTVVHANEVYGIDSFALIHDSFGTIPADAGNLFKAVRETMVKTYEDNDVIADFYDQFADQLHESQLDKMPAVPAKGDLNLRDILESDFAFA

SEQ ID NO: 87 - protein, 369 aa, Artificial sequence
  T7-split-N fragment amino acid sequence
HHHHHHMNTINVAKNDFSDLELAAIPFNTLADHYGERLAREQLALEHESYEMGEARFRKMFERQLKAGEVADNAAAKPLVTTLLPKMVARINDWFEEVKAKRGKRPTAFQFLQEVKPEAVAYLTIKTTLACLTSADNTTVQAVASAVGRAIEDEARFGRIRDLEAKHFKKNVEEQLNKRVGHVYKKAFMQVVEADMLSKGLLGGEAWSSWHKEDSMHVGVRCIEMLIESTGMVSLHRQNAGVVGQDSETLELAPEYAEAIATRAGALAGISPMFQPCVVPPKPWTGVTGGGYWANGRRPLALVRTHSKKALMRYEDVYMPEVYKAVNLAQNTAWKVNKKVLAVANVITKWKHCPVEDIPAIEREELPMK

SEQ ID NO: 88 - protein, 43 aa, Artificial sequence
  T7-N-1 amino acid sequence
HHHHHHMNTINVAKNDFSDLELAAIPFNTLADHYGERLAREQL

SEQ ID NO: 89 - protein, 32 aa, Artificial sequence
  T7-N-2 amino acid sequence
CLEHESYEMGEARFRKMFERQLKAGEVADNAA

SEQ ID NO: 90 - protein, 70 aa, Artificial sequence
  T7-N-3 amino acid sequence
CKPLVTTLLPKMVARINDWFEEVKAKRGKRPTAFQFLQEVKPEAVAYLTIKTTLACLTSADNTTVQAVAS

SEQ ID NO: 91 - protein, 76 aa, Artificial sequence
  T7-N-4 amino acid sequence
CVGRAIEDEARFGRIRDLEAKHFKKNVEEQLNKRVGHVYKKAFMQVVEADMLSKGLLGGEAWSSWHKEDSMHVGVR

SEQ ID NO: 92 - protein, 44 aa, Artificial sequence
  T7-N-5 amino acid sequence
CIEMLIESTGMVSLHRQNAGVVGQDSETLELAPEYAEAIATRAG

SEQ ID NO: 93 - protein, 34 aa, Artificial sequence
  T7-N-6 amino acid sequence
  MISC_FEATURE (1)..(1): N-terminal Trifluoroacetyl thiazolidine-4- caboxylic acid (Tfa-Thz) conjugated
LAGISPMFQPCVVPPKPWTGVTGGGYWANGRRPL

SEQ ID NO: 94 - protein, 69 aa, Artificial sequence
  T7-N-7 amino acid sequence
CLVRTHSKKALMRYEDVYMPEVYKAVNLAQNTAWKVNKKVLAVANVITKWKHCPVEDIPAIEREELPMK

SEQ ID NO: 95 - protein, 238 aa, Artificial sequence
  T7-split-M fragment amino acid sequence
PEDLDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDEN

SEQ ID NO: 96 - protein, 45 aa, Artificial sequence
  T7-M-1 amino acis sequence
PEDLDMNPEALTAWKRAAAAVYRKDKARKSRRISLEFMLEQANKF

SEQ ID NO: 97 - protein, 38 aa, Artificial sequence
  T7-M-2 amino acis sequence
CNHKAIWFPYNMDWRGRVYAVSMFNPQGNDMTKGLLTL

SEQ ID NO: 98 - protein, 44 aa, Artificial sequence
  T7-M-3 amino acis sequence
CKGKPIGKEGYYWLKIHGANCAGVDKVPFPERIKFIEENHENIM

SEQ ID NO: 99 - protein, 43 aa, Artificial sequence
  T7-M-4 amino acis sequence
  MISC_FEATURE (1)..(1): N-terminal Trifluoroacetyl thiazolidine-4- caboxylic acid (Tfa-Thz) conjugated
CAKSPLENTWWAEQDSPFCFLAFCFEYAGVQHHGLSYNCSLPL

SEQ ID NO: 100 - protein, 22 aa, Artificial sequence
  T7-M-5 amino acis sequence
  MISC_FEATURE (1)..(1): N-terminal Trifluoroacetyl thiazolidine-4- caboxylic acid (Tfa-Thz) conjugated
FDGSCSGIQHFSAMLRDEVGGR

SEQ ID NO: 101 - protein, 44 aa, Artificial sequence
  T7-M-6 amino acis sequence
CVNLLPSETVQDIYGIVAKKVNEILQADAINGTDNEVVTVTDEN

SEQ ID NO: 102 - protein, 282 aa, Artificial sequence
  T7-split-C fragment amino acid sequence
TGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 103 - protein, 72 aa, Artificial sequence
  T7-C-1 amino acid sequence
TGEISEKVKLGTKALAGQWLAYGVTRSVTKRSVMTLAYGSKEFGFRQQVLEDTIQPAIDSGKGLMFTQPNQA

SEQ ID NO: 104 - protein, 49 aa, Artificial sequence
  T7-C-2 amino acid sequence
CGYMAKLIWESVSVTVVAAVEAMNWLKSAAKLLAAEVKDKKTGEILRKR

SEQ ID NO: 105 - protein, 75 aa, Artificial sequence
  T7-C-3 amino acid sequence
CAVHWVTPDGFPVWQEYKKPIQTRLNLMFLGQFRLQPTINTNKDSEIDAHKQESGIAPNFVHSQDGSHLRKTVVW

SEQ ID NO: 106 - protein, 44 aa, Artificial sequence
  T7-C-4 amino acid sequence
  MISC_FEATURE (1)..(1): N-terminal Trifluoroacetyl thiazolidine-4- caboxylic acid (Tfa-Thz) conjugated
HEKYGIESFALIHDSFGTIPADAANLFKAVRETMVDTYESCDVL

SEQ ID NO: 107 - protein, 41 aa, Artificial sequence
  T7-C-5 amino acid sequence
CDFYDQFADQLHESQLDKMPALPAKGNLNLRDILESDFAFA

SEQ ID NO: 108 - DNA, 160 bp, Artificial sequence
  DNA template nucleic acid sequence
gatctcgatcccgcgaaattaatacgactcactataggtcccccgtgcccatagcggcgtggaaccacccgttcccattccgaacacggaagtgaaacgcgccagcgccgatggtactgggcgggcgaccgcctgggagagtaggtcggtgcgggggatt

SEQ ID NO: 109 - DNA, 1539 bp, Artificial sequence
  Tt 16S DNA template nucleic acid sequence
ctcgatcccgcgaaattaatacgactcactataggtttgttggagagtttgatcctggctcagggtgaacgctggcggcgtgcctaagacatgcaagtcgtgcgggccgcggggttttactccgtggtcagcggcggacgggtgagtaacgcgtgggtgacctacccggaagagggggacaacccggggaaactcgggctaatcccccatgtggacccgccccttggggtgtgtccaaagggctttgcccgcttccggatgggcccgcgtcccatcagctagttggtggggtaatggcccaccaaggcgacgacgggtagccggtctgagaggatggccggccacaggggcactgagacacgggccccactcctacgggaggcagcagttaggaatcttccgcaatgggcgcaagcctgacggagcgacgccgcttggaggaagaagcccttcggggtgtaaactcctgaacccgggacgaaacccccgacgaggggactgacggtaccggggtaatagcgccggccaactccgtgccagcagccgcggtaatacggagggcgcgagcgttacccggattcactgggcgtaaagggcgtgtaggcggcctggggcgtcccatgtgaaagaccacggctcaaccgtgggggagcgtgggatacgctcaggctagacggtgggagagggtggtggaattcccggagtagcggtgaaatgcgcagataccgggaggaacgccgatggcgaaggcagccacctggtccacccgtgacgctgaggcgcgaaagcgtggggagcaaaccggattagatacccgggtagtccacgccctaaacgatgcgcgctaggtctctgggtctcctgggggccgaagctaacgcgttaagcgcgccgcctggggagtacggccgcaaggctgaaactcaaaggaattgacgggggcccgcacaagcggtggagcatgtggtttaattcgaagcaacgcgaagaaccttaccaggccttgacatgctagggaacccgggtgaaagcctggggtgccccgcgaggggagccctagcacaggtgctgcatggccgtcgtcagctcgtgccgtgaggtgttgggttaagtcccgcaacgagcgcaacccccgccgttagttgccagcggttcggccgggcactctaacgggactgcccgcgaaagcgggaggaaggaggggacgacgtctggtcagcatggcccttacggcctgggcgacacacgtgctacaatgcccactacaaagcgatgccacccggcaacggggagctaatcgcaaaaaggtgggcccagttcggattggggtctgcaacccgaccccatgaagccggaatcgctagtaatcgcggatcagccatgccgcggtgaatacgttcccgggccttgtacacaccgcccgtcacgccatgggagcgggctctacccgaagtcgccgggagcctacgggcaggcgccgagggtagggcccgtgactggggcgaagtcgtaacaaggtagctgtaccggaaggtgcgg

SEQ ID NO: 110 - DNA, 113 bp, Artificial sequence
  tRNA(Ser) DNA template
tggcggagagagggggatttgaacccccggtagagttgcccctactccggttttcgagaccggtccgttcagccgctccggcatctctcctatagtgagtcgtattagaaccg

SEQ ID NO: 111 - DNA, 163 bp, Artificial Sequence
  L-guanine sensor DNA template
ctcgatcccgcgaaattaatacgactcactatagacgcgactgaatgaaatggtgaaggacgggtccagataatcgcgtggatatggcacgcaagtttctaccgggcaccgtaaatgtccgactcttgttgagtagagtgtgagctccgtaactagtcgcgtc

SEQ ID NO: 112 - DNA, 203 bp, Artificial Sequence
  DNA template of L- 38-6 ribozyme
ggactaatacgactcactattagtcattgccgcacaaagacaaatctcccctcagagcttgagaacatctacggatgcagaggagggggccttcggtggatcaattgtgcaccaccgttctcaacacgtacccgaacataaaaagacctgacaaaaaggcgatgttagacacgcacaggtgccatacccaacacatggctgac

SEQ ID NO: 113 - DNA, 133 bp, Artificial Sequence
  L- class I ligase DNA template
ggactaatacgactcactattgacaatgacaaaaaatcactattgttgagaacgttggcgttaaagccaccgggggctgcctcccctgcatccgaagatgttctcaagctctgagggcagatttgtcttttcc

SEQ ID NO: 114 - DNA, 182 bp, Artificial Sequence
  L- 38-6 ribozyme
agucauugccgcacaaagacaaaucuccccucagagcuugagaacaucuacggaugcagaggagggggccuucgguggaucaauugugcaccaccguucucaacacguacccgaacauaaaaagaccugacaaaaaggcgauguuagacacgcacaggugccauacccaacacauggcugac

SEQ ID NO: 115 - DNA, 20 bp, Artificial Sequence
  L- 5′-FAM-labelled primer
  misc_feature (1)..(1): FAM labelled
  misc_feature (1)..(1): FAM conjugated
ggaaaagacaaaucugcccu

SEQ ID NO: 116 - DNA, 112 bp, Artificial Sequence
  L- class I ligase template
gacaaugacaaaaaaucacuauuguugagaacguuggcguuaaagccaccgggggcugccuccccugcauccgaagauguucucaagcucugagggcagauuugucuuuucc

(19) United States
(12) Patent Application Publication
(10) Pub. No.: US 2023/0313156 A1
(43) Pub. Date: Oct. 5, 2023

(54) CHEMICAL SYNTHESIS OF LARGE AND MIRROR-IMAGE PROTEINS AND USES THEREOF

(71) Applicant: Tsinghua University, Beijing (CN)

(72) Inventors: Ting ZHU, Beijing (CN); Chuyao FAN, Beijing (CN); Qiang DENG, Beijing (CN); Yuan XU, Beijing (CN)

(73) Assignee: Tsinghua University, Beijing (CN)

(21) Appl. No.: 18/019,847

(22) PCT Filed: May 13, 2021
(86) PCT No.: PCT/IB2021/054106

§ 371 Date: Feb. 6, 2023

Related U.S. Application Data
(60) Provisional application No. 63/061,844, filed on Aug. 6, 2020.

(51) Int. Cl. C12N 9/12; C07K 1/02; C12P 19/34

(52) U.S. Cl. CPC C12N 9/1252; C07K 1/026; C12P 19/34; C12Y 207/07007; C12N 9/1247; C12Y 207/07006

(57) ABSTRACT

Provided herein is a general method for producing large (more than 400 aa long) D-amino acids proteins, also referred to as mirror image protein (with respect to their naturally occurring L-amino acids counterparts), including RNA/DNA manipulating enzymes, and uses thereof in a wide range of research, practical data storage and medicinal applications.

### RELATED APPLICATION

This application claims the benefit of priority of U.S. Provisional Patent Application No. 63/061,844 filed 6 Aug. 2020, the contents of which are incorporated herein by reference in their entirety.

### SEQUENCE LISTING STATEMENT

The ASCII file, entitled 87597_ST25.txt, created on May 6, 2021, comprising 180,286 bytes, submitted concurrently with the filing of this application is incorporated herein by reference.

### FIELD AND BACKGROUND OF THE INVENTION

The present invention, in some embodiments thereof, relates to biochemistry and more particularly, but not exclusively, to methods of total chemical synthesis of large proteins and their mirror-image counterparts, and uses thereof.

Proteins composed entirely of unnatural D-amino acids and the achiral amino acid glycine are mirror image forms of their native L-protein counterparts. Recent advances in chemical protein synthesis afford unique and facile synthetic access to domain sized mirror image D-proteins, enabling protein research to be conducted through “the looking glass” and in a way previously unattainable. D-Proteins can facilitate structure determination of their native L-forms that are difficult to crystallize (racemic Xray crystallography); D-proteins can serve as the bait for library screening to ultimately yield pharmacologically superior D-peptide/D-protein therapeutics (mirror-image phage display); D-proteins can also be used as a powerful mechanistic tool for probing molecular events in biology, drug discovery, and immunology.

The single-handedness of biological molecules has fascinated scientists and laymen alike since Pasteur's first painstaking separation of the enantiomorphic crystals of a tartrate salt more than 160 year ago. More recently, a number of theoretical and experimental investigations have helped to delineate models for how one enantiomer might have come to dominate over the other from what presumably was a racemic prebiotic world. Blackmond, D. G., [“The Origin of Biological Homochirality”, Cold Spring Harb Perspect Biol., 2010, 2(5), a002147] highlights mechanisms for enantioenrichment that include either chemical or physical processes, or a combination of both. One of the scientific driving force for such endeavors arises from an interest in understanding the origin of life, because the homochirality of biological molecules is a signature of life. Other motivations arise from practical and applied scientific interests, such as orthogonal biological tools that can offer nature-impervious molecular systems, e.g., for safe data storage.

On the nucleic acid front, phosphoramidate chemistry has enabled oligonucleotide (oligo) synthesis of up to about 150 nt for DNA and about 70 nt for RNA. On the protein front, a conjunction between solid-phase peptide synthesis (SPPS) and native chemical ligation (NCL) has yielded a powerful method that enabled the total chemical synthesis of various proteins (5, 14-20). Specifically, mirror-image genetic replication and transcription system have been realized based on the mirror-image version of the 174-aa African swine fever virus polymerase X (ASFV pol X) (5), followed by a more efficient and thermostable 352-aa Sulfolobus solfataricus P2 DNA polymerase IV (Dpo4) (17-19), leading to the realization of mirror-image polymerase chain reaction (MI-PCR), as well as mirror-image gene transcription and reverse transcription (21). In particular, with a mutant version of D-Dpo4, full-length 5S rRNA enzymatically transcribed at 120 nt, a feat that was otherwise too long to be chemically synthesized (21).

Mirror image proteins are powerful tools with a wide range of applications in structural biology, peptide/protein drug design, and mechanistic studies of biological processes. As chemical protein synthesis techniques become more robust and readily available to scientists from different disciplines, the huge potential of mirror image proteins in chemical, biological, and biomedical research will be fully unlocked. The two enabling technologies—native chemical ligation and mirror-image phage display are particularly attractive, and will have a profound impact on the discovery of novel classes of pharmacologically superior peptide and protein therapeutics for the treatment of a variety of human diseases.

The review “Mirror image proteins” [Zhao, L. and Lu, W., Current Opinion in Chemical Biology, 2014, 22, pp. 56-61] examines recent progress in the application of mirror image proteins to structural biology, drug discovery, and immunology.

Hartrampf, N. et al. [“Synthesis of proteins by automated flow chemistry”, Science, 2020, 368(6494), pp. 980-987] report highly efficient chemistry matched with an automated fast-flow instrument for the direct manufacturing of peptide chains up to 164 amino acids long over 327 consecutive reactions, wherein peptide chain elongation is complete in hours, as demonstrated by the chemical synthesis of nine different protein chains that represent enzymes, structural units, and regulatory factors. The researchers report that after purification and folding, the synthetic materials display biophysical and enzymatic properties comparable to the biologically expressed proteins, showing that high-fidelity automated flow chemistry, or automated fast-flow peptide synthesis (AFPS), is an alternative technology for producing single-domain proteins without a ribosome.

However, mirror image proteins remain restrained to relatively small proteins, whereas the synthesis of larger ones with more than about 400 amino acid (aa) residues are much harder to achieve mainly owing to the limited synthesis and ligation efficiencies of peptide segments. Although a recently developed automated fast-flow peptide synthesis (AFPS) technology is able to yield peptide chains more than three times longer than previously accessible by routine standard SPPS, the apparent lack of proper methodology to synthesize large mirror-image molecules have prohibitively constrained the development of mirror-image biology systems and their applications such as in information storage.

### SUMMARY OF THE INVENTION

Aspect so the present invention are drawn to methods of total chemical synthesis of relatively large proteins (longer than 400 aa) in both the L- and D-handedness of their amino-acid residues, and applications for D-amino acids proteins, prepared according to the methods disclosed herein. Large proteins are chemically synthesized without the involvement or presence of biochemical macromolecules, according to embodiments of the present invention, by seeking sections in the amino acid sequence, wherein amino acid residues can be replaced (mutation) without adversely affecting the functionality of the protein, based on multiple sequence alignment and/or structural information. According to the presently disclosed invention, mutations are introduced into the protein sequence to insert split sites and/or ligation sites into the protein sequence, as well as reducing the hydrophobicity of the ligation-conducive polypeptides, and to reduce the cost of preparation of D-amino acids proteins, by reducing the number of Ile residues in the protein. Uses of the D-amino acids proteins are also provided, such as, without limitation bio-orthogonal molecular data storage, SELEX for aptamer development and crystal growth strategy in X-ray protein crystallography.

Thus, according to an aspect of some embodiments of the present invention there is provided a method of chemically producing a protein, which is effected by ligating at least two ligation-conducive segments of the protein, wherein each of the ligation-conducive segments is chemically-synthesizable, and obtainable by:
i. identifying at least one ligation-conducive sequence in the amino-acid sequence of the protein, parsing the amino-acid sequence of the protein at the ligation-conducive sequence to thereby obtain a plurality of ligation-conducive segments; and
ii. if each of the ligation-conducive segments is chemically-synthesizable, chemically synthesizing each of the ligation-conducive segments;
iii. if any one of the ligation-conducive segments is not chemically-synthesizable, identifying at least one structurally-lose section in the ligation-conducive segment, substituting at least one amino acid in the structurally-lose section with a ligation-conducive amino acid residue so as to introduce a ligation-conducive sequence in the structurally-lose section, parsing the amino-acid sequence of the protein at the ligation-conducive sequence; and chemically synthesizing each of the ligation-conducive segments.

In some embodiments of the present invention, in Step (i), at least one of the ligation-conducive sequences is in a structurally-lose section in the protein.

In some embodiments of the present invention, the method provided herein includes Step (iii).

In some embodiments of the present invention, the method provided herein further includes, prior to Step (i),
a) splitting the amino-acid sequence of the protein into at least two domain-forming segments;
b) if each of the domain-forming segments is chemically-synthesizable, chemically synthesizing each of the domain-forming segments; and
c) co-folding the domain-forming segments to thereby obtain the protein.

In some embodiments of the present invention, the method provided herein includes Step (a), of splitting the amino-acid sequence of the protein into at least two domain-forming segments.

According to some embodiments of the present invention, if one of the domain-forming segments is not chemically-synthesizable, the method is further effected by:
d) identifying at least one ligation-conducive sequence in the domain-forming segment, and parsing the amino-acid sequence of the domain-forming segment at the ligation-conducive sequence to thereby obtain a plurality of chemically-synthesizable ligation-conducive segments;
e) if the domain-forming segment is essentially devoid of a ligation-conducive sequence, or any one of the ligation-conducive segments is not chemically-synthesizable, identifying at least one structurally-lose section in the domain-forming segment or the ligation-conducive segment;
f) substituting at least one amino acid in the structurally-lose section or the ligation-conducive segment with a ligation-conducive amino acid residue so as to introduce a ligation-conducive sequence in the structurally-lose section or the ligation-conducive segment, and parsing the amino-acid sequence of the domain-forming segment at the ligation-conducive sequence to thereby obtain a plurality of sequences of chemically-synthesizable ligation-conducive segments; and
g) chemically synthesizing each of the chemically-synthesizable ligation-conducive segments.

In some embodiments of the present invention, the method provided herein includes Step (f).

According to some embodiments of the present invention, the synthetic protein exhibits at least 1%, 5%, or at least 10% of the activity of the corresponding biologically produced protein.

According to some embodiments of the present invention, the activity is selected from the group consisting of a catalytic activity, a specific binding activity, and a structural activity.

According to some embodiments of the present invention, the protein includes at least 240 amino-acid residues.

According to some embodiments of the present invention, the protein includes at least about 400 amino-acid residues.

According to some embodiments of the present invention, the method provided herein further includes, in at least one of the ligation-conducive segments, substituting at least one hydrophobic amino-acid residue with a less hydrophobic amino acid, according to the following order of hydrophobicity: Ile>Leu>Phe>Val>Met>Pro>Trp>His(0)>Thr>Glu(0)>Gln>Cys>Tyr>Ala>Ser>Asn>Asp(0)>Arg+>Gly>His+>Glu>Lys+>Asp-.

According to some embodiments of the present invention, the synthetic protein is produced using at least 90% non-Gly D-amino-acid residues.

According to some embodiments of the present invention, the protein has essentially a mirror-imaged 3D structure compared to a 3D structure of a corresponding biologically produced protein.

According to some embodiments of the present invention, the method provided herein further includes substituting at least one Ile residue with a D-amino-acid residue selected from the group consisting of a D-Ala residue, a D-Val residue, a D-Leu residue, a D-Thr residue, a D-Phe residue, a D-Met residue, a Gly residue, and a D-Pro residue.

According to another aspect of some embodiments of the present invention, there is provided a protein, prepared according to the method provided herein, wherein the protein is at least about 240 amino-acid residues long.

According to some embodiments of the present invention, the chemically synthesized protein provided herein includes at least two domain-forming segments being non-covalently attached polypeptide chains, wherein the domain-forming segments being covalently attached polypeptide chains in at least one corresponding biologically produced protein.

According to some embodiments of the present invention, the protein provided herein is selected from the group consisting of an enzyme, a transport protein, a structure/mechanics protein, a hormone, a signaling protein, an antibody, a fluid-balancing protein, a pH-balancing protein, a cellular channel and a cellular pump.

According to some embodiments of the present invention, the protein is an enzyme that is capable of catalyzing a reaction catalyzed by a corresponding biologically produced enzyme.

According to some embodiments of the present invention, the chemically synthesized enzyme is an RNA polymerase, capable of synthesizing RNA from ribonucleotides using a DNA template.

According to some embodiments of the present invention, the chemically synthesized RNA polymerase is a T7 RNA polymerase, or a Pfu DNA polymerase mutant.

According to some embodiments of the present invention, the chemically synthesized Pfu DNA polymerase mutant is having at least one mutation selection from the group consisting of V93Q, E102A, D141A, E143A, Y410G, A486L and E665K.

In some embodiments, the Pfu DNA polymerase further includes at least one mutation selected from the group consisting of D215A, A486Y and L490W (SEQ ID No. 77).

In some embodiments, the Pfu DNA polymerase further includes a DNA binding structural domain, wherein the DNA binding structural domain is sso7d structural domain (SEQ ID No. 78).

According to some embodiments of the present invention, the chemically synthesized enzyme is a DNA polymerase, capable of synthesizing DNA from deoxyribonucleotides.

According to some embodiments of the present invention, the chemically synthesized DNA polymerase is a Pfu DNA polymerase.

According to another aspect of embodiments of the present invention, there is provided a method of chemically producing a D-amino acids protein (a mirror image protein), which includes ligating at least two ligation-conducive segments of the D-amino acids protein, wherein each of the ligation-conducive segments includes at least 90% non-Gly D-amino-acid residues and is chemically-synthesizable, and is obtainable by:
i. identifying at least one ligation-conducive sequence in the amino-acid sequence of a corresponding L-amino-acid protein, parsing the amino-acid sequence at the ligation-conducive sequence to thereby obtain a plurality of ligation-conducive segments; and;
ii. if each of the ligation-conducive segments is chemically-synthesizable, chemically synthesizing each of the ligation-conducive segments using at least 90% non-Gly D-amino-acid residues;
iii. if any one of the ligation-conducive segments is not chemically-synthesizable, identifying at least one structurally-lose section in the ligation-conducive segment, substituting at least one amino acid in the structurally-lose section with a ligation-conducive amino acid residue so as to introduce a ligation-conducive sequence in the structurally-lose section, parsing the amino-acid sequence of the ligation-conducive segment at the ligation-conducive sequence; and chemically synthesizing each of the ligation-conducive segments using at least 90% non-Gly D-amino-acid residues.

According to some embodiments of the present invention, the method for producing a mirror image protein includes, in Step (i), that at least one of the ligation-conducive sequences is in a structurally-lose section in the corresponding L-amino-acid protein.

According to some embodiments of the present invention, the method for producing a mirror image protein includes Step (iii).

According to some embodiments of the present invention, the method for producing a mirror image protein further includes, prior to Step (i),
a) splitting the amino-acid sequence of the L-amino-acid protein into at least two domain-forming segments;
b) if each of the domain-forming segments is chemically-synthesizable, chemically synthesizing each of the domain-forming segments using at least 90% non-Gly D-amino-acid residues; and
c) co-folding the domain-forming segments, thereby obtaining the D-amino acids protein.

According to some embodiments of the present invention, in the method for producing a mirror image protein, if one of the domain-forming segments is not chemically-synthesizable;
d) identifying at least one ligation-conducive sequence in the domain-forming segment, and parsing the amino-acid sequence of the domain-forming segment at the ligation-conducive sequence to thereby obtain a plurality of chemically-synthesizable ligation-conducive segments;
e) if the domain-forming segment is essentially devoid of a ligation-conducive sequence, or any one of the ligation-conducive segments is not chemically-synthesizable, identifying at least one structurally-lose section in the domain-forming segment or the ligation-conducive segment;
f) substituting at least one amino acid in the structurally-lose section or the ligation-conducive segment with a ligation-conducive amino acid residue so as to introduce a ligation-conducive sequence in the structurally-lose section or the ligation-conducive segment, and parsing the amino-acid sequence of the domain-forming segment at the ligation-conducive sequence; and
g) chemically synthesizing each of the ligation-conducive segments using at least 90% non-Gly D-amino-acid residues thereby obtaining the domain-forming segment.

According to some embodiments of the present invention, the method for producing a mirror image protein includes Step (iii).

According to some embodiments of the present invention, in the method for producing a mirror image protein, the D-amino acids protein exhibits at least 1%, at least 5% or at least 10% of the activity of the corresponding L-amino acids protein.

According to some embodiments of the present invention, the activity of the mirror image protein is selected from the group consisting of a catalytic activity, a specific binding activity, and a structural activity.

According to some embodiments of the present invention, the D-amino acids protein provided herein includes at least 240, 300, 400 or at least 500 amino-acid residues.

According to some embodiments of the present invention, the method for producing a mirror image protein further includes, substituting in at least one of the ligation-conducive segments, at least one hydrophobic D-amino-acid residue with a less hydrophobic amino acid, according to the following order of hydrophobicity: D-Ile>D-Leu>D-Phe>D-Val>D-Met>D-Pro>D-Trp>D-His(0)>D-Thr>D-Glu(0)>D-Gln>D-Cys>D-Tyr>D-Ala>D-Ser>D-Asn>D-Asp(0)>D-Arg+>Gly>D-His+>D-Glu>D-Lys+>D-Asp-.

According to some embodiments of the present invention, the D-amino acids protein exhibits essentially a mirror-imaged 3D structure compared to a 3D structure of the corresponding L-amino acids protein.

According to some embodiments of the present invention, the method for producing a mirror image protein further includes substituting at least one Ile residue with a D-amino-acid residue selected from the group consisting of a D-Ala residue, a D-Val residue, a D-Leu residue, a D-Thr residue, a Gly residue, a D-Phe residue, a D-Met residue, and a D-Pro residue.

According to another aspect of some embodiments of the present invention, there is provided a D-amino acids protein, prepared according to the method provided herein.

In some embodiments of the present invention, the D-amino acids protein is having essentially a mirror-imaged 3D structure compared to a 3D structure of a corresponding L-amino acids protein (e.g., a corresponding biologically-produced protein).

According to some embodiments of the present invention, the D-amino acids protein includes at least two domain-forming segments being non-covalently attached polypeptide chains, wherein the domain-forming segments being covalently attached polypeptide chains in at least one corresponding L-amino acids protein.

According to some embodiments of the present invention, the D-amino acids protein is selected from the group consisting of an enzyme, a transport protein, a structure/mechanics protein, a hormone, a signaling protein, an antibody, a fluid-balancing protein, a pH-balancing protein, a cellular channel and a cellular pump.

According to some embodiments of the present invention, the D-amino acids protein is a D-amino acids enzyme that is capable of catalyzing an enantiomeric reaction compared to a corresponding L-amino acids enzyme, namely catalyzing a reaction comparable to the enzymatic reaction of the corresponding biologically produced enzyme, using an enantiomorph of the corresponding substrate, to form an enantiomorph of the corresponding product.

According to some embodiments of the present invention, the D-amino acids enzyme is a D-amino acids RNA polymerase, capable of synthesizing L-RNA from L-ribonucleotides using an L-DNA template.

According to some embodiments of the present invention, the D-amino acids RNA polymerase is a D-amino acids T7 RNA polymerase, or a D-amino acids Pfu DNA polymerase mutant.

According to some embodiments of the present invention, the D-amino acids Pfu DNA polymerase mutant having at least one mutation selection from the group consisting of V93Q, E102A, D141A, E143A, Y410G, A486L and E665K.

According to some embodiments of the present invention, the D-amino acids protein is a T7 RNA polymerase that includes at least one split site, a first split site between K363 and P364 and a second split site between N601 and T602.

According to some embodiments of the present invention, the D-amino acids enzyme is a D-amino acids DNA polymerase, capable of synthesizing L-DNA from L-deoxyribonucleotides.

According to some embodiments of the present invention, the D-amino acids DNA polymerase is a D-amino acids Pfu DNA polymerase.

According to another aspect of some embodiments of the present invention, there is provided a T7 RNA polymerase, which includes at least two polypeptide chains formed by a split between K363 and P364 and/or a split between N601 and T602.

In some embodiments, the T7 RNA polymerase provided herein further includes at least one mutation selected from the group consisting of I6V, I14L, I74V, I82V, I109V, I117L, I141V, I210M, I244L, I281V, I320V, I322L, I330V and I367L.

According to another aspect of embodiments of the present invention, there is provided a T7 RNA polymerase, having an amino-acid sequence characterized by at least 80% or at least 90% sequence identity compared to SEQ ID No. 83.

According to another aspect of some embodiments of the present invention, there is provided a Pfu DNA polymerase, which includes at least two polypeptide chains formed by a split between K467 and M468. The two polypeptide chains are not connected to one another via a covalent bond between their main-chain.

In some embodiments, the Pfu DNA polymerase further includes at least one mutation selected from the group consisting of E102A, E276A, K317G, V367L and I540A.

In some embodiments, the Pfu DNA polymerase provided herein further includes at least one mutation selected from the group consisting of I38F, I62V, I65V, 180V, I127V, I137M, I158L, I171A, I176V, I191V, I197V, I198V, I205V, I206V, I228V, I232L, I244M, I256V, I264A, I268L, I282V, I331A, I401V, I434V, I446F, I478K, I557V, I598V, 1605T, I611V, I619A, I631L, I643V, I648T, I656V, I677T, I716Y, I734V, I745V and I772P.

In some embodiments, the Pfu DNA polymerase further includes at least one mutation selected from the group consisting of V93Q, D141A, E143A, Y410G, A486L and E665K.

In some embodiments, the Pfu DNA polymerase exhibits RNA polymerization activity.

In some embodiments, the Pfu DNA polymerase further includes mutations selected from the group consisting of D215A, A486Y and/or L490W.

In some embodiments, the Pfu DNA polymerase exhibits deficient 3′ to 5′ exonuclease activity and increased dideoxynucleoside triphosphates (ddNTPs) selectivity.

In some embodiments, the Pfu DNA polymerase further comprising a DNA binding structural domain, wherein the DNA binding structural domain is sso7d structural domain (SEQ ID No. 78).

In some embodiments, the Pfu DNA polymerase modified with an sso7d structural domain exhibits improved PCR amplification activities.

According to another aspect of some embodiments of the present invention, there is provided a Pfu DNA polymerase, having an amino-acid sequence characterized by at least 80% or at least 90% sequence identity compared to SEQ ID No. 51, or having an amino-acid sequence characterized by at least 80% or at least 90% sequence identity compared to SEQ ID No. 79.

According to another aspect of some embodiments of the present invention, there is provided a use of the D-amino acids protein provided herein, wherein the D-amino acids protein is an enzyme, and the use is in catalyzing a synthesis of a product being an enantiomorph of a molecule being synthesized by a corresponding L-amino acids enzyme, or in catalyzing a reaction of a substrate being an enantiomorph of a corresponding substrate of a corresponding L-amino acids enzyme.

According to another aspect of some embodiments of the present invention, there is provided a process of producing an L-polydeoxyribonucleic acid molecule enzymatically, effected by:
providing a D-amino acids DNA polymerase prepared according to the method provided herein, and capable of synthesizing L-DNA from L-deoxyribonucleotides; and reacting the D-amino acids DNA polymerase with a template L-DNA molecule, L-DNA primers and a plurality of L-deoxyribonucleotides, to thereby enzymatically producing the L-DNA molecule.

In some embodiments of the process aspect, the D-amino acids DNA polymerase is a Pfu DNA polymerase.

In some embodiments of the process aspect, the Pfu DNA polymerase is essentially as provided herein.

According to another aspect of some embodiments of the present invention, there is provided a process of producing an L-polyribonucleic acid (L-RNA) molecule enzymatically, which is effected by:
providing a D-amino acids RNA polymerase prepared according to the method provided herein, and capable of synthesizing L-RNA from L-ribonucleotides; and reacting the D-amino acids RNA polymerase with a template L-DNA molecule. L-DNA/RNA primers and a plurality of L-ribonucleotides, to thereby enzymatically producing the L-RNA molecule.

In some embodiments of the process aspect, the D-amino acids RNA polymerase is a T7 RNA polymerase, or a Pfu DNA polymerase mutant, the Pfu DNA polymerase mutant is having at least one mutation selected from the group consisting of V93Q, E102A, D141A, E143A, Y410G, A486L and E665K.

In some embodiments of the process aspect, the T7 RNA polymerase is essentially as provided herein.

According to another aspect of some embodiments of the present invention, there is provided a method for forming a racemic crystal of a molecule of interest, which is effected by co-crystallizing the molecule of interest and an enantiomorph of the molecule of interest, thereby forming the racemic crystal of an enantiomeric pair, wherein the enantiomorph of the molecule of interest is a D-amino-acids protein provided according to the methods presented herein, or a product of such D-amino-acids protein.

According to another aspect of some embodiments of the present invention, there is provided a molecular probe that includes the D-amino acids protein as provided herein, having attached thereto a labeling moiety and having an affinity to an analyte being an enantiomorph of a corresponding analyte of a corresponding L-amino acids protein.

According to another aspect of some embodiments of the present invention, there is provided a method for producing an L-nucleic acid aptamer or a D-peptide binding moiety, which is effected by:
providing a D-amino acids protein, prepared according to the method presented herein; and
subjecting the D-amino acids protein to a systematic evolution of ligands by exponential enrichment process,
thereby obtaining the L-nucleic acid aptamer or a D-peptide binding moiety.

According to another aspect of some embodiments of the present invention, there is provided a method of amplification of a DNA sequence or an RNA sequence, that includes reacting a template of the DNA or RNA sequence with a DNA or RNA polymerase prepared according to the herein-provided method, wherein the reaction is effected essentially without a natural enzyme and/or a natural DNA/RNA contamination.

According to another aspect of some embodiments of the present invention, there is provided a method of sequencing L-DNA or L-RNA, using a D-amino acid DNA or a D-amino acid RNA polymerase, as provided herein, phosphorothioate L-dNTPs, or phosphorothioate L-NTPs, and 5′-labelled two primers with two different dyes.

According to another aspect of some embodiments of the present invention, there is provided a method of sequencing L-DNA, using a D-amino acid DNA polymerase, as provided herein, L-dideoxynucleoside triphosphates, and 5′-labelled two primers with two different dyes.

In some embodiments, the dyes are FAM and Cy5.

According to another aspect of some embodiments of the present invention, there is provided a data storage system, which includes:
at least one L-nucleic acid (for example, L-DNA, L-RNA and any chimeras thereof with D-nucleic acid segments) molecule having a sequence encoding information data;
a D-amino acid RNA polymerase and/or a D-amino acid DNA polymerase for synthesizing and/or sequencing the L-nucleic acids, wherein the D-amino acid RNA polymerase and/or the D-amino acid DNA polymerase is produced according to the method provided herein.

In some embodiments of the system, the L-nucleic acid molecule is prepared chemically, or by mirror-image enzyme-catalyzed reactions. In some embodiments of the L-DNA data storage system, the information-storing L-DNA segments are prepared by mirror-image assembly PCR using D-enzymes.

In some embodiments of the system, the L-nucleic acid molecule is sequenced chemically, or by sequencing-by-synthesis methods using mirror-image enzymes.

In some embodiments of the system, the D-amino acid RNA polymerase is the T7 RNA polymerase provided herein.

In some embodiments of the system, the D-amino acid DNA polymerase is the Pfu DNA polymerase provided herein.

According to another aspect of some embodiments of the present invention, there is provided a chiral steganography approach, which is effected by:
at least one D-nucleic acid molecule having a sequence encoding cover information data;
at least one L-nucleic acid molecule and/or a D-/L-chimeric nucleic acid molecule having a sequence encoding a cipher key to decrypt the stego information data.
a D-amino acid RNA polymerase and/or a D-amino acid DNA polymerase for synthesizing and/or sequencing the L-DNA molecule, wherein the D-amino acid RNA polymerase and/or the D-amino acid DNA polymerase is produced as provided herein.

In some embodiments, the L-nucleic acid molecule is prepared chemically, or by mirror-image enzyme-catalyzed reactions.

In some embodiments, the L-nucleic acid molecule is sequenced chemically, or by sequencing-by-synthesis methods using mirror-image enzymes.

In some embodiments, the D-/L-chimeric nucleic acid molecule is prepared chemically, or by natural/mirror-image enzyme-catalyzed reactions.

In some embodiments, the L-DNA/RNA part of D-/L-chimeric nucleic acid molecule is sequenced chemically, or by sequencing-by-synthesis methods using mirror-image enzymes.

In some embodiments, the D-amino acid RNA polymerase is the T7 RNA polymerase as provided herein.

In some embodiments, the D-amino acid DNA polymerase is the Pfu DNA polymerase as provided herein.

In some embodiments, the system is potential to be combined with DNA cryptography to provide an extra layer of security using encrypted data.

According to another aspect of some embodiments of the present invention, there is provided a method for studying L-RNA hydrolysis, which is effected by:
at least one L-RNA molecule having a higher-ordered structure and long-length sequence;
a D-amino acid RNA polymerase and/or a D-amino acid DNA polymerase for synthesizing the L-RNA molecule, wherein the D-amino acid RNA polymerase and/or the D-amino acid DNA polymerase is produced according to the method provided herein.

According to another aspect of some embodiments of the present invention, there is provided a method for studying RNA degradation, effected by:
at least one L-RNA molecule having a higher-ordered structure and long-length sequence;
a D-amino acid RNA polymerase and/or a D-amino acid DNA polymerase for synthesizing the L-RNA molecule, wherein the D-amino acid RNA polymerase and/or the D-amino acid DNA polymerase is produced according to the method provided herein.

In some embodiments, the method can be used to evaluate the effectiveness of RNase-inhibiting reagents.

According to another aspect of some embodiments of the present invention, there is provided a transcriptional AND-logic, effected by:
a D-amino acid RNA polymerase, wherein the D-amino acid RNA polymerase a is produced according to the method provided herein.

In some embodiments, the D-amino acid RNA polymerase is the T7 RNA polymerase provided herein.

In some embodiments, the D-amino acid RNA polymerase comprising at least one split site, a first split site between K363 and P364 and a second split site between N601 and T602.

In some embodiments, the D-amino acid RNA polymerase comprising at least one split site, the above-mentioned sites in the same loop, namely from position 357 to position 366 and/or from position 564 to position 607.

According to another aspect of some embodiments of the present invention, there is provided a method of producing L-RNA marker/ladder, comprising:
providing a D-amino acids RNA polymerase prepared according to the method provided herein, and capable of synthesizing L-RNA from L-ribonucleotides; and
reacting the D-amino acids RNA polymerase with each template L-DNA molecule of different lengths, L-DNA/RNA primers and a plurality of L-ribonucleotides;
to thereby enzymatically produce the L-RNA molecules of different lengths, respectively, and mix them together in a certain concentration after purification.

In some embodiments, the D-amino acids RNA polymerase is a T7 RNA polymerase essentially as provided herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying figures. With specific reference now to the figures in detail, it is stressed that the particulars shown are by way of example and for purposes of illustrative discussion of embodiments of the invention. In this regard, the description taken with the figures makes apparent to those skilled in the art how embodiments of the invention may be practiced.

In the figures:

FIG. 1 is a flowchart illustrating the method provided herein, according to some embodiments of the present invention;

FIGS. 2A-B present the design flow of the synthetic route of the mutant Pfu-N fragment (FIG. 2A), wherein additional NCL sites were introduced (E102A, E276A, K317G, V367L) to form ligation-conducive segments, and 25 isoleucine residues were substituted, and the design flow of the synthetic route of the mutant Pfu-C fragment (FIG. 2B), wherein an additional NCL site (I540A) was introduced, as well as the mutation of other 15 isoleucine residues, whereas these mutations were introduced to facilitate protein synthesis in SPPS and ligation process and reduce synthesis cost of the mirror-image version;

FIGS. 3A-C present the design flow of the synthetic route of the 369-aa (including a His6 tag added to the N terminus) mutant T7-split-N fragment (FIG. 3A), the 238-aa mutant T7-split-M fragment (FIG. 3B), and the 282-aa mutant T7-split-C fragment (FIG. 3C), including replacement of isoleucine residues, new NCL and a new split site between K363 and P364, which were introduced to facilitate protein synthesis in SPPS and ligation process, and reduce synthesis cost of the mirror-image version;

FIG. 4 is a flowchart illustrating molecular data storage, according to some embodiments of the present invention, using L-DNA as an exemplary type of XNA; and

FIG. 5 presents a flowchart illustrating DNA based steganography, according to some embodiments of the present invention, embedding a chimeric D-DNA/L-DNA key molecule in a seemingly ordinary D-DNA storage library to convey a secret message.

### DESCRIPTION OF SPECIFIC EMBODIMENTS OF THE INVENTION

The present invention, in some embodiments thereof, relates to biochemistry and more particularly, but not exclusively, to methods of total chemical synthesis of large proteins and their mirror-image counterparts, and uses thereof.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Alpha-amino acids—the basic building blocks of proteins—are chiral molecules that exist in two forms: L-enantiomer (‘L’ for levorotatory or left-handed) and D-enantiomer (‘D’ for dextrorotatory or right-handed). The two non-superimposable forms of amino acid differing in handedness or chirality are mirror images of one another and have otherwise identical physical and chemical properties. Life on earth, however, uses only L-amino acids and the achiral amino acid glycine to construct proteins that perform a great variety of biological functions. Although present in nature, notably in the peptidoglycans of cell walls and in peptide antibiotics of bacterial origin, in proteins of lower animals such as insects, snails and amphibians, and even in the brain as neurotransmitters, D-amino acids in various organisms are thought to be converted from parent L-enantiomers through enzyme catalyzed post-translational reactions. The fascinating question of why and how life on Earth favors these left-handed molecules has been a subject of intense debate for decades among chemists, physicists, biologists, and even astronomers. While the origin of homochirality of alpha-amino acids continually remains a mystery, scientists have learned a great deal already from studying the physicochemical and biological properties of unnatural or artificial D-peptides and D-proteins that contain only chiral D-amino acids.

While reducing the present invention to practice, the inventors reasoned that in order to build mirror-image biology systems in the laboratory, a core step is to establish a chirally-inverted version of the central dogma of molecular biology (5-7), taking advantage of the chemical syntheses of mirror-image nucleic acids and proteins as two technical pillars (5). The present inventors have reasoned that one way to overcome the bottleneck of synthesizing long L-nucleic acid molecules is through enzymatic polymerization by mirror-image polymerases, which lead to the conceivement of the present invention, and to the realization of a proof-of-concept. Nonetheless, the earlier versions of mirror-image polymerase systems were chosen as models for total chemical synthesis as a reluctant compromise between polymerase activity and size (5). The intrinsic poor processivity and fidelity of small polymerases such as ASFV pol X and Dpo4 (with error rates on the order of 10⁻⁴ to 10⁻²) have made them unsuitable for the faithful assembly, amplification, and transcription of long mirror-image genes (5, 17, 18, 21).

Thus, the present inventors have contemplated a method that would render the total chemical synthesis of seemingly any protein possible, and the route to D-amino acids proteins has been opened thereby.

The method of total chemical synthesis of large proteins, according to embodiments of the present invention, is a systematic elimination of hitherto insurmountable obstacles in the field, and is based on introducing specific mutations in the amino acid sequence of the target protein, such that the length problems are mitigated without nullifying the specific activity of the protein.

### Split Protein Design:

The present inventors have reasoned that taking advantage of split protein designs may drastically simplify the problem of chemically synthesizing large proteins into the synthesis of two or smaller protein fragments, which can co-fold in vitro into a functionally intact enzyme. Moreover, the split-protein strategy will allow the synthesis, purification, ligation, and desulfurization of each split-protein fragment to be performed in parallel, greatly reducing the overall time needed for synthesizing large proteins, as well as the cost and time for corrections when failure on certain fragment(s) occurs. Some enzymes have natural or engineered split versions, including the Pfu DNA polymerase; for example, a known split site between K467 and M468 in the coiled coil motif of its fingers domain divides the polymerase into two fragments (a 467-aa Pfu-N fragment and a 308-aa Pfu-C fragment, without significantly altering its PCR activity and fidelity. The above-mentioned split site may also be selected near the above-mentioned sequence positions in the coiled coil motif of the fingers domain of the Pfu DNA polymerase, for example, between position 449 and position 498.

Thus, according to some embodiments of the present invention, the method of chemically producing a protein, includes splitting the amino-acid sequence of the protein into at least two domain-forming segments, each of which is short enough to be synthesized chemically from ligation of smaller polypeptide segments, and yet long enough to fold into a functional domain in a functional protein, when the domain-forming segments are co-filed together under folding-conducive conditions.

According to some embodiments of the present invention, if the domain-forming segment is chemically-synthesizable by SPPS or AFPS, or about 120, 150 or 200 amino acid residues long or less, which typically means it can be chemically synthesized, and be suitable for co-folding with other domain-forming segments to thereby obtain the protein.

The term “chemically-synthesizable”, as used herein, refers primarily to the length of a polypeptide that can be achieved by any non-biologic synthesis process, such as solid phase peptide synthesis (SPPS), or automated fast-flow peptide synthesis (AFPS). In general, it is known that a polypeptide of about 10-120 amino acid residues long can be produced by solid phase peptide synthesis (SPPS), and a polypeptide of about 10-180 amino acid residues long, can be afforded by automated fast-flow peptide synthesis (AFPS). In some embodiments, the term “chemically-synthesizable” refers to a polypeptide chain of about 120, 150 or 200 amino acid long. In some embodiments, the term “chemically-synthesizable” also refers to the ability to purify, and optionally isolate the chemically synthesized polypeptide.

If the domain-forming segment is longer than is suitable for chemical synthesis, it is further segmented into ligation-conducive segments, which are ligated to form the (relatively longer) domain-forming segment.

In the context of embodiments of the present invention, the term “fragment” is used herein and throughout interchangeably with the term “domain-forming segment”. The term “domain-forming segment”, as used herein, refers to a continuous polypeptide chain which folds into a recognizable protein domain(s), as this term is known in the art. According to some embodiments, a domain-forming segment can fold in vitro into one or more domains that resemble or essentially identical to the structure of these domains when the polypeptide folds in vivo, or under biological/physiological conditions.

In the context of embodiments of the present invention, a domain-forming segment can be a multidomain protein or comprise a single recognizable domain. The recognition or identification of domains is within the capacity of a person of ordinary skills in the art, and is typically done using one or more publically accessible bioinformatics tools, such as multiple sequence alignments, SCOP [scop(dot)berkeley(dot)edu/], CATH [www(dot)cathdb(dot)info], ExPASy [www(dot)expasy(dot)org], BLAST [blast(dot)ncbi(dot)nlm(dot)nih(dot)gov], PFAM [pfam(dot)xfam(dot)org], PDB [www(dot)rcsb(dot)org], and the likes, all of which are within the reach and discernment of the skilled artisan.

As discussed hereinabove, some proteins are naturally built from more than one polypeptide chain, which are equivalent to the multidomain- or domain-forming segments discussed herein. Such natural or intended splitting into domain-forming segments can be exploited in the method presented herein.

Some proteins may be built from one continuous polypeptide chain, however, their evolutionary family members may include some that have evolved to be built from more than one polypeptide chain. Information regarding possible splitting may stem from multiple sequence alignment of family members, as well as from intentional splitting of family members of the protein of interest for chemical production. Another source of information regarding optional splitting sites may come from structural information of the protein of interest or family members of the protein, aided by structural alignment—revealing that certain sections in the protein are less preserved and therefore expected not to disrupt the activity of the protein if a split site is introduced intentionally into the sequence.

Sections in the protein that can serve as possible split sites, are referred to herein as structurally-lose sections, regardless if the information that lead to their identification comes from sequence data and/or structural data. Thus, a “structurally-lose section” is identifiable by using multiple sequence alignment and/or from structural information of the protein of interest and/or from members of the protein's family.

According to some embodiments of the present invention, if a protein is too long to practically be chemically produced directly by SPPS or by the combination of SPPS and ligation, a split site can be introduced into the sequence of the protein of interest, with the expectation that the domain-forming segments, once chemically synthesized, would co-fold into the protein.

### Chemical Ligation:

As was found while reducing the present invention to practice, even when a protein can be realized by co-folding, after implementing the split design approach, each or one of the domain-forming segments may be too long to realize by chemical synthesis.

Native chemical ligation (NCL) is an extension of the chemical ligation field, a concept for constructing a large polypeptide formed by the assembling of two or more unprotected peptides segments. Especially, NCL is a powerful ligation method for synthesizing native backbone proteins or modified proteins of small and moderate size. In native chemical ligation, the thiol group of an N-terminal cysteine residue of an unprotected peptide attacks the C-terminal thioester of a second unprotected peptide. This reversible transthioesterification step is chemoselective and regioselective and leads to form a thioester intermediate. This intermediate rearranges by an intramolecular S,N-acyl shift that results in the formation of a native amide (peptide) bond at the ligation site.

In the context of embodiments of the present invention, the term “ligation-conducive sequence” refers to a location in the protein sequence that exhibit an amino acid sequence which can be formed by NCL. For example, am N-terminal cysteine residue can be used to effect chemical ligation under known conditions. The identification and exploitation of ligation-conducive sequences is well within the reach of any person of ordinary skills in the art, and additional information is readily available in the literature (e.g., the review article “Native Chemical Ligation and Extended Methods: Mechanisms, Catalysis, Scope, and Limitations” by Agouridas, V. et al. [Chem Rev. 2019,119(12), pp. 7328-7443]).

Thus, according to some embodiments of the present invention, the protein, or long domain-forming segments thereof, can be synthesized by first identifying ligation-conducive sequences in the amino-acid sequence of the protein, and then parsing the sequence at these ligation-conducive sequence, or at least some thereof, to thereby obtain a plurality of sequences of ligation-conducive segments of the protein, each of which is short enough to be effectively chemically synthesized and purified. Each of the ligation-conducive segments that can be chemically synthesized, are thereafter ligated to form the protein or a domain-forming segment.

In general, according to some embodiments of the present invention, a ligation-conducive sequence/segment is chemically-synthesizable, or about 10-120, about 10-150 or about 10-200 amino acids long.

If the protein does not exhibit a ligation-conducive sequence at desirable positions, based on the length of the segments, ligation-conducive sequences can be introduced by mutation of the amino acid sequence of the protein. Thus, according to some embodiments of the present invention, if any one of the ligation-conducive segments is not chemically-synthesizable, namely longer than about 120, 150 or 200 amino acid residues long, or of other length that cannot be effectively synthesized and purified, the method is effected by identifying at least one structurally-lose section in the ligation-conducive sequence, substituting at least one amino acid in said structurally-lose section with a ligation-conducive amino acid residue so as to introduce a ligation-conducive sequence in said structurally-lose section, followed by parsing the amino-acid sequence of the protein at the ligation-conducive sequence afforded by mutation, further followed by chemically synthesizing each of said ligation-conducive segments.

For example, the synthesis of the Pfu-N fragment with 467 aa (54 kDa) alone, which is much larger than Dpo4 with 352 aa (40 kDa), still poses considerable challenges. One of the challenges is that NCL of synthetic peptides prepared by SPPS requires an N-terminal cysteine residue at the ligation site, and yet the wild-type (WT) Pfu DNA polymerase only has four cysteine residues (C429 and C443 in the Pfu-N fragment (SEQ ID No. 57); C507 and C510 in the Pfu-C fragment (SEQ ID No. 67)). Although the inventors took advantage of a previously reported metal-free radical-based desulfurization approach to convert unprotected cysteine to alanine residue after NCL so that another eight ligation sites with alanine residues (A40, A163, A223, and A408 in the Pfu-N fragment; A501, A596, A652 and A715 in the Pfu-C fragment) could be also used, some of the peptide segments were still too long to be prepared by SPPS. Therefore, the inventors designed a mutant version of the Pfu DNA polymerase with five point mutations (E102A, E276A, K317G, and V367L in the Pfu-N fragment; I540A in the Pfu-C fragment) based on sequence alignment to introduce additional ligation sites, or ligation-conducive sequences, without significantly altering the PCR activity of the polymerase (split Pfu-5m; SEQ ID No. 48).

### Hydrophobicity and Bulk:

Another challenge is the synthesis and ligation of hydrophobic peptide segments under aqueous conditions. Current methods to overcome this problem mainly focus on introducing various mutations and/or chemical modifications to the target peptide in order to reduce the number of highly hydrophobic and/or bulky amino acid residues. According to some embodiments of the present invention, chemical modifications are effected by, for example, Hmb-N^{α}-protection, removable solubilizing tags, pseudoprolines, and depsipeptide (O-acyl isopeptide), although their practical use is often constrained by the laborious procedures, low yield, and requirement of expensive amino acid derivatives.

According to some embodiments of the present invention, in order to facilitate the chemical synthesis, ligation and co-folding of various segments of the chemically produced protein, some highly hydrophobic and/or bulky residues are replaced (mutated) with less hydrophobic and/or less bulky residues, wherein the criteria for such substitutions may rely on MSA, structural information and other mutation data.

Hydrophobicity and bulkiness, while related to one another, and in most cases go hand-in-hand, are not necessarily the same property, as these properties may vary differently under difference environments, depending on the pH, ionic strength, counter ions, water activity, temperature, and other factors. Different references in the literature gives slightly different values and ranking of hydrophobicity and bulkiness of amino acid residues in the context of a polypeptide chain, although the general notion that isoleucine is “one of the most bulky and hydrophobic amino acids” holds true by all. Exemplary sources of information relating to hydrophobicity and bulkiness include, without limitation, Kyte, J. and Doolittle, R. F., “A simple method for displaying the hydropathic character of a protein” [J. Mol. Biol., 1982, 157(1), pp. 105-132] and Ellington, A. and Cherry, J. M., “Characteristics of amino acids” [Curr Protoc Mol Biol, 2001, A.1C.1-A.1C.12]. For instance, embodiments of the present invention may base criteria for mutating amino acids for reducing bulkiness according to the following, non-limiting exemplary order: I>L>C>T>V>P>S>A>G, and for reducing hydrophobicity according to the following, non-limiting exemplary order: I>V>L>F>C>M>A>G>T.

In general, as known in the art, the residues replacement guideline go according to the following order of hydrophobicity: Ile>Leu>Phe>Val>Met>Pro>Trp>His(0)>Thr>Glu(0)>Gln>Cys>Tyr>Ala>Ser>Asn>Asp(0)>Arg+>Gly>His+>Glu>Lys+>Asp-.

When the method presented herein is used to chemically synthesize a D-amino acids protein, the method mat further include, according to some embodiments thereof, substituting at least one hydrophobic D-amino-acid residue in at least one of the ligation-conducive segments, with a less hydrophobic amino acid, according to the following order of hydrophobicity: D-Ile>D-Leu>D-Phe>D-Val>D-Met>D-Pro>D-Trp>D-His(0)>D-Thr>D-Glu(0)>D-Gln>D-Cys>D-Tyr>D-Ala>D-Ser>D-Asn>D-Asp(0)>D-Arg+>Gly>D-His+>D-Glu>D-Lys+>D-Asp-.

For example, the Pfu-C-4 segment was difficult to synthesize by standard Fmoc-SPPS, with poor solubility in aqueous acetonitrile or 6 M Gn·HCl solutions. It was reckoned that isoleucine is one of the most bulky and hydrophobic proteinogenic amino acids, and thus mutating the isoleucine(s) in a hydrophobic peptide into substituting but potentially less bulky or hydrophobic amino acids (e.g., valine, alanine, leucine, threonine, glycine, phenylalanine, methionine, or proline, etc.), or one or more other bulky or hydrophobic amino acid(s) (such as valine, threonine, phenylalanine, and leucine, etc.) into others that are less bulky or hydrophobic, such as amino acids that are more polar, should alter the physicochemical properties of this peptide segment.

According to some embodiments of the present invention, a systematic isoleucine substitution approach was developed, based on sequence alignment and structural information to mutate all of the seven isoleucine residues in this segment (I598V, I605T, I611V, I619A, I631L, I643V, and I648T) without significantly altering the PCR activity of the polymerase. Indeed, with these seven point mutations, the synthesis of this peptide segment was readily achieved, which also became soluble in aqueous acetonitrile and 6 M Gn·HCl solutions for the downstream purification and NCL, allowing to bypass the need to resort to other chemical modifications for its synthesis.

### Cost Reduction:

In addition to the technical challenges, the synthesis of large mirror-image (D-amino acids) proteins also faces an economic obstacle due to the overall low yield and high reagent cost. While the mirror-image versions of all proteinogenic amino acids are commercially available, most with similar prices as their natural counterparts, D-isoleucine is about 50-to-300-fold more expensive than L-isoleucine and the rest of D-amino acids, mainly due to the existence of two chiral centers that makes its synthesis and purification difficult and lossy, accounting for 80-90% of the D-amino acid cost when synthesizing mirror-image proteins (depending on the abundance of isoleucine in a natural protein, typically at about 5%). Thus, according to some embodiments of the present invention, a systematic isoleucine substitution approach is applied, based on sequence alignment and structural information to mutate a large number (41 out of 71, or 58%) of isoleucines in the Pfu DNA polymerase into other amino acids such as valine, leucine, and alanine, etc., without significantly altering the PCR activity of the polymerase (split Pfu-5m-30I; SEQ ID No. 51).

The systematic Ile-reducing approach resulted in reducing approximately half of the D-amino acid cost for synthesizing this polymerase, which may benefit its large-scale synthesis and applications in the future.

According to some embodiments, the method of chemically producing a D-amino acids protein includes substituting at least one Ile residue with an Ala residue, a Val residue, a Leu residue, a Gly residue, a Thr residue, a Phe residue, a Met residue or a Pro residue. Hence, the resulting D-amino acids protein, some or all the Ile residue positions exhibits a non-Ile D-amino-acid residue selected from the group consisting of a D-Ala residue, a D-Val residue, a D-Leu residue, a Gly residue, a D-Thr residue, a D-Phe residue, a D-Met residue and a D-Pro residue.

### A Method for Total Chemical Synthesis of Large Proteins:

As mentioned hereinabove, and demonstrated in the Examples section that follows below, the total chemical synthesis of a 90-kDa high-fidelity D-amino acid Pfu DNA polymerase was afforded by implementing the method provided herein, and carried out the faithful writing and reading of L-DNA sequences, as well as the accurate assembly of a kilobase-sized mirror-image gene. The average size of natural enzymatic proteins is about 300-500 aa, corresponding to coding gene sequences of about 0.9-1.5 kb. Thus, the ability to synthesize mirror-image versions of enzymatic proteins as large as the Pfu DNA polymerase, and to assemble long mirror-image genes in turn, is a key enabling technology and important stepping stone towards building a mirror-image form of life. From the first-generation mirror-image polymerase ASFV pol X, the second-generation Dpo4, to currently the third-generation Pfu DNA polymerase, with improving technologies, the total chemical synthesis of large mirror-image proteins that exploits the best enzymatic tools that nature offers has become a reality. These efficient next-generation mirror-image enzymes open new doors of opportunity for realizing more sophisticated mirror-image biology systems and expanding the molecular toolbox for biotechnology and medicine.

Thus, according to an aspect of some embodiments of the present invention, there is provided a method for total chemical synthesis of a relatively large and functional protein, which is effected by ligating at least two ligation-conducive segments of the protein, wherein each of the ligation-conducive segments is chemically-synthesizable, or typically about 10-120 amino acid residues long for SPPS; the ligation-conducive segments are obtainable by:
i. identifying at least one ligation-conducive sequence in the amino-acid sequence of the protein; parsing (dividing) the protein's amino-acid sequence at these ligation-conducive sequences, thereby obtaining a plurality of sequences of ligation-conducive segments. According to some embodiments, at least one of the naturally occurring ligation-conducive sequences is found in a structurally-lose section of the protein.
ii. if sequence of the each of the ligation-conducive segments can be effectively synthesized by SPPS and/or AFPS and effectively purified, each of the ligation-conducive segments can be chemically synthesized and be readied for ligation.
iii. if any one of the sequences of the ligation-conducive segments is not chemically-synthesizable, namely longer than about 120, 150 or 200 amino acid residues long, or of other length that cannot be effectively synthesized and purified, these sequences are analyzed for identifying at least one structurally-lose section therein, as this analysis is described hereinabove and known in the art. In order to introduce a ligation-conducive sequence by mutation, at least one amino acid in the structurally-lose section is substituted with a ligation-conducive amino acid residue (e.g., cysteine) so as to introduce a ligation-conducive sequence in the structurally-lose section. Thereafter the amino-acid sequence of the protein is divided (parsed) at this newly introduced ligation-conducive sequence, and the resulting shorter than 120 aa ligation-conducive segments are chemically synthesized.

As discussed hereinabove, exploiting existing, or introducing split sites into the amino acid sequence of the protein, facilitates the total chemical synthesis of the protein. Thus, according to some embodiments of the present invention, the method further includes, prior to Step (i) presented hereinabove, splitting the amino-acid sequence of the protein into at least two domain-forming segments, and if each of the domain-forming segments is chemically-synthesizable (about 120, 150 or 200 amino acid residues long or less), chemically synthesizing each of the domain-forming segments, followed by co-folding these domain-forming segments to thereby obtain the protein.

According to some embodiments, if one of the domain-forming segments is not chemically-synthesizable (e.g., longer than about 120, 150 or 200 amino acid residues), or of other length that cannot be effectively synthesized and purified, it is further divided into ligation-conducive segments, as this is discussed hereinabove.

Preferably, the domain-forming segment is parsed at structurally-lose sections therein, starting with identifying the structurally-lose sections within the domain-forming segment, followed by identifying at least one ligation-conducive sequence in a structurally-lose section, and parsing the amino-acid sequence of the domain-forming segment at these ligation-conducive sequences. Again, if the segment or structurally-lose section is essentially devoid of a ligation-conducive sequence, one can be introduced by mutation, as presented hereinabove. Once the domain-forming segment is parsed into chemically-synthesizable (about 10-120 aa for SPPS, about 10-180 for AFPS) sequences of ligation-conducive segments, the latter are chemically synthesized and ligated to form the domain-forming segment.

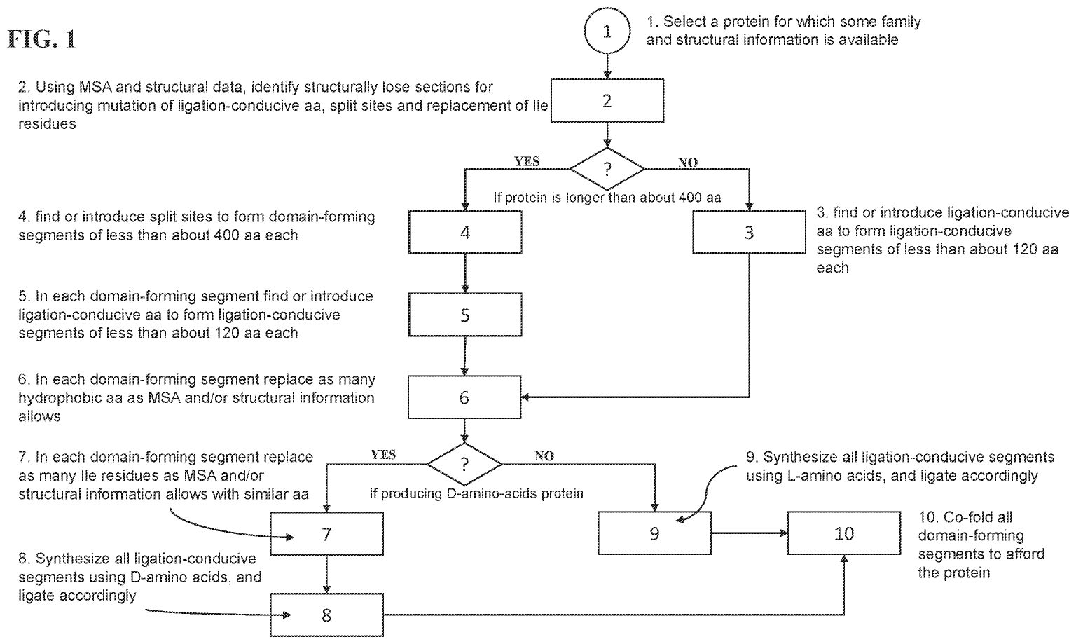

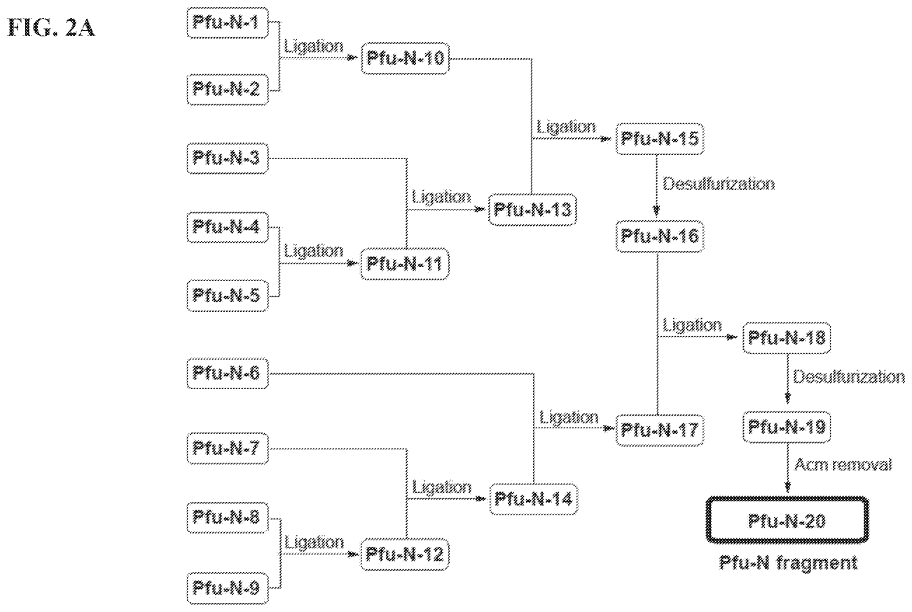

FIG. 1 illustrates the method provided herein in the form of a flowchart, wherein in “Box 1” the user selects a protein of interest, for which preferably some protein family and structural information is available, in “Box 2” the method calls for the use of MSA and structural data to identify structurally-lose sections for introducing mutation of ligation-conducive aa, split sites and replacement of Ile residues; if the protein of interest is shorter than about 400 aa, in “Box 3” the method calls for parsing the sequence of the protein to ligation-conducive segments by finding in and/or introducing ligation-conducive sequences by finding or mutating to ligation-conducive aa, so as to form a plurality of sequences of ligation-conducive segments, each chemically-synthesizable; if the protein of interest is longer than about 400 aa, in “Box 4” the method calls for finding or introducing at least one split site to form domain-forming segments of less than about 400 aa each, and in “Box 5” the method calls for parsing the sequence of each of the domain-forming segments into ligation-conducive segments by finding in and/or introducing ligation-conducive sequences, so as to form a plurality of sequences of ligation-conducive segments, each chemically-synthesizable; in “Box 6” the method calls for replacing hydrophobic aa in each of the domain-forming segments or resulting ligation-conducive segments, based on criteria of sequence preservation according to MSA and/or structural information; if the protein of interest is a D-amino-acids protein, “Box 7” calls for mutating as many Ile residues as MSA and/or structural information allows with similar aa in each domain-forming segment or resulting ligation-conducive segments; and in “Box 8” the method calls for synthesize all ligation-conducive segments using D-amino acids, and ligate the segments accordingly; if the protein of interest is an L-amino-acids protein, “Box 9” calls for synthesizing all ligation-conducive segments using L-amino acids, and ligating the lot accordingly; and finally, in “Box 10”, the method calls for co-folding all domain-forming segments to afford the protein of interest.

In some embodiments of the present invention, the method requires a step of mutating the amino acid sequence of the protein of interest in order to render it suitable for total chemical synthesis. This requirement may be due to excessive length of the protein of interest, in which case the mutations are required in order to introduce a split site that is not present in the corresponding biologically expressed protein, or a ligation-conducive sequences that are not present the corresponding biologically expressed protein, and which are needed to provide ligation-conducive segments that are defined as short enough to be realized by SPPS (or other chemical methods for producing polypeptides). This requirement may be due to excessive hydrophobicity of the ligation-conducive segments, rendering the polypeptides harder to synthesize and ligate under aqueous conditions, whereas lowering their hydrophobicity will render them more suitable for the task.

In some embodiments of the present invention, the method requires a step of mutating the amino acid sequence of the protein of interest in order to render it reduce the cost of total chemical synthesis, particularly when realizing the protein as a D-amino acid protein, namely the mirror-image of its corresponding biologically produced (or expressed) protein, namely the equivalent L-amino acids protein.

In the context of embodiments of the present invention, the terms “corresponding protein”, “corresponding biologically produced protein”, “corresponding biologically expressed protein”, are used interchangeably to refer to the protein which is essentially equivalent to the protein being produced by the herein-provided method in function and to some extent in structure, except for the process of its production, and the amino-acid sequence, that may be mutated in the course of running the herein-provided method, as discussed hereinabove. In the case of mirror-image proteins, the term “corresponding L-amino-acid protein” is similar to the term “corresponding biologically produced protein”, plus the structural inversion compared to the equivalent L-amino-acid protein. Thus, a D-amino acids protein produced by the herein-provided method, relates to its equivalent protein: by having substantially similar sequence, except for: possible mutations to introduce split sites to afford domain-forming segments, and/or possible mutations to introduce ligation-conducive sequences, and/or possible mutations for reducing the hydrophobicity of residues, and/or possible mutations to reduce the number of Ile residues; by having a composition made of at least 90% non-Gly D-amino acid residues rather than L-amino acids residues; by having substantially inverted (mirror-image) structure; and by having similar activity, except for having mirror-image ligands, substrates, products etc. These sequence, composition, structure and activity are present to some extent also between a chemically produced protein, according to some embodiments of the present invention, and its corresponding biologically produced protein, except that the two are made of L-amino acids residues, and thus are not mirror-images of each other in terms of structure and activity.

Part of the method of chemically synthesizing a protein, includes purification and isolation of the resulting protein, after ligation, or after ligation and co-folding of multiple chemically synthesized chains. The purification protocol can be any known protocol for such protein purification task, and in some cases where the target protein is thermostable, the protocol may take advantage of this thermostability in include a heating step, namely the protocol includes a synthesis/ligation steps, followed by a folding step, and further followed by a heat-precipitation step, as part of the purification of the end result. The heat-precipitation temperature is usually set between the maximal stable temperature of the target protein and the minimal precipitation temperature of most of the impurities (incorrectly folded polypeptide chains and polypeptide chains of incorrect amino-acid sequences). For example, in the case of Pfu DNA polymerase, the maximal stable temperature is about 95° C. and the heat-precipitation temperature is therefore set to about 85° C. In the case of Dpo4, the maximal stable temperature is about 86° C., and thus the heat-precipitation temperature is set to about 78° C. The precipitated (thermolabile) impurities are generally removed by ultracentrifugation and/or filtration, while the correctly folded thermostable protein is found in, and can be isolated from the supernatant. It is mentioned herein that multiple folding and heat-precipitation rounds, wherein the proteins precipitated from previous round(s) of folding and heat-precipitation are not discarded, as often done in such procedures, but are rather subjected to additional rounds of re-folding and re-heat-precipitation, are implemented in order to increase the overall yield of correctly folded proteins.

In addition to the above, the scope of the present invention encompasses cases wherein biologically produced proteins and/or protein fragments, are used to induce correct folding of synthetically produced proteins and/or protein fragments. Thus, synthetic proteins and fragments thereof are also afforded, according to some embodiments of the present invention, by co-folding with a biologically produced protein or a fragment thereof, whereas the end result may be a chimeric multi-fragment/domain protein having a biologically produced portion and a synthetically produced portion.

### A Chemically Synthesized Protein:

According to an aspect of some embodiments of the present invention, there is provided a protein, which is chemically synthesized by the method disclosed herein. In some embodiments, the chemically produced protein is at least about 240 amino-acid residues long, or at least about 250 amino-acid residues long, or at least about 300 amino-acid residues long, or at least about 350 amino-acid residues long, or at least about 400 amino-acid residues long, or at least about 450 amino-acid residues long, or at least about 500 amino-acid residues long, or at least about 550 amino-acid residues long, or at least about 600 amino-acid residues long.

The chemically synthesized protein can be any protein of interest, and function as an enzyme, a transport protein, a structure/mechanics protein, a hormone, a signaling protein, an antibody, a fluid-balancing protein, a pH-balancing protein, a cellular channel, or a cellular pump, etc.

The chemically synthesized protein is as functional as its biologically and/or recombinantly produced counterpart, also referred to herein as a corresponding biologically produced protein. The chemically produced protein retains at least 5% of the activity of the corresponding biologically produced protein. In some embodiments, the chemically produced protein retains at least 1%, 5%, 10%, 20%, 30%, 40%, 50%, 60%, 70%, 80% or at least 90% of the activity of the corresponding biologically produced protein.

By retaining at least some percentage of the activity of a corresponding biologically produced protein, it is meant that if a biologically produced protein exhibits a catalytic activity, a specific binding activity, and/or any structurally-related activity, the corresponding chemically produced protein of the present invention exhibits at least 5% of this activity. In cases of a D-amino acids protein, the activity is defined, assessed and measured using the appropriate/corresponding enantiomeric substrates, enantiomeric reactants, enantiomeric reagents and the likes, that correspond to the enantiomeric protein, when compared to its corresponding L-amino acids protein, whether afforded chemically and/or biologically.

According to some embodiments of the present invention, a D-amino acids protein the protein exhibits essentially a mirror-imaged 3D structure compared to the 3D structure of its corresponding biologically produced L-amino acids protein. When producing a D-amino acids protein, also referred to herein as a mirror-image protein (with respect to its corresponding L-amino acids protein, or naturally occurring protein), it is meant that it is produced using at least 75%, 80%, 90% or at least 95% non-Gly D-amino-acid residues in the chemical production of the ligation-conducive segments.

When referring to the protein as comprising at least two domain-forming segments, it is meant that the resulting chemically produced protein, according to embodiments of the present invention, comprises at least two non-covalently attached polypeptide chains (not attached via the main-chain atoms), each corresponding to a domain-forming segment. In some embodiments, the corresponding domain-forming segments are covalently attached polypeptide chains in at least one corresponding family member of the biologically produced protein.

It is noted herein that once a synthetic L-/D-protein is used for any reaction, the reaction mixture can be isolated and synthetic proteins recycled by affinity purification and reused in future reactions, or for its rare and costly amino acid residues. For example, a synthetic protein can be produced with any known affinity tag, such as a His6 tag, and after its use, the reaction mixture can be incubated with the corresponding affinity resin or beads on which the synthetic L-/D-enzyme is isolated from the reaction mixture.

### Exemplary Proteins Prepared by the Method:

According to another aspect of some embodiments of the present invention, there is provided a protein, which is least about 240, 300, 350, 400, 500 or more amino-acid residues long, and produced according to the method provided herein. The protein can be an L-amino acids protein or a D-amino acids protein, depending on the amino acids that are used in the chemical syntheses of the corresponding ligation-conducive segments, e.g., by SPPS.

Tables 1 and 2 below list the genetically encoded amino acids (Table 1) and non-limiting examples of non-conventional/modified amino acids (Table 2) which can be used with the present invention.

TABLE 1

| | Three-Letter | One-letter |
|---|---|---|
| Amino acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane-carboxylate | Cpro | L-N-methylasparagine | Nmasn |
| aminoisobutyric acid | Aib | L-N-methylaspartic acid | Nmasp |
| aminonorbornyl-carboxylate | Norb | L-N-methylcysteine | Nmcys |
| Cyclohexylalanine | Chexa | L-N-methylglutamine | Nmgin |
| Cyclopentylalanine | Cpen | L-N-methylglutamic acid | Nmglu |
| D-alanine | Dal | L-N-methylhistidine | Nmhis |
| D-arginine | Darg | L-N-methylisolleucine | Nmile |
| D-aspartic acid | Dasp | L-N-methylleucine | Nmleu |
| D-cysteine | Dcys | L-N-methyllysine | Nmlys |
| D-glutamine | Dgln | L-N-methylmethionine | Nmmet |
| D-glutamic acid | Dglu | L-N-methylnorleucine | Nmnle |
| D-histidine | Dhis | L-N-methylnorvaline | Nmnva |
| D-isoleucine | Dile | L-N-methylornithine | Nmorn |
| D-leucine | Dleu | L-N-methylphenylalanine | Nmphe |
| D-lysine | Dlys | L-N-methylproline | Nmpro |
| D-methionine | Dmet | L-N-methylserine | Nmser |
| D/L-ornithine | D/Lorn | L-N-methylthreonine | Nmthr |
| D-phenylalanine | Dphe | L-N-methyltryptophan | Nmtrp |
| D-proline | Dpro | L-N-methyltyrosine | Nmtyr |
| D-serine | Dser | L-N-methylvaline | Nmval |
| D-threonine | Dthr | L-N-methylethylglycine | Nmetg |
| D-tryptophan | Dtrp | L-N-methyl-t-butylglycine | Nmtbug |
| D-tyrosine | Dtyr | L-norleucine | Nle |
| D-valine | Dval | L-norvaline | Nva |
| D-α-methylalanine | Dmala | α-methyl-aminoisobutyrate | Maib |
| D-α-methylarginine | Dmarg | α-methyl-γ-aminobutyrate | Mgabu |
| D-α-methylasparagine | Dmasn | α-methylcyclohexylalanine | Mchexa |
| D-α-methylaspartate | Dmasp | α-methylcyclopentylalanine | Mcpen |
| D-α-methylcysteine | Dmcys | α-methyl-α-napthylalanine | Manap |
| D-α-methylglutamine | Dmgln | α-methylpenicillamine | Mpen |
| D-α-methylhistidine | Dmhis | N-(4-aminobutyl)glycine | Nglu |
| D-α-methylisoleucine | Dmile | N-(2-aminoethyl)glycine | Naeg |
| D-α-methylleucine | Dmleu | N-(3-aminopropyl)glycine | Norn |
| D-α-methyllysine | Dmlys | N-amino-a-methylbutyrate | Nmaabu |
| D-α-methylmethionine | Dmmet | α-napthylalanine | Anap |
| D-α-methylornithine | Dmorn | N-benzylglycine | Nphe |
| D-α-methylphenylalanine | Dmphe | N-(2-carbamylethyl)glycine | Ngln |
| D-α-methylproline | Dmpro | N-(carbamylmethyl)glycine | Nasn |
| D-α-methylserine | Dmser | N-(2-carboxyethyl)glycine | Nglu |
| D-α-methylthreonine | Dmthr | N-(carboxymethyl)glycine | Nasp |
| D-α-methyltryptophan | Dmtrp | N-cyclobutylglycine | Ncbut |
| D-α-methyltyrosine | Dmty | N-cycloheptylglycine | Nchep |
| D-α-methylvaline | Dmval | N-cyclohexylglycine | Nchex |
| D-α-methylalnine | Dnmala | N-cyclodecylglycine | Ncdec |
| D-α-methylarginine | Dnmarg | N-cyclododeclglycine | Ncdod |
| D-α-methylasparagine | Dnmasn | N-cyclooctylglycine | Ncoct |
| D-α-methylasparatate | Dnmasp | N-cyclopropylglycine | Ncpro |
| D-α-methylcysteine | Dnmcys | N-cycloundecylglycine | Ncund |
| D-N-methylleucine | Dnmleu | N-(2,2-diphenylethyl)glycine | Nbhm |
| D-N-methyllysine | Dnmlys | N-(3,3-diphenylpropyl)glycine | Nbhe |
| N-methylcyclohexylalanine | Nmchexa | N-(3-indolylyethyl) glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nile | D-N-methylproline | Dnmpro |
| N-(2-methylpropyl)glycine | Nleu | D-N-methylserine | Dnmser |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nva |
| γ-aminobutyric acid | Gabu | N-methyla-napthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| D-N-methylglutamine | Dnmgln | L-α-methylhomo | Mhphe |
| | | phenylalanine | |
| D-N-methylglutamate | Dnmglu | N-(2-methylthioethyl)glycine | Nmet |
| D-N-methylhistidine | Dnmhis | N-(3-guanidinopropyl)glycine | Narg |
| D-N-methylisoleucine | Dnmile | N-(1-hydroxyethyl)glycine | Nthr |
| D-N-methylleucine | Dnmleu | N-(hydroxyethyl)glycine | Nser |
| D-N-methyllysine | Dnmlys | N-(imidazolylethyl)glycine | Nhis |
| N-methylcyclohexylalanine | Nmchexa | N-(3-indolylyethyl)glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nleu | D-N-methylproline | Dnmpro |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nval |
| γ-aminobutyric acid | Gabu | N-methyla-napthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| L-α-methylleucine | Mleu | L-α- | Mhphe |
| | | methylhomophenylalanine | |
| L-α-methylmethionine | Mmet | N-(2-methylthioethyl)glycine | Nmet |
| L-α-methylnorvaline | Mnva | L-α-methyllysine | Mlys |
| L-α-methylphenylalanine | Mphe | L-α-methylnorleucine | Mnle |
| L-α-methylserine | mser | L-α-methylornithine | Morn |
| L-α-methylvaline | Mtrp | L-α-methylproline | Mpro |
| L-α-methylleucine | Mval | L-α-methylthreonine | Mthr |
| | Nnbhm | | |
| N-(N-(2,2- | Nnbhm | L-α-methyltyrosine | Mtyr |
| diphenylethyl)carbamylmethyl- | | | |
| glycine | | | |
| 1-carboxy-1-(2,2-diphenyl | Nmbc | L-N- | Nmhphe |
| ethylamino)cyclopropane | | methylhomophenylalanine | |
| N-(N-(3,3- | Nnbhe | D/L-citrulline | D/Lctr |
| diphenylpropyl)carbamylmethyl(1)glycine | | | |

In order to demonstrate the method of total chemical synthesis of proteins, the present inventors synthesized active enzymes that are capable of catalyzing a reaction catalyzed by their corresponding biologically produced enzymes. One of these enzymes is an RNA polymerase, capable of synthesizing RNA from ribonucleotides using a DNA template. In the Examples section that follows below, the exemplary RNA polymerase is a T7 RNA polymerase. In another example, the enzyme is a DNA polymerase, which is capable of synthesizing DNA from deoxyribonucleotides. In the Examples section that follows below, the exemplary DNA polymerase is a Pfu DNA polymerase.

When the method provided herein is used to produce a D-amino acids RNA polymerase, this unique mirror-image enzyme is capable of synthesizing L-RNA from L-ribonucleotides using an L-DNA template. For example, the D-amino acids RNA polymerase is a D-amino acids T7 RNA polymerase.

As presented hereinbelow, the D-amino acids T7 RNA polymerase is prepared with at least one split site, a first split site between K363 and P364 and a second split site between N601 and T602, using the WT position numbering scheme. Alternatively, the D-amino acids T7 RNA polymerase, as well as the L-amino acids T7 RNA polymerase produced by the herein-provided method, include at least two polypeptide chains formed by a split between K363 and P364 and/or a split between N601 and T602. Furthermore, the said split site can be potentially chosen near the above-mentioned sites in the same loop, namely from position 357 to position 366 and/or from position 564 to position 607.

According to some embodiments of the present invention, a T7 RNA polymerase produced according to the herein-provided method, may further include at least one mutation selected from the group consisting of I6V, I14L, I74V, I82V, I109V, I117L, I141V, I210M, I244L, I281V, I320V, I322L, I330V and I367L. These mutations are conducive with the cost-reduction strategy, by replacing the costly D-Ile residue with another compatible D-amino acid residue.

According to an aspect of the present invention, there is provided a D- or an L-amino acids T7 RNA polymerase, produced by the herein-provided method, is having an amino-acid sequence identical to SEQ ID No. 83, or having at least 80-90% sequence identity to SEQ ID No. 83.

When the method provided herein is used to produce a D-amino acids DNA polymerase, this unique mirror-image enzyme is capable of synthesizing L-DNA from L-deoxyribonucleotides. For example, the D-amino acids DNA polymerase is a D-amino acids Pfu DNA polymerase.

Thus, according to another aspect of the present invention, there is provided a Pfu DNA polymerase, that includes at least two polypeptide chains formed by a split between K467 and M468, whereas position numbering is based on the amino acid position numbering of the corresponding WT enzyme. It is noted herein that other split sites may be selected near this site, i.e., in the coiled-coil motif of the fingers domain of the Pfu DNA polymerase, for example, between position 449 and position 498.

According to some embodiments, the synthetic Pfu DNA polymerase provided herein further includes at least one mutation selected from the group consisting of E102A, E276A, K317G, V367L and 1540A. According to other embodiments, the Pfu DNA polymerase provided herein further comprising at least one mutation selected from the group consisting of V93Q, D141A, E143A, Y410G, A486L and E665K.

According to an aspect of the present invention, there is provided a D- or an L-amino acids Pfu DNA polymerase, with or without DNA binding structural domain (SEQ ID No. 78), produced by the herein-provided method, is having an amino-acid sequence selected form the group consisting of SEQ ID No. 48, SEQ ID No. 49, SEQ ID No. 50, SEQ ID No. 51, SEQ ID No. 74, SEQ ID No. 75, SEQ ID No. 76, SEQ ID No. 77, and SEQ ID No. 79, or having at least 80-90% sequence identity to SEQ ID No. 51.

### Bioorthogonal Data Storage:

The increasingly rapid pace at which data are being generated worldwide has created a growing need for reliable, high-density media to preserve the massive information. Natural DNA is exquisitely evolved to encode, store, and propagate information.

Storage in DNA, nature's molecule of choice for encoding vast genomic instructions in tightly packed chromosomes, has emerged as a promising solution (1-3). On the other hand, mirror-image DNA is uniquely suited for the task of bioorthogonal information storage, for which purpose the methodology of L-DNA data deposition and retrieval is essential but has remained largely unexplored.

The present inventors have contemplated that chirally inverted (mirror-image) DNA, which possesses the same informational capacity, holds unique abilities to evade biological degradation and contamination, and may therefore serve as a highly robust, bioorthogonal data repository. While reducing the present invention to practice, a 90-kDa high-fidelity D-amino acid Pfu DNA polymerase has been chemically synthesized, according to some embodiments of the present invention, for the faithful writing and reading of L-DNA sequences.

The present inventors have demonstrated one of the aspect of some embodiments of the present invention—the storage of an entire paragraph of digital text in mirror-image DNA. As can be seen in the Example section that follows below, the trace message-carrying L-DNA barcode in unpurified environmental water samples remained stable and amplifiable for months and potentially beyond. Moreover, the high-fidelity D-polymerase, produced according to some embodiments of the present invention, enabled the accurate assembly of a full-length kilobase-sized mirror-image gene, an imperative step towards achieving mirror-image translation and establishing the mirror-image central dogma. The successful synthesis of next-generation mirror-image enzymatic tools and, in turn, assembly of long mirror-image genes, transformed the development of mirror-image biology systems and exploration of their emerging applications.

Briefly, DNA is essentially a data storage molecule. It contains all of the instructions a cell (or an entire organism) needs to sustain itself. These instructions are found within genes, which are sections of DNA made up of specific sequences of nucleotides. In order to be implemented, the instructions contained within genes must be expressed, or copied into a form that can be used by cells to produce the proteins needed to support life. The instructions stored within DNA are read and processed by a cell in two steps: transcription and translation. Each of these steps is a separate biochemical process involving multiple molecules. During transcription, a portion of the cell's DNA serves as a template for creation of an RNA molecule. In some cases, the newly created RNA molecule is itself a finished product, and it serves an important function within the cell. In other cases, the RNA molecule carries messages from the DNA to other parts of the cell for processing. Most often, this information is used to manufacture proteins. The specific type of RNA that carries the information stored in DNA to other areas of the cell is called messenger RNA, or mRNA.

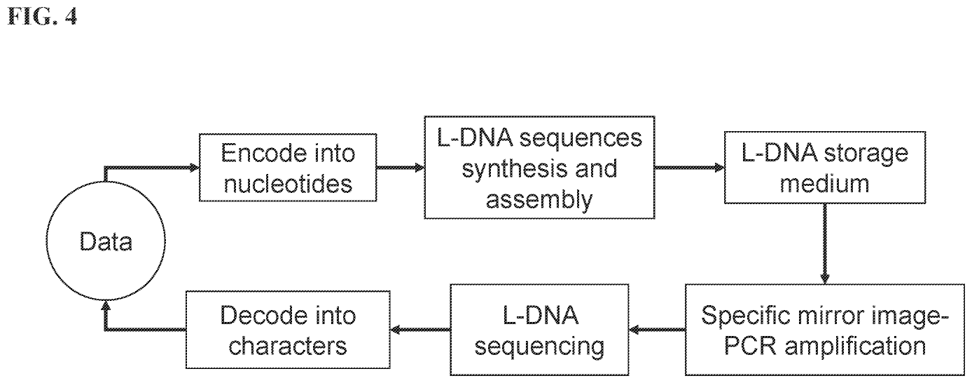

FIG. 4 is a flowchart illustrating molecular data storage, according to some embodiments of the present invention, using L-DNA as an exemplary type of XNA.

Thus, according to an aspect of embodiments of the present invention, there is provided a method of forming a biorthogonal data storage polymer, using a D-amino acids RNA polymerase or a D-amino acids DNA polymerase, and L-ribonucleic acids or L-deoxyribonucleic acids, respectively, wherein said polymerase is produced according to the method provided herein.

According to another aspect of embodiments of the present invention, there is provided a method of forming a biorthogonal data storage polymer, using the herein-provided D-amino acids RNA polymerase or the herein-provided D-amino acids DNA polymerase, and L-ribonucleic acids or L-deoxyribonucleic acids, respectively.

According to another aspect of embodiments of the present invention, there is provided a method of decoding a biorthogonal data storage polymer, using at least one D-amino acids protein produced by the herein-provided method, wherein the biorthogonal data storage polymer comprises L-ribonucleic acids or L-deoxyribonucleic acid residues.

According to yet another aspect of embodiments of the present invention, there is provided a biorthogonal data storage system, comprising at least one L-DNA that encodes for the information data in its sequence, using the four characters A, T, G and C, a D-amino acids RNA/DNA polymerase for synthesizing the L-DNA (writing the code into the DNA sequence), and/or for sequencing (reading the code in the DNA sequence) the L-DNA, essentially as described in the foregoing.

It is noted herein that the scope of the present invention is intended to encompass the use of other types of non-naturally occurring or non-canonical nucleotides and polymers thereof, referred to herein and in the art as “Xeno Nucleic Acid”, or XNAs. Thus, according to some embodiments of the present invention, the systems and methods provided here for producing and using molecular data storage, include the use of XNAs, such as those discussed, for example, by Eremeeva, E and Herdewijn, P. in the publication “Non canonical genetic material” [Current Opinion in Biotechnology, 2019, 57, pp. 25-33], and by Chaput, J. C. et al. [Chem. Biol., 2012, 21; 19(11), pp. 1360-71].

The faithful assembly, amplification, and sequencing of L-DNA may present exciting opportunities for bioorthogonal information storage, environmental and food barcoding, medical implant monitoring, forensic investigation, as well as secure messaging, which were not realized by the earlier versions of mirror-image polymerase systems such as ASFV pol X or Dpo4 because they were too inefficient and error-prone for the amplification and sequencing of a small amount of information-bearing L-DNA molecules (5, 17, 18, 21). The accurate assembly of mirror-image genes and even entire genomes in the future could also make the system suitable for producing mirror-image genome backup copies of natural organisms for genome banking and interplanetary transportation purposes.

### Mirror-Image Ribosome:

The next step in establishing the mirror-image central dogma is to achieve mirror-image translation through building a functional mirror-image ribosome. Although the present inventors have recently overcome the limitations of L-RNA chemical synthesis (typically less than about 70 nt) by transcribing a synthetic L-DNA template into full-length 5S rRNA at 120 nt, more efficient enzymatic systems capable of transcribing mirror-image genes into longer L-RNAs are required for obtaining the 1.5-kb 16S and 2.9-kb 23S rRNAs, as well as mRNAs for translation. One possibility is to mutate DNA polymerases into DNA-dependent RNA polymerases as previously demonstrated. Indeed, the present inventors have succeeded in reengineering the split Pfu DNA polymerase (with seven point mutations V93Q, E102A, D141A, E143A, Y410G, A486L, and E665K) into an efficient DNA-dependent RNA polymerase. However, the preparation and purification of long single-stranded (ss) L-DNA templates poses another challenge and should be addressed first. Alternatively, synthesizing the mirror-image version of the 100-kDa T7 RNA polymerase which uses double-stranded (ds) L-DNA templates should enable the enzymatic transcription of all the mirror-image rRNAs and mRNAs needed for mirror-image translation. In the process of reducing the present invention to practice, D-amino acids T7 RNA polymerase was realized by total chemical synthesis, according to some embodiments of the present invention, as presented in the Examples section that follows below.

### Racemic Crystallography:

As known in the art of protein crystallography, the first and probably the most rate-limiting step in protein structure elucidation is obtaining X-ray diffraction-capable crystals. It has been observed in small molecules crystallization experiments, which racemic mixtures of two enantiomers of a molecule tend to form high-quality diffracting crystals, wherein at least one of the symmetric operations observed in the unit cell is inversion. The emerging field of racemic crystallography in structural biology suffers from lack of mirror image protein samples, due to their scarcity, particularly when seeking large mirror image proteins.

Thus, according to some embodiments of the present invention, there is provided a method for forming a crystal of a protein of interest, which is effected by co-crystallizing the protein of interest and an enantiomorph of that protein of interest, which is afforded as provided herein, thereby forming a crystal of an enantiomeric protein pair, wherein the enantiomorph is the D-amino-acids (mirror image) protein and the corresponding L-amino acids protein of interest.

In another type of embodiments of the present invention, the mirror image enantiomorph is produced by a mirror image protein, as provided herein. For example, a mirror-image high-fidelity RNA polymerase, provided as discussed herein, can be used for transcribing L-RNA, thereby produce the enantiomorph of its corresponding D-RNA, which can then be used for enantiomeric/racemic co-crystallization with D-RNA for solving RNA structures.

Additional information pertaining to racemic crystallography, can be found, for example, in: Matthews, B. W., “Racemic crystallography-Easy crystals and easy structures: What's not to like?”, Protein Science, 2009, 18(6), pp. 1135-1138; Yeates, T. O. and Kent, S. B. H., “Racemic Protein Crystallography”, Annual Review of Biophysics, 2012, 41(1), pp. 41-61; and Mandal, P. K. et al., “Racemic DNA Crystallography”, Angewandte Chemie International Edition, 2014, 53(52), pp. 14424-14427, the contents of which is incorporated herewith by reference in its entirety as if fully set forth herein.

### Sequencing:

According to some embodiment of the present invention, the synthetic proteins can be used for sequencing, and denaturing sequencing PAGE for separation of chemically synthesized mirror-image DNA oligos to substantially improve the quality of synthetic oligos by reducing the vast majority of the −1 and −2 nt products. This use of either D- or L-amino acid synthetic protein improves the fidelity of the sequencing process, such that the majority of the final assembled gene sequences are of correct sequence.

According to some embodiments of the present invention, unlabeled carrier D- (or L-) DNA is added to the samples prior to purification by denaturing sequencing PAGE (which has a certain required amount as its “dead volume”), in order to reduce the required scale of mirror-image-PCR and PCR-amplified L-DNA products for the gel purification. According to some embodiment of the present invention, the synthetic mirror-image high-fidelity polymerase, can be used with phosphorothioate L-dNTPs for sequencing-by-synthesis of mirror-image nucleic acids such as L-DNA and L-RNA. Also, use of a bi-directional sequencing strategy by 5′-labelled two primers with two different dyes (FAM and Cy5, respectively) is used to improve the read length in one reaction to >160 to 170 bp.

### Systematic Evolution of Ligands by Exponential Enrichment:

The development of sequencing-by-synthesis, for example using the mirror-image Pfu DNA polymerase provided herein, according to some embodiments of the present invention, is another step forward towards realizing more effective L-DNA sequencing techniques compared with the cumbersome L-DNA chemical sequencing approach.

Systematic evolution of ligands by exponential enrichment (SELEX), also referred to as in vitro selection or in vitro evolution, is a combinatorial chemistry technique in molecular biology for producing oligonucleotides of either single-stranded DNA or RNA that specifically bind to a target ligand or ligands. The process begins with the synthesis of a large oligonucleotide library consisting of randomly generated sequences of fixed length flanked by constant 5′ and 3′ ends that serve as primers. For a randomly generated region of length n, the number of possible sequences in the library is 4n (n positions with four possibilities (A, T, C, and G) at each position). The sequences in the library are exposed to the target ligand—which may be a protein or a small organic compound—and those that do not bind the target are removed, usually by affinity chromatography or target capture on paramagnetic beads. The bound sequences are eluted and amplified by PCR to prepare for subsequent rounds of selection in which the stringency of the elution conditions can be increased to identify the tightest-binding sequences. SELEX has been used to develop a number of aptamers that bind targets interesting for both clinical and research purposes. Also towards these ends, a number of nucleotides with chemically modified sugars and bases have been incorporated into SELEX reactions. These modified nucleotides allow for the selection of aptamers with novel binding properties and potentially improved stability.

Future efforts to reengineer the high-fidelity mirror-image polymerase (e.g., through synthesizing mutant or truncated versions without 3′-5′ exonuclease activity) for mirror-image Sanger sequencing and even automated, high-throughput L-DNA sequencing techniques may lead to new applications such as multiplexed L-DNA sequencing, and mirror-image Systematic Evolution of Ligands by Exponential Enrichment (MI-SELEX) for the direct in vitro selection of L-aptamer drugs (17, 18).

It is expected that during the life of a patent maturing from this application many relevant large synthetic D/L-proteins will be developed and the scope of the term large synthetic D/L-proteins is intended to include all such new technologies a priori.

As used herein the term “about” refers to ±10% (e.g., “about 30” means 27-33 or 30±3).

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Total Chemical Synthesis of Pfu DNA Polymerase

A proof of concept of some embodiments of the present invention was carried out by the total chemical synthesis of both the natural (L-amino acids protein) and mirror-image versions of the Pfu DNA polymerase.

The first step in implementing the method provided herein, was to use the available information pertaining to Pfu DNA polymerase, in order to identify the existing sequence features that are conducive to total chemical synthesis of the enzyme, and the identify locations in the sequence with sufficient structural flexibility (looseness) to allow introducing mutation therein without compromising the structural stability, and thus the desired activity of the enzyme. To that end, a multiple sequence alignment (MSA) was performed using Pfu-WT (SEQ ID No. 47), Pfu-5m (SEQ ID No. 48), Pfu-5m-55I (SEQ ID No. 49), Pfu-5m-46I (SEQ ID No. 50), Pfu-5m-30I (SEQ ID No. 51), Pfu-5m-0I (SEQ ID No. 52), KOD1 (SEQ ID No. 53), Tgo (SEQ ID No. 54), 9° N-7 (SEQ ID No. 55), and Tok (SEQ ID No. 56) polymerases. The MSA reviled the highly conserved amino acids, which were kept unchanged, while other parts on the MSA showed diversity conducive to mutations for introducing therein additional NCL sites, split sites, hydrophobicity-lowering mutations and Ile-reducing mutations. Thus, based on the MSA, E102A, E276A, K317G, V367L and I540A were chosen as mutations for introducing ligation-conducive amino acids in diverse amino-acid sections of the sequence (as well as replacing the isoleucine at position 540). Based on the MSA analysis and protein structural information, isoleucine WT residues I38, I62, I65, I80, I127, I137, I158, I171, I176, I191, I197, I198, I205, I206, I228, I232, I244, I256, I264, I268, I282, I331, I401, I434, I446, I478, I557, I598, I605, I611, I619, I631, I643, I648, I656, I677, I716, I734, I745 and I772 were replaced with other compatible residues. In addition, the V93Q, D141A, E143A, Y410G, A486L and E665K mutations were introduced in order to turn the Pfu DNA polymerase into an efficient RNA polymerase in both the L- and the D-amino acids versions.

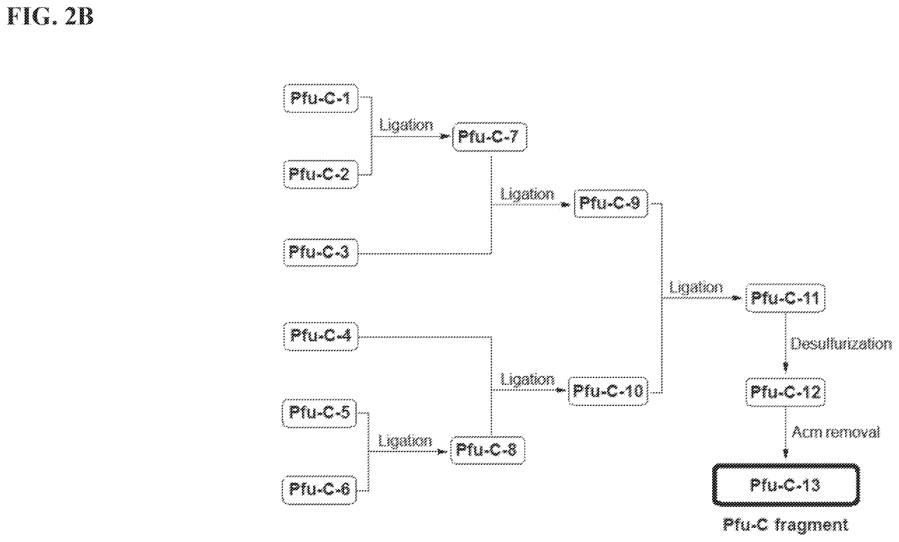

The amino-acid sequence of Pfu DNA polymerase was split into two domain-forming segments, according to some embodiments of the present invention, referred to herein as the Pfu-N fragment (SEQ ID No. 57) and the Pfu-C fragment (SEQ ID No. 67). Pfu-N fragment was divided into 9 peptide segments ranging from 40 to 62 aa in lengths (SEQ ID Nos. 58-66), and the Pfu-C fragment was divided into 6 segments ranging from 33 to 63 aa (SEQ ID Nos. 68-73), as seen in FIGS. 2A-B below.

FIGS. 2A-B present the design flow of the synthetic route of the mutant Pfu-N fragment (FIG. 2A), wherein additional NCL sites were introduced (E102A, E276A, K317G, V367L) to form ligation-conducive segments, and 25 isoleucine residues were substituted, and the design flow of the synthetic route of the mutant Pfu-C fragment (FIG. 2B), wherein an additional NCL site (I540A) was introduced, as well as the mutation of other 15 isoleucine residues, whereas these mutations were introduced to facilitate protein synthesis in SPPS and ligation process and reduce synthesis cost of the mirror-image version.

The peptide segments were prepared by Fmoc-based SPPS, purified by reversed-phase high-performance liquid chromatography (RP-HPLC), and assembled by hydrazide-based NCL with a convergent assembly strategy, followed by metal-free radical-based desulfurization. 4.3 mg L-Pfu-N fragment were obtained with an observed molecular weight (M.W.) at 54830.0 Da (calculated M.W. 54829.9 Da; as determined by analytical HPLC and ESI-MS, not shown) and 2.2 mg L-Pfu-C fragment with an observed M.W. at 35563.2 Da (calculated M.W. 35563.02 Da) for the L-polymerase, 16.5 mg D-Pfu-N fragment with an observed M.W. at 54829.5 Da and 11.9 mg D-Pfu-C fragment with an observed M.W. at 35561.9 Da for the D-polymerase. Both the synthetic L-polymerases and D-polymerases were folded by successive dialysis, followed by heat-precipitation at 85° C., which further improved the purity of the correctly folded protein (ESI-MS, not shown). Next, the PCR activity of the polymerases was tested on short, 100-bp synthetic D- or L-DNA templates (SEQ ID No. 12), and measured comparable amplification efficiencies between recombinant, and synthetic L-polymerase and D-polymerase (analyzed by 3% sieving agarose gel electrophoresis and stained by ExRed. M, DNA marker, and ImageLab software (Bio-Rad Laboratories, CA, U.S.). M, DNA marker). The fidelity of the synthetic L-polymerase was also quantified on a 1.2-kb D-DNA sequence from the pUC19 plasmid (SEQ ID No. 80), and Sanger sequencing of the PCR products measured an error rate of less than 3.6×10⁻⁶ (see, Table 3 below), consistent with that of the WT Pfu DNA polymerase reported in previous studies.

TABLE 3

| | | | | | Oligo | |
|---|---|---|---|---|---|---|
| | | | | Total | purifi- | |
| | Dele- | Inser- | Substi- | sequenced | cation | Error |
| Procedure | tion | tion | tution | bases | method | rate |
| Polymerization | 0 | 0 | 4 | 91728 | — | 3.6 × 10⁻⁶ |
| (35-cycle PCR) | | | | | | |
| Gene assembly | 28 | 0 | 2 | 10661 | OPC | 2.8 × 10⁻³ |
| Gene assembly | 0 | 0 | 1 | 15230 | PAGE | 6.6 × 10⁻⁵ |

### Materials:

L-DNA oligos were synthesized on the H-8 oligo synthesizer (K&A Laborgeraete, Germany) with L-deoxynucleoside phosphoramidites (ChemGenes, MA, U.S.). Primers for recombinant protein expression were ordered from Genewiz (Beijing, China). Primers for bacterial 16S rRNA gene assembly were purified by denaturing sequencing PAGE. Other DNA oligos were purified by oligonucleotide purification cartridges (OPC) (Ruibiotech, Beijing, China). The PAGE DNA Purification Kit was purchased from Tiandz Inc. (Beijing, China). Tris-base, NP-40, Tween-20, KCl, guanidine hydrochloride (Gn·HCl), and β-mercaptoethanol (β-ME) were purchased from Amresco Inc. (PA, U.S.). Imidazole and EDTA were purchased from Solarbio Life Sciences (Beijing, China). 2-Chlorotrityl Chloride Resin (loading=0.6 mmole/g) was purchased from Tianjin Nankai Hecheng Science & Technology Co. (Tianjin, China). Wang Chemmatrix resin was purchased from CSBio Ltd (Shanghai, China). Fmoc-D-amino acids, Fmoc-L-amino acids, and O-(6-chlorobenzotriazol-1-yl)-N,N,N′,N′-tetramethyluronium hexafluorophosphate (HCTU) were purchased from GL Biochem Co. (Shanghai, China). N,N-Diisopropylethylamine (DIEA), trifluoroacetic acid (TFA), N,N-dimethylformamide (DMF), thioanisole, triisopropylsilane (TIPS), 1,2-ethanedithiol (EDT), palladium chloride (PdCl₂), sodium 2-mercaptoethanesulfonate (MESNa), and 2,2′-azobis [2-(2-imidazolin-2-yl)propane] dihydrochloride (VA-044) were purchased from J&K Scientific (Beijing, China). 4-Mercaptophenylacetic acid (MPAA) was purchased from Alfa Aesar Chemicals Co. (Shanghai, China). Piperidine, Na₂HPO₄·12H₂O, NaH₂PO₄·2H₂O, sodium nitrite (NaNO₂), and acetic anhydride were purchased from Sinopharm Chemical Reagent Co. (Shanghai, China). NaCl, NaOH, and hydrochloric acid were purchased from Sinopharm Chemical Reagent (Beijing, China). Dichloromethane (DCM) was purchased from Shanghai Titan Scientific Co. (Shanghai, China). Tris (2-carboxyethyl) phosphine hydrochloride (TCEP·HCl), 9-fluorenylmethyl carbazate (Fmoc-NHNH₂), ethyl cyanoglyoxylate-2-oxime (Oxyma), N,N′-diisopropylcarbodiimide (DIC), and DL-1,4-dithiothreitol (DTT) were purchased from Adamas Reagent Co. (Shanghai, China). Glutathione reduced (GSH) was purchased from Acros Organics (NJ, U.S.). Anhydrous ether was purchased from Beijing Tongguang Fine Chemicals Company (Beijing, China). Acetonitrile (HPLC grade) was purchase from J. T. Baker (NJ, U.S.).

### Fmoc-Based Solid-Phase Peptide Synthesis (Fmoc-SPPS):

All peptides were synthesized by Fmoc-based SPPS on Liberty Blue automated microwave peptide synthesizer (CEM Corporation, NC, U.S.) and Prelude X automated peptide synthesizer (Protein Technologies Inc., AZ, U.S.). Peptides with C-terminal carboxylate such as Pfu-N-9 and Pfu-C-6 were synthesized on Wang Chemmatrix resin (CSBio Ltd, Shanghai, China) preloaded with the first C-terminal residue. All the other peptides were synthesized on Fmoc-hydrazine 2-chlorotrityl chloride resin to prepare peptide hydrazides. For each peptide acid, the first residue was manually attached to the Wang Chemmatrix resin by a double coupling method: in the first coupling reaction, amino acid was coupled for 1 h at 30° C. using 4 equiv. amino acid, 3.8 equiv. HCTU, and 8 equiv. DIEA, and the resin was washed with DMF and DCM; without deprotection, the second coupling reaction was carried out overnight at 25° C. with 4 equiv. amino acid, 4 equiv. Oxyma, and 4 equiv. DIC. All resins were swelled in DMF for 5-10 min before use. The Fmoc groups of both resins and the assembled amino acids were removed by treatment with 20% piperidine and 0.1 mol/L Oxyma in DMF at 85° C. Coupling of amino acids except Fmoc-Cys(Trt)-OH and Fmoc-His(Trt)-OH was carried out at 85° C. using 4 equiv. amino acid, 4 equiv. Oxyma, and 8 equiv. DIC. The coupling reactions for Fmoc-Cys(Trt)-OH and Fmoc-His(Trt)-OH were carried out at 50° C. for 10 min to avoid side reactions at high temperature. Trifluoroacetyl thiazolidine-4-caboxylic acid-OH (Tfa-Thz-OH) was coupled using Oxyma/DIC activation at room temperature. After the completion of peptide chain assembly, peptides were cleaved from resin using H₂O/thioanisole/triisopropylsilane/1,2-ethanedithiol/trifluoroacetic acid (0.5/0.5/0.5/0.25/8.25). The cleavage reaction took 2.5 h under agitation at 27° C. Most of the TFA in the mixture was removed by N2 blowing, and cold ether was added to precipitate the crude peptide. After centrifugation, the supernatant was discarded and the precipitates were washed twice with ether. The crude peptides were dissolved in CH₃CN/H₂O, analyzed by RP-HPLC and ESI-MS, and purified by semi-preparative HPLC.

### Native Chemical Ligation (NCL):

C-terminal peptide hydrazide segment was dissolved in acidified ligation buffer (aqueous solution of 6 M Gn·HCl and 0.1 M NaH₂PO₄, pH 3.0). The mixture was cooled in an ice-salt bath (−10° C.), and 10 eq. NaNO₂ in acidified ligation buffer (pH 3.0) was added. The activation reaction system was kept in ice-salt bath under stirring for 25 min, after which 40 eq. MPAA in ligation buffer and 1 eq. N-terminal cysteine peptide were added, and the pH of the solution was adjusted to 6.5 at room temperature. After overnight reaction, 150 mM TCEP in ligation buffer (pH adjusted to 7.0) was added to dilute the system twice and the reaction system was kept at room temperature for 30 min under stirring. Finally, the ligation product was analyzed by HPLC and ESI-MS, and purified by semi-preparative HPLC. Notably, during the ligation of the Pfu-C-1 and Pfu-C-2 segments, it was discovered that the ligation was very inefficient due to the insoluble Pfu-C-2 segment, and thus the initial concentration of Gn·HCl was increased to 8 M (final Gn·HCl concentration at about 7 M), which significantly improved solubility and ligation efficiency of the two peptide segments.

### Desulfurization:

Cys-containing peptide (3 mg/ml) was dissolved in desulfurization buffer (0.1 M aqueous phosphate buffer containing 6 M Gn·HCl, 200 mM TCEP, 40 mM reduced L-glutathione and 20 mM VA-044, pH 6.8). The mixture was under stirring at 37° C. overnight, and the desulfurization product was analyzed by HPLC and ESI-MS, and purified by semi-preparative HPLC.

### Acm Deprotection:

Acetamidomethyl (Acm) group was removed by the Pd-assisted deprotection strategy. Acm-protected peptide was dissolved in Acm deprotection buffer (aqueous solution of 6 M Gn·HCl, 0.1 M phosphate and 40 mM TCEP, pH 7.0) to a final concentration of 1 mM, after which 20 eq. PdCl₂ was added. The reaction mixture was incubated with agitation at 25° C. overnight. DTT was added to 50 mM final concentration to quench the reaction. The reaction mixture was under stirring for 1 h and purified by semi-preparative HPLC.

### Folding of Split Pfu DNA Polymerases In Vitro:

Lyophilized N fragment and C fragment of Pfu DNA polymerase were dissolved in 4 M and 5 M Gn·HCl containing 10 mM β-ME, respectively. Protein folding in vitro was performed by mixing equal concentrations of the two fragments (0.5 μM), followed by dialyzing against a buffer containing 40 mM Tris-HCl (pH 7.5), 1 mM EDTA, 100 mM KCl, 10% glycerol, overnight at 4° C. The folded Pfu DNA polymerase was heated to 85° C. for 15 min to precipitate thermolabile peptides, which were subsequently removed by centrifugation at 20,000×g for 40 min at 4° C. The supernatant was concentrated and dialyzed against a storage buffer 100 mM Tris-HCl (pH 8.0), 50% glycerol, 0.2 mM EDTA, 0.2% NP-40 nonionic detergent, 0.2% Tween 20, 2 mM DTT.

### RP-HPLC and ESI-MS:

All RP-HPLC analyses and purifications were carried out on Shimadzu Prominence HPLC systems (Shimadzu, Kyoto, Japan) with SPD-20A UV-Vis detectors and LC-20AT solvent delivery units. Ultimate XB-C4 column (5 μm, 4.6×250 mm) (Welch Materials, Shanghai, China) was used for analysis at a flow rate of 1 ml/min to monitor the ligation reactions and analyze the purity of the peptide products. Ultimate XB-C4 and C18 column (5 μm, 21.2×250 mm or 5 μm, 10×250 mm) (Welch Materials, Shanghai, China) were used to separate the crude peptides and ligation products, respectively, at a flow rate of 4-8 ml/min. The purified products were characterized by ESI-MS on a Shimadzu LC/MS-2020 system (Shimadzu, Kyoto, Japan).

### Protein Expression and Purification:

The gene of Pfu DNA polymerase was cloned into the pET-28c plasmid, and mutants were constructed by the pEASY-Uni Seamless Cloning and Assembly Kit (TransGen Biotech., Beijing, China). Proteins fused to an N-terminal His6 tag were expressed using E. coli strain BL21 (DE3) in LB medium. The induced cells were harvested and resuspended in lysis buffer (40 mM Tris-HCl, 300 mM NaCl, 10 mM imidazole, 10 mM β-ME, 10 mg/ml lysozyme, pH 8.0). Cell lysate was heated at 85° C. for 15 min, and the thermolabile proteins were subsequently removed by centrifugation at 20,000×g for 40 min at 4° C. The supernatant was incubated in Ni-NTA Superflow resin (Senhui Microsphere Tech., Suzhou, China) for 1h at 4° C. The resin was washed by a buffer containing 40 mM Tris-HCl (pH 8.0), 300 mM NaCl, 40 mM imidazole, and 10 mM β-ME, which was then eluted by a buffer containing 40 mM Tris-HCl (pH 8.0), 300 mM NaCl, 250 mM imidazole, and 10 mM β-ME. The purified and concentrated Pfu DNA polymerse and mutants were dialyzed against a storage buffer containing 100 mM Tris-HCl (pH 8.0), 50% glycerol, 0.2 mM EDTA, 0.2% NP-40 nonionic detergent, 0.2% Tween 20, and 2 mM DTT.

### PCR Activity and Fidelity:

The natural and mirror-image PCR reactions were performed in 50 μl reaction system containing 1× Pfu buffer (Solarbio Life Sciences, Beijing, China), with 200 μM (each) dNTPs, 0.2 μM (each) primers, template, and polymerase. To quantify the PCR activity of Pfu DNA polymerase and its mutants, the polymerases were adjusted to the same concentration with wild-type (WT) Pfu DNA polymerase by 12% SDS-PAGE. An SDS-PAGE analysis confirmed the molecular weight similarity of the fragments of the recombinant split, mutant Pfu DNA polymerase expressed and purified from E. coli, and the synthetic natural and mirror-image Pfu DNA polymerases of the same sequence (results not shown). The PCR program settings were 94° C. for 3 min (initial denaturation); 94° C. for 30 s, 50-65° C. (Tm-dependent) for 30 s, and 72° C. for 1-7 min (depending on the amplicon length), for 10-35 cycles; 72° C. for 10 min (final extension). To quantify the amplification efficiency of synthetic Pfu DNA polymerase, a 100-bp DNA sequence was used as template. PCR amplification by recombinant, synthetic L- and synthetic D-Pfu DNA polymerase (split Pfu-5m-30I) were analyzed by 3% sieving agarose gel electrophoresis and stained by ExRed (results not shown). The PCR amplification efficiency of the synthetic D-Pfu DNA polymerase measured about 1.5, estimated based on the intensity of the product bands. The amplification products of the first 9 cycles were analyzed by the ImageJ software (Bio-Rad Laboratories, CA, USA). To examine the fidelity of synthetic Pfu DNA polymerase, products of natural PCR (1.2 kb D-DNA) after cycle 45 were purified by the V-elute Gel Mini Purification Kit (Beijing Zoman Biotech., Beijing, China) and cloned by Zero background ZT4 Simple-Blunt Fast Clone Kit (Beijing Zoman Biotech., Beijing, China) for Sanger sequencing, and calculated according to previously described methods.

### Example 2

### Total Chemical Synthesis of T7 RNA Polymerase and Uses Thereof

As discussed hereinabove, synthesizing the mirror-image version of an RNA polymerase, which uses double-stranded (ds) L-DNA templates, would enable the enzymatic transcription of all the mirror-image rRNAs and mRNAs needed for mirror-image translation. Hence, as another step in the proof of concept of some aspects of the present invention, both the natural (L-amino acids protein) and mirror-image versions of the 100 kDa T7 RNA polymerase, design of two split sites, was chemically synthesized.

The T7 RNA polymerase has known split forms, for example, Segall-Shapiro et al. [Mol Syst Biol., 2014, 30(10), pp. 742] used a transposon-based method to find several split sites in the T7 RNA polymerase. Tiyun Han et al. [ACS Synth Biol., 2017, 6(2), pp. 357-366.] designed photoactivatable genetic switches based on split T7 RNA polymerases to implement light-activated gene expression in different contexts. However, the split sites used in these natural enzymes are not always suitable for the chemical synthesis of T7 RNA polymerase: some of split sites of T7 RNA polymerase will significantly altering its enzymatic activity; some are near the N or C terminus of the protein peptide chain, resulting in one or more large protein fragment (more than 400-500 aa), which would still be too large to synthesize chemically.

In order to afford a practical domain-forming segments, a second split site was identified, using the criteria of low sequence conservation and structural flexibility, according to some embodiments of the present invention, which was not suggested hitherto, namely the split site between K363 and P364. The split site reported by Segall-Shapiro et al., between N601 and T602, as well as the split site (between K363 and P364) in the solvent-exposed loops of the structure of T7 RNA polymerase that was discovered while reducing the present invention to practice, together divided the polymerase into three fragments of roughly even lengths suitable for chemical synthesis (typically less than 400-500 aa): a 369-aa T7-split-N fragment (with a His6 tag added to the N terminus), a 238-aa T7-split-M fragment, and a 282-aa T7-split-C fragment, without significantly altering its enzymatic activity and fidelity. The above-mentioned split site can be selected to be near the above-mentioned sites in the same loop, namely from position 357 to position 366 and/or from position 564 to position 607. At the same time, the split T7 RNA polymerase can be used as a transcriptional AND-logic. For example, genetic switches in which the activity of T7 RNA polymerase is directly regulated by external signals are obtained with an engineering strategy of splitting the protein into fragments and using regulatory domains to modulate their reconstitutions. Robust switchable systems with excellent dark-off/light-on properties are obtained with the light-activatable VVD domain and its variants as regulatory domains.

The systematic isoleucine substitution approach was also implemented, based on a multiple sequence alignment (MSA) using T7-WT (SEQ ID No. 82), T7-371 (SEQ ID No. 83), YenP (SEQ ID No. 84), phiEap (SEQ ID No. 85), and KpnP (SEQ ID No. 86) polymerases, and structural information to mutate a number of isoleucines (14 out of 51, or 27% of Ile residues) in the T7 RNA polymerase into other amino acids such as valine, leucine, and methionine (I6V, I14L, I74V, I82V, I109V, I117L, I141V, I210M, I244L, I281V, I320V, I322L, I330V, I367L), without significantly altering its enzymatic activity and fidelity. This approach resulted in reducing the amino acid cost for the synthesis of this D-polymerase, which will facilitate its large-scale synthesis and practical application in the future.

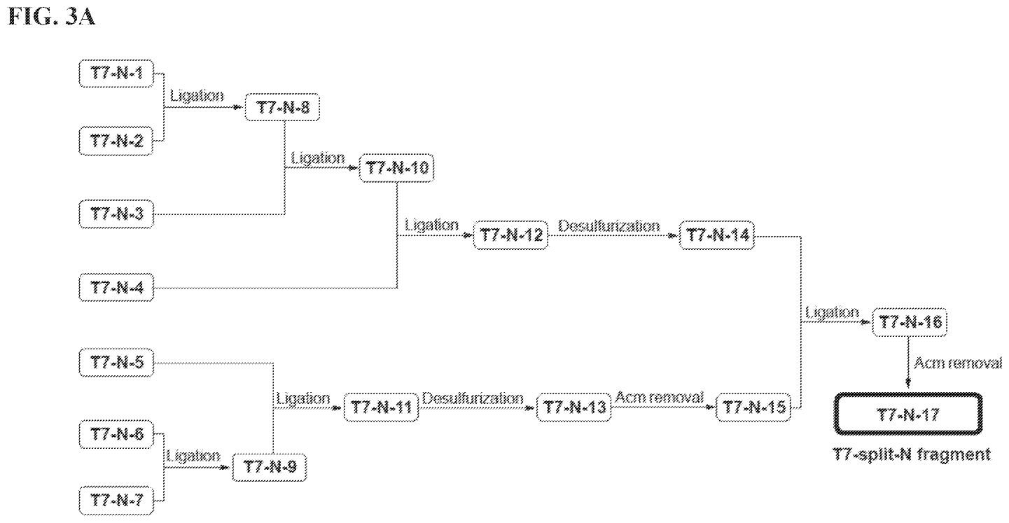

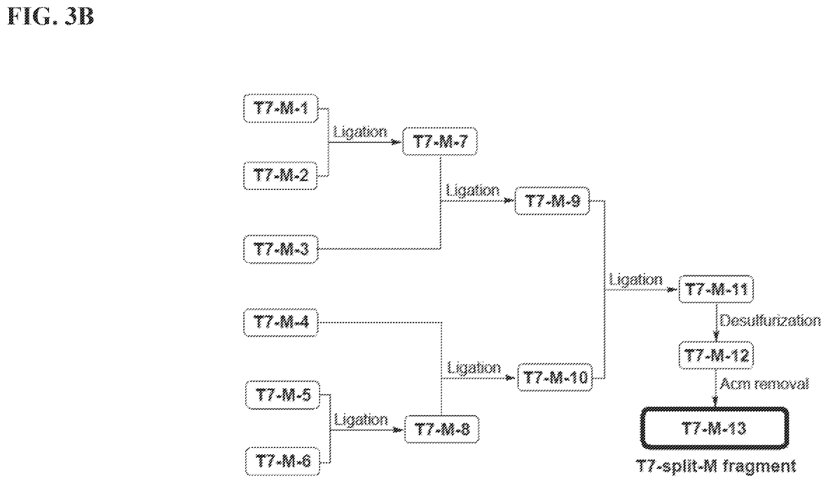

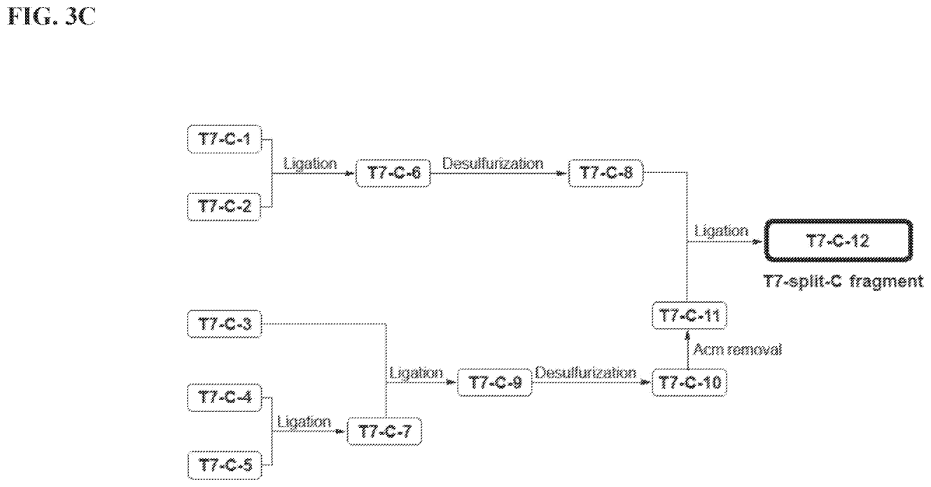

FIGS. 3A-C present the design flow of the synthetic route of the 369-aa mutant T7-split-N fragment (SEQ ID No. 87) (FIG. 3A), the 238-aa mutant T7-split-M fragment (SEQ ID No. 94) (FIG. 3B), and the 282-aa mutant T7-split-C fragment (SEQ ID No. 101) (FIG. 3C), including replacement of isoleucine residues, new NCL and a new split site between K363 and P364, which were introduced to facilitate protein synthesis in SPPS and ligation process, and reduce synthesis cost of the mirror-image version.

The total chemical synthesis of the T7 RNA polymerase was further carried out by introducing ligation-conducive residue replacements. The T7-split-N fragment was divided into 7 peptide segments ranging from 32 to 76 aa in lengths (SEQ ID Nos. 88-94), and the T7-split-M fragment was divided into 6 peptide segments ranging from 23 to 45 aa in lengths (SEQ ID Nos. 96-101), and the T7-split-C fragment was divided into 5 peptide segments ranging from 41 to 75 aa in lengths (SEQ ID Nos. 103-107). The peptide segments were prepared by Fmoc-based SPPS, purified by reversed-phase high-performance liquid chromatography (RP-HPLC), and assembled by hydrazide-based NCL with a convergent assembly strategy, followed by metal-free radical-based desulfurization. After the synthesis, ligation, purification, and lyophilization, about 3 mg of the T7-split-N fragment were obtained with an observed molecular weight (M.W.) of 41369.0 Da (calculated M.W. 41372.6 Da), about 2.5 mg T7-split-M fragment with an M.W. of 26786.0 Da (calculated M.W. 26787.4 Da), and about 4.8 mg T7-split-C fragment with an M.W. of 31459.0 Da (calculated M.W. 31459.9 Da) for the L-polymerase, about 9 mg of the D-T7-split-N fragment were obtained with an observed molecular weight (M.W.) of 41373.0 Da, about 8 mg T7-split-M fragment with an M.W. of 26787.0 Da, and about 15 mg T7-split-C fragment with an M.W. of 31459.0 Da for the D-polymerase.

### Folding of Synthetic Polymerases In Vitro:

The synthetic polymerase was folded by successive dialysis, followed by ultrafiltration to precipitate the impurities.

Lyophilized synthetic N, M and C fragments of T7 RNA polymerase were dissolved in a denaturation buffer containing 6 M Gn·HCl and 20 mM DTT, respectively. Protein folding was performed by mixing the N, M and C fragments equally (0.5 nmol/ml), and dialyzing against a renaturation buffer (50 mM Tris-HCl, 100 mM KCl, 10% glycerol, 1 mM EDTA, 10 mM DTT, pH 8.0) at 4° C. for 24 h with gentle stirring. After renaturation, the enzyme was dialyzed against a storage buffer containing 50% glycerol, 50 mM Tris-HCl (pH 8.0), 100 mM NaCl, 1 mM EDTA, 0.1% Triton X-100, 10 mM DTT at 4° C. for 12 h with gentle stirring, followed by ultrafiltration using an Amicon Utra centrifugal filter (0.5 ml, 100,000 MWCO).

### Transcription Activity and Fidelity of Synthetic T7 RNA Polymerase:

The natural and mirror-image transcriptions were performed in 10 μl reaction system containing 1× T7 reaction buffer (New England Biolabs, Beijing, China), with 500 μM (each) rNTPs, 10% DMSO, 5 mM DTT, template, and polymerase. To quantify the transcription activity of T7 RNA polymerase and its mutants, the polymerases were adjusted to the same concentration with wild-type (WT) T7 RNA polymerase by 12% SDS-PAGE (results not shown). The reactions were incubated at 37° C. for various times. The transcription activities of the natural and mirror-image T7 RNA polymerases showed that the polymerase can successfully transcribe the 160-bp DNA template (SEQ ID No. 108) and 1.5-kb DNA template (SEQ ID No. 109), indicating a wide length range of L-RNA molecules can be produced from the 1.5-kb L-DNA template by synthetic mirror-image T7 RNA polymerase (results not shown). A mixture of purified and concentration-determined single-stranded L-RNA transcripts of different lengths can be used as RNA marker (or RNA ladder) for RNA sizing and quantification on native or denaturing gels, which is superior to the commercial D-RNA merker (D-RNA ladder) since its resistance to natural RNase. The fidelity of the synthetic T7 RNA polymerase was also examined by reverse transcribing the DNase I-digested transcription product by Superscript IV high-fidelity reverse transcriptase, followed by PCR amplification by high-fidelity Pfu DNA polymerase, and sequencing the amplicons by Sanger sequencing, and measured an error rate (on the order of 10⁻⁶) consistent with the error rate of WT T7 RNA polymerase reported in previous studies.

L-tRNA^{Ser} charging:

L-tDNA^{Ser} (SEQ ID No. 110) was assembled by a mutant version of mirror-image Dpo4 (D-Dpo4-5m). L-tRNA^{Ser} was transcribed by high-fidelity mirror-image T7 RNA polymerase, and the reaction system containing 1× T7 reaction buffer A (40 mM Tris-HCl, 25 mM MgCl₂, 1 mM spermidine, 2 mM DTT, pH 8.0), with 2 mM (each) L-rNTPs, 10% DMSO, 0.3 μM template, and 2 μM polymerase was incubated at 37° C. for overnight. The products were purified by denaturing PAGE with single nucleotide resolution, and the purified products were analyzed by 10% denaturing PAGE (results not shown). L-tRNA^{Ser} charging was performed in 25 mM HEPES-KOH (pH 7.5), 50 mM KCl, 2 μM L-tRNA^{Ser}, and 10 μM L-dFx. The reaction system was heated to 95° C. for 2 min and slowly cooled to room temperature for annealing. Then 100 mM MgCl₂ was added to the system and the reaction system was incubated at room temperature for 10 min, then at 4° C. for 10 min. Finally, 5 mM D-Ser-DBE was added to the system and the reaction system was incubated at 4° C. for 6 h. Ethanol precipitation was performed by adding 1/10 volume of 3 M NaOAc, and 2.5 volumes of ethanol, and incubated at −20° C. for overnight. The products were analyzed by 8% acid PAGE (results not shown).

L-16S rRNA Purification:

L-16S rDNA (SEQ ID No. 109) was assembled by high-fidelity mirror-image Pfu DNA polymerase. L-16S rRNA was transcribed by high-fidelity mirror-image T7 RNA polymerase, and the reaction system containing 1×T7 reaction buffer (New England Biolabs, Beijing, China), with 500 μM (each) L-rNTPs, 10% DMSO, 5 mM DTT, template, and polymerase was incubated at 37° C. for overnight. The transcription products were purified from 2% low melting points agarose gel (Amersco, U.S.) by β-Agarase digestion. The gel slice containing the RNA sample was equilibrated with 10 volumes of 1× β-Agarase buffer for 60 min at room temperature, then melted at 70° C. for 15 min, and cooled to 45° C. The melted agarose solution was incubated with 2 units of β-Agarase (New England Biolabs, Beijing, China) at 45° C. for 60 min, followed by being placed at −20° C. for 15 min and centrifuged for 15 min at 4° C. The supernatant was transferred to a new microcentrifuge tube for ethanol precipitation with 1/10 volume of 3 M NaOAc and 2.5 volumes of ethanol added, and incubated at −20° C. overnight. The purified products were analyzed by 3% agarose gel (results not shown).

### L-Guanine Sensor:

Molecular discrimination of the guanine sensor was demonstrated by following the specificity of D- and L-guanine sensors transcribed by synthetic L- and D-T7 RNA polymerases. L-guanine sensor DNA template (SEQ ID No. 111) was assembled by D-Dpo4-5m. L-guanine sensor was transcribed by high-fidelity mirror-image T7 RNA polymerase, and the reaction system containing 1×T7 reaction buffer A (40 mM Tris-HCl, 25 mM MgCl₂, 1 mM spermidine, 2 mM DTT, pH 8.0), with 2 mM (each) L-rNTPs, 10% DMSO, 0.2 μM template, and 2 μM polymerase was incubated at 37° C. for overnight. The products were purified by polyacrylamide gel in 8 M urea, and the purified products were analyzed by 10% denaturing PAGE (results not shown). 1 μM L-guanine sensor and 10 μM DFHBI was incubated at 37° C. in a buffer containing 40 mM HEPES (pH 7.4), 125 mM KCl and 1 mM MgCl₂. 1 mM guanine was then rapidly added to the solutions and fluorescence emission was recorded over a 15 min period under continuous illumination at 37° C. using the following instrumental parameters: excitation wavelength, 460 nm; emission wavelength, 500 nm; slit widths, 12 nm. 0.1 μM RNA and 10 μM DFHBI were incubated with 100 μM guanine or competing molecules and assayed for fluorescence emission at 500 nm. The guanine sensor saturates at 100 μM guanine, and showed a high level of molecular discrimination against GTP and adenine at the same concentrations (results not shown).

### L-38-6 RNA Polymerization Reactions:

The DNA template of L-38-6 ribozyme (SEQ ID No. 112) and L-class I ligase DNA template (SEQ ID No. 113) was assembled by D-Dpo4-5m. The RNA were transcribed by high-fidelity mirror-image T7 RNA polymerase, and the reaction system containing 1× T7 reaction buffer A (40 mM Tris-HCl, 25 mM MgCl₂, 1 mM spermidine, 2 mM DTT, pH 8.0), with 2 mM (each) L-rNTPs, 10% DMSO, 0.3 μM template, and 2 μM polymerase was incubated at 37° C. for overnight. The products were purified by polyacrylamide gel in 8 M urea (results not shown). RNA polymerization reactions used 100 nM L-38-6 ribozyme (SEQ ID No. 114), 80 nM L-5′-FAM-labelled primer (SEQ ID No. 115), and 100 nM L-class I ligase template (SEQ ID No. 116). The RNAs were annealed by first being heated to 80° C. for 30 s then slowly cooled to 17° C., and then added to a reaction mixture containing 4 mM each L-rNTPs, 200 mM MgCl₂, 25 mM Tris·HCl pH 8.3, and 0.05% Tween-20, which was incubated at 17° C. for various periods of times. The products were concentrated by ssDNA/RNA Clean & Concentrator kit (ZYMO RESEARCH, CA, U.S.), and then mixed with a denaturation buffer (98% formamide, 0.25 mM EDTA) followed by being heated to 65° C. for 10 min, and then quickly placed on ice. The samples were separated by 10% polyacrylamide gel in 8 M urea and scanned by a Typhoon Trio+ system operated under Cy2 mode.

Kinetics of RNA Degradation in Natural and Mirror-Image 16S rRNA:

To evaluate the RNA integrity under controlled conditions, three prepared transcripts including natural 16S rRNA, natural 16S rRNA with RNase inhibitor and mirror-image 16S rRNA, were detected and resolved by Bioanalyzer method. Natural and mirror-image 16S rRNA were transcribed by natural and mirror-image T7 RNA polymerase, respectively, and purified from 2% low melting point agarose gel by β-Agarase I digestion. The purified RNA was placed at 37° C. for 5 min, 30 min, 1 h, 2 h, 4 h, 8 h, 18 h, 24 h, 48 h, 72 h, 7 d, 15 d, 30 d, 60 d, and 100 d, and the RNA quality was assessed on the basis of electropherogram images of microchip gel electrophoresis. Minimal signs of degradation of natural 16S rRNA were seen when placed for 30 minutes at 37° C., and the degradation was more pronounced at 1 hour with a substantial elevation of the baseline. After 6 hours at 37° C., the peaks disappear completely due to advanced degradation. In the samples of natural 16S rRNA with RNase inhibitor, minimal signs of degradation were seen when placed for 4 hours at 37° C., degradation of RNA was more pronounced at 8 hours with a substantial elevation of the baseline. After 48 hours at 37° C., the peaks disappear completely due to advanced degradation. In the samples of mirror-image 16S rRNA, no signs of degradation could be detected, even placed for 15 days at 37° C. This shows that RNA has stronger stability under the condition of complete elimination of RNase. Using L-RNA system to measure the hydrolysis kinetics of RNA under different conditions, can serve as a control to evaluate the effectiveness of RNase-inhibiting reagents.

### Example 3

### Mirror-Image DNA Information Storage

Once obtaining the high-fidelity mirror-image Pfu DNA polymerase, a proof of concept of mirror-image DNA information storage, according to some embodiments of the present invention, was carried out by exploring its application in mirror-image DNA information storage through the faithful writing and reading of L-DNA sequences.

The below paragraph from the 1860 publication by Louis Pasteur in which the concept of mirror-image molecules and mirror-image biology systems was first proposed, was encoded into DNA sequences (see, Table 4), and archived into 11 L-DNA segments of 220 bp in lengths (Table 5), each assembled from 4 short, synthetic L-DNA oligos of 70-90 nt.

Pasteur: “And consequently, if the mysterious influence to which the asymmetry of natural products is due should change its sense or direction, the constitutive elements of all living beings would assume the opposite asymmetry. Perhaps a new world would present itself to our view. Who could foresee the organisation of living things if cellulose, right as it is, became left; if the albumen of the blood, now left, became right? These are mysteries which furnish much work for the future, and demand henceforth the most serious consideration from science.”

TABLE 4

| Character | Code | Character | Code |
|---|---|---|---|
| a | ACG | space | ATC |
| b | GTA | , | TCC |
| c | CAG | . | TCT |
| d | TGC | 0 | ATT |
| e | ATG | 1 | ACA |
| f | CTA | 2 | ACC |
| g | GAT | 3 | AGA |
| h | TCG | 4 | AGG |
| i | AGC | 5 | TAA |
| j | AAT | 6 | TAT |
| k | GCA | 7 | TTA |
| l | TGA | 8 | TTC |
| m | CTG | 9 | TTG |
| n | TAC | - | TGT |
| o | AGT | ? | TGG |
| p | GAC | : | CAA |
| q | AAC | ; | CAC |
| r | TCA | ! | CTT |
| s | TAG | * | CTC |
| t | ACT | / | CCA |
| u | CAT | /n | CCT |
| v | GTC | ° | CCG |
| w | CGA | ′ | CGC |
| x | GCT | ″ | CGG |
| y | CGT | ( | GAA |
| z | AAG | ) | GAG |
| \hat{ } | ATA | | |

Information-storing double-stranded L-DNA segments of 220 bp, each assembled by the mirror-image Pfu DNA polymerase using mirror-image assembly PCR from 4 short, synthetic L-DNA oligos of 70-90 nt, and the L-DNA storage library containing all 11 segments (L-library), were analyzed by 2.5% agarose gel electrophoresis and stained by ExRed. M, DNA marker (results not shown), and listed in Table 5. Table 5 presents the sequences used for L-DNA information storage, wherein lowercase letters are M13-F and M13-R sequences for amplification, and underlined (underscore; understrike) letters are unique sequences for sequencing individual segments.

TABLE 5

| Segments | Sequence |
|---|---|
| DNA storage-S1 | 5′-gtaaaacgacggccagtTCGCGCGTTTC |
| (SEQ ID No. 1) | GGTGATGACGGTGAAAACCATTACAATAACG |
| | TACTGCATCCAGAGTTACTAGATGAACCATA |
| | TGTACACTTGACGTTCCATCAGCCTAATCAC |
| | TTCGATGATCCTGCGTTAGACTATGTCAAGC |
| | AGTCATTAGATCAGCTACCTATGACATATGT |
| | ACCAGATGATCACTAGTATCgtcatagctgt |
| | ttcctg-3′ |
| DNA storage-S2 | 5′-gtaaaacgacggccagtTCTGACACATG |
| (SEQ ID No. 2) | CAGCTCCCGGAGACGGTCAATTACCCGATCG |
| | AGCCAGTCGATCACTTCGATGATCACGTAGC |
| | GTCTGCTGATGACTTCACGTATCAGTCTAAT |
| | CTACACGACTCATTCAACGTGAATCGACTCA |
| | AGTTGCCATCAGACTTAGATCAGCTAGATCT |
| | GCCATATGATCTAGTCGAGTgtcatagctgt |
| | ttcctg-3′ |
| DNA storage-S3 | 5′-gtaaaacgacggccagtCAGCTTGTCTG |
| (SEQ ID No. 3) | TAAGCGGATGCCGGGAGCAATTAGACATTGA |
| | TGCATCCAGTCGACGTACGATATGATCAGCA |
| | CTTAGATCTAGATGTACTAGATGATCAGTTC |
| | AATCTGCAGCTCAATGCAGACTAGCAGTTAC |
| | TCCATCACTTCGATGATCCAGAGTTACTAGA |
| | CTAGCACTCATACTAGCGTCgtcatagctgt |
| | ttcctg-3′ |
| DNA storage-S4 | 5′-gtaaaacgacggccagtGACAAGCCCGT |
| (SEQ ID No. 4) | CAGGGCGCGTCAGCGGGTCATTAGGATGATC |
| | ATGTGAATGCTGATGTACACTTAGATCAGTC |
| | TAATCACGTGATGAATCTGAAGCGTCAGCTA |
| | CGATATCGTAATGAGCTACGATTAGATCCGA |
| | AGTCATTGATGCATCACGTAGTAGCATCTGA |
| | TGATCACTTCGATGATCAGTgtcatagctgt |
| | ttcctg-3′ |
| DNA storage-S5 | 5′-gtaaaacgacggccagtTTGGCGGGTGT |
| (SEQ ID No. 5) | CGGGGCTGGCTTAACTATGATTTAAGACGAC |
| | AGTTAGAGCACTATGATCACGTAGCGTCTGC |
| | TGATGACTTCACGTTCTATCATAGACATGTC |
| | ATCGACGGACTAGATCACGATCTACATGCGA |
| | ATCCGAAGTTCATGATGCATCCGAAGTCATT |
| | GATGCATCGACTCAATGTAGgtcatagctgt |
| | ttcctg-3′ |
| DNA storage-S6 | 5′-gtaaaacgacggccagtCGGCATCAGAG |
| (SEQ ID No. 6) | CAGATTGTACTGAGAGTGCATTTATATGTAC |
| | ACTATCAGCACTTAGATGTGACTAATCACTA |
| | GTATCAGTCATTCAATCGTCAGCATGCGATC |
| | TATCATACGATCGAGTATCCAGAGTCATTGA |
| | TGCATCCTAAGTTCAATGTAGATGATGATCA |
| | CTTCGATGATCAGTTCAGATgtcatagctgt |
| | ttcctg-3′ |
| DNA storage-S7 | 5′-gtaaaacgacggccagtACCATATGCGG |
| (SEQ ID No. 7) | TGTGAAATACCGCACAGATATTTTAACGTAC |
| | AGCTAGACGACTAGCAGTTACATCAGTCTAA |
| | TCTGAAGCGTCAGCTACGATATCACTTCGAG |
| | CTACGATTAGATCAGCCTAATCCAGATGTGA |
| | TGACATTGAAGTTAGATGTCCATCTCAAGCG |
| | ATTCGACTATCACGTAGATCgtcatagctgt |
| | ttcctg-3′ |
| DNA storage-S8 | 5′-gtaaaacgacggccagtGCGTAAGGAGA |
| (SEQ ID No. 8) | AAATACCGCATCAGGCGTGATTTTGAGCACT |
| | ATCAGCTAGTCCATCGTAATGCAGACGCTGA |
| | TGATCTGAATGCTAACTCACATCAGCCTAAT |
| | CACTTCGATGATCACGTGAGTACATCTGATG |
| | TACATCAGTCTAATCACTTCGATGATCGTAT |
| | GAAGTAGTTGCTCCATCTACgtcatagctgt |
| | ttcctg-3′ |
| DNA storage-S9 | 5′-gtaaaacgacggccagtATTCGCCATTC |
| (SEQ ID No. 9) | AGGCTGCGCAACTGTTGGGATTTTGAGTCGA |
| | ATCTGAATGCTAACTTCCATCGTAATGCAGA |
| | CGCTGATGATCTCAAGCGATTCGACTTGGAT |
| | CATAACTTCGATGTAGATGATCACGTCAATG |
| | ATCCTGCGTTAGACTATGTCAAGCATGTAGA |
| | TCCGATCGAGCCAGTCGATCgtcatagctgt |
| | ttcctg-3′ |
| DNA storage-S10 | 5′-gtaaaacgacggccagtAAGGGCGATCG |
| (SEQ ID No. 10) | GTGCGGGCCTCTTCGCTATACAATTCTACAT |
| | TCATACAGCTAGTCGATCCTGCATCAGTCGA |
| | TCCGAAGTTCAGCAATCCTAAGTTCAATCAC |
| | TTCGATGATCCTACATACTCATTCAATGTCC |
| | ATCACGTACTGCATCTGCATGCTGACGTACT |
| | GCATCTCGATGTACCAGATGgtcatagctgt |
| | ttcctg-3′ |
| DNA storage-S11 | 5′-gtaaaacgacggccagtTACGCCAGCTG |
| (SEQ ID No. 11) | GCGAAAGGGGGATGTGCTGACAACACTAAGT |
| | TCAACTTCGATCACTTCGATGATCCTGAGTT |
| | AGACTATCTAGATGTCAAGCAGTCATTAGAT |
| | CCAGAGTTACTAGAGCTGCATGTCAACGACT |
| | AGCAGTTACATCCTATCAAGTCTGATCTAGC |
| | AGAGCATGTACCAGATGTCTgtcatagctgt |
| | ttcctg-3′ |
| DNA barcode | 5′-gtaaaacgacggccagtATATGAAGTAC |
| (SEQ ID No. 12) | TCATTAGATCATAGACAGTTACTGCTCCATC |
| | ATAGTAATGAGCAATAGCTACGATgtcatag |
| | ctgtttcctg-3′ |

The reading of L-DNA can be achieved through sequencing-by-synthesis using the mirror-image Pfu DNA polymerase by the phosphorothioate approach (with L-deoxynucleoside α-thiotriphosphates (L-dNTPaSs), and cleavage by 2-iodoethanol), or using the mutant mirror-image Pfu DNA polymerase by the chain-termination approach with L-dideoxynucleoside triphosphates (L-ddNTPs). A bi-directional sequencing approach was also applied using 5′-labelled primers with two different dyes (FAM and Cy5, respectively), which improved the maximum read length in a single reaction to about 180 bp by denaturing polyacrylamide gel electrophoresis (PAGE; PCR amplification). The information-bearing L-DNA 203 bp sequences in the storage medium were each amplified by D-Dpo4-5m from the DNase I-treated L-DNA storage library with segment-specific sequencing primers, analyzed by 2.5% agarose gel electrophoresis and stained by ExRed. M, DNA marker (results not shown), and the L-DNA storage segment S1 (SEQ ID No. 1) was sequenced using mirror-image DNA polymerase by phosphorothioate approach to retrieve the encoded digital data. Specifically, the L-DNA Si segment was specifically amplified with 5′-FAM-labelled (forward) and 5′-Cy5-labelled (reverse) sequencing primers by D-Dpo4-5m in 4 separate PCR reactions, within which one of the L-dNTPs was replaced by the corresponding L-dNTPαS, each cleaved by 2-iodoethanol, and analyzed by 10% denaturing PAGE and scanned by a Typhoon Trio+ system operated under Cy2 and Cy5 mode. Sequencing chromatograms of the information-storing L-DNA segment S1 by D-Dpo4-5m with L-dNTPaSs and 5′-labelled forward and reverse sequencing primers were processed by ImageJ software (results not shown). Although the mirror-image Pfu DNA polymerase is able to amplify and sequence the L-DNA storage segment, D-Dpo4 was used in the actual experiment for its convenient synthesis.

### Chiral Steganography:

Steganography is known as the art and science of hiding messages such that none other than the recipient can see them or know of their existence. This is in contrast to cryptography, where the existence of the information itself is not hidden, but only its content. The L-DNA information storage system provided herein can also be applied to secure communication through designing a chiral steganography experiment, in which a D-DNA storage library encoding Louis Pasteur's 1860 paragraph serves as a “cover text”, and an L-DNA key helps to decrypt the “stego text” (secret message). To make the secret message even more disguised, a chimeric D-DNA/L-DNA key molecule (SEQ ID No. 46) was designed to convey either a false message “error” or a secret message “mirror” depending on the chirality of reading. D-DNA storage library was sequenced by Sanger sequencing to retrieve the “cover text”. Using natural PCR one can only amplify and sequence the D-DNA part of the chimeric key embedded in the storage library, revealing the false message, whereas using mirror-image PCR one can amplify and sequence the L-DNA part of the chimeric key, revealing the secret message. Steganography and cryptography are two prominent techniques to keep data secret. Steganography is the art of concealing the existence of a secret message while cryptography refers to the practice of converting a secret message into an unreadable format. The chiral steganography developed here is potential to be combined with DNA cryptography to provide an extra layer of security using encrypted data.

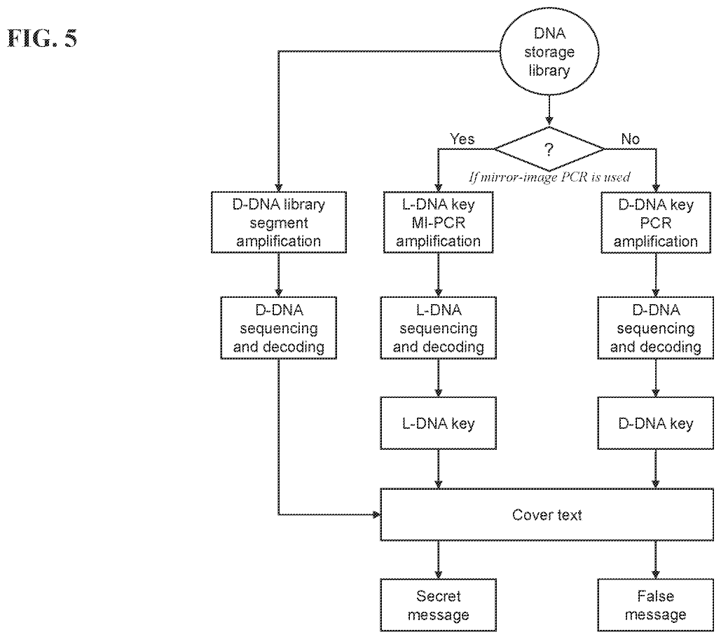

FIG. 5 presents a flowchart illustrating DNA based steganography, according to some embodiments of the present invention, embedding a chimeric D-DNA/L-DNA key molecule in a seemingly ordinary D-DNA storage library to convey a secret message.

To demonstrate the abilities of L-DNA information storage medium to evade biological degradation and contamination from natural environments, fresh water samples were collected from a local pond and added a trace amount of 100-bp L-DNA barcode (SEQ ID No. 12) (50 μg/L, or 770 pM) encoding the location information of sample collection (“Lotus Pond, Beijing”) (Table 5) to the collected water samples. Remarkably, the message-carrying L-DNA barcode remained stable and amplifiable for up to 7 months (an arbitrarily chosen time period) and potentially beyond. In comparison, D-DNA barcode of the same sequence and concentration was not amplifiable after merely a day. Specifically, the amplification of D-DNA barcode after 24 h by L-Dpo4-5m and amplification of L-DNA barcode after 1 year by D-Dpo4-5m was followed by agarose gel electrophoresis, wherein PCR amplification of D-DNA barcode was effected by L-Dpo4-5m in 40-ml pond water samples after 24 hours, and MI-PCR amplification of L-DNA barcode was effected by D-Dpo4-5m in 40-ml pond water samples after 1 year, analyzed by 3% sieving agarose gel electrophoresis and stained by ExRed. M, DNA marker (results not shown).

Furthermore, L-DNA barcoding of the microbial DNA extracted from the water samples was also bioorthogonal in that it was specifically amplifiable by mirror-image PCR with D-polymerase and L-DNA primers, and did not affect the D-DNA metagenomic microbial sequencing results.

Encouraged by the faithful writing and reading of L-DNA sequences, the assembly of a full-length 1.5-kb mirror-image bacterial 16S rRNA gene was carried out by the high-fidelity mirror-image Pfu DNA polymerase. The attempt began by testing the gene assembly using synthetic L-polymerase on D-DNA using a two-step assembly procedure: DNA blocks of 450-600 bp were first assembled from short, synthetic oligos of about 90 nt (Table 6), followed by a second step to assemble the DNA blocks into a full-length 16S rRNA gene (SEQ ID No. 81).

TABLE 6

| Primer | Sequence |
|---|---|
| TT16S-F1 | 5′-TTTGTTGGAGAGTTTGATCCTGGCTCA |
| (SEQ ID No. 13) | GGGTGAACGCTGGCGGCGTGCCTAAGACAT |
| | GCAAGTCGTGCGGGCCGCGGGGTTTTACTC |
| | CGT-3′ |
| TT16S-R1 | 5′-TTTCCCCGGGTTGTCCCCCTCTTCCGG |
| (SEQ ID No. 14) | GTAGGTCACCCACGCGTTACTCACCCGTCC |
| | GCCGCTGACCACGGAGTAAAACCCCGCGGC |
| | CCG-3′ |
| TT16S-F2 | 5′-GGAAGAGGGGGACAACCCGGGGAAACT |
| (SEQ ID No. 15) | CGGGCTAATCCCCCATGTGGACCCGCCCCT |
| | TGGGGTGTGTCCAAAGGGCTTTGCCCGCTT |
| | CCG-3′ |
| TT16S-R2 | 5′-CGGCTACCCGTCGTCGCCTTGGTGGGC |
| (SEQ ID No. 16) | CATTACCCCACCAACTAGCTGATGGGACGC |
| | GGGCCCATCCGGAAGCGGGCAAAGCCCTTT |
| | GGA-3′ |
| TT16S-F3 | 5′-AAGGCGACGACGGGTAGCCGGTCTGAG |
| (SEQ ID No. 17) | AGGATGGCCGGCCACAGGGGCACTGAGACA |
| | CGGGCCCCACTCCTACGGGAGGCAGCAGTT |
| | AGG-3′ |
| TT16S-R3 | 5′-ACCCCGAAGGGCTTCTTCCTCCAAGCG |
| (SEQ ID No. 18) | GCGTCGCTCCGTCAGGCTTGCGCCCATTGC |
| | GGAAGATTCCTAACTGCTGCCTCCCGTAGG |
| | AGT-3′ |
| TT16S-F4 | 5′-CTTGGAGGAAGAAGCCCTTCGGGGTGT |
| (SEQ ID No. 19) | AAACTCCTGAACCCGGGACGAAACCCCCGA |
| | CGAGGGGACTGACGGTACCGGGGTAATAGC |
| | GCC-3′ |
| TT16S-R4 | 5′-ACGCCCAGTGAATCCGGGTAACGCTCG |
| (SEQ ID No. 20) | CGCCCTCCGTATTACCGCGGCTGCTGGCAC |
| | GGAGTTGGCCGGCGCTATTACCCCGGTACC |
| | GTC-3′ |
| TT16S-F5 | 5′-GCGTTACCCGGATTCACTGGGCGTAAA |
| (SEQ ID No. 21) | GGGCGTGTAGGCGGCCTGGGGCGTCCCATG |
| | TGAAAGACCACGGCTCAACCGTGGGGGAGC |
| | GTG-3′ |
| TT16S-R5 | 5′-TATCTGCGCATTTCACCGCTACTCCGG |
| (SEQ ID No. 22) | GAATTCCACCACCCTCTCCCACCGTCTAGC |
| | CTGAGCGTATCCCACGCTCCCCCACGGTTG |
| | AGC-3′ |
| TT16S-F6 | 5′-AATTCCCGGAGTAGCGGTGAAATGCGC |
| (SEQ ID No. 23) | AGATACCGGGAGGAACGCCGATGGCGAAGG |
| | CAGCCACCTGGTCCACCCGTGACGCTGAGG |
| | CGC-3′ |
| TT16S-R6 | 5′-AGACCTAGCGCGCATCGTTTAGGGCGT |
| (SEQ ID No. 24) | GGACTACCCGGGTATCTAATCCGGTTTGCT |
| | CCCCACGCTTTCGCGCCTCAGCGTCACGGG |
| | TGG-3′ |
| TT16S-F7 | 5′-CCCTAAACGATGCGCGCTAGGTCTCTG |
| (SEQ ID No. 25) | GGTCTCCTGGGGGCCGAAGCTAACGCGTTA |
| | AGCGCGCCGCCTGGGGAGTACGGCCGCAAG |
| | GCT-3′ |
| TT16S-R7 | 5′-TTCGCGTTGCTTCGAATTAAACCACAT |
| (SEQ ID No. 26) | GCTCCACCGCTTGTGCGGGCCCCCGTCAAT |
| | TCCTTTGAGTTTCAGCCTTGCGGCCGTACT |
| | CCC-3′ |
| TT16S-F8 | 5′-GGAGCATGTGGTTTAATTCGAAGCAAC |
| (SEQ ID No. 27) | GCGAAGAACCTTACCAGGCCTTGACATGCT |
| | AGGGAACCCGGGTGAAAGCCTGGGGTGCCC |
| | CGC-3′ |
| TT16S-R8 | 5′-GGACTTAACCCAACACCTCACGGCACG |
| (SEQ ID No. 28) | AGCTGACGACGGCCATGCAGCACCTGTGCT |
| | AGGGCTCCCCTCGCGGGGCACCCCAGGCTT |
| | TCA-3′ |
| TT16S-F9 | 5′-CGTGCCGTGAGGTGTTGGGTTAAGTCC |
| (SEQ ID No. 29) | CGCAACGAGCGCAACCCCCGCCGTTAGTTG |
| | CCAGCGGTTCGGCCGGGCACTCTAACGGGA |
| | CTG-3′ |
| TT16S-R9 | 5′-TGTGTCGCCCAGGCCGTAAGGGCCATG |
| (SEQ ID No. 30) | CTGACCAGACGTCGTCCCCTCCTTCCTCCC |
| | GCTTTCGCGGGCAGTCCCGTTAGAGTGCCC |
| | GGC-3′ |
| TT16S-F10 | 5′-GGCCCTTACGGCCTGGGCGACACACGT |
| (SEQ ID No. 31) | GCTACAATGCCCACTACAAAGCGATGCCAC |
| | CCGGCAACGGGGAGCTAATCGCAAAAAGGT |
| | GGG-3′ |
| TT16S-R10 | 5′-GATCCGCGATTACTAGCGATTCCGGCT |
| (SEQ ID No. 32) | TCATGGGGTCGGGTTGCAGACCCCAATCCG |
| | AACTGGGCCCACCTTTTTGCGATTAGCTCC |
| | CCG-3′ |
| TT16S-F11 | 5′-GCCGGAATCGCTAGTAATCGCGGATCA |
| (SEQ ID No. 33) | GCCATGCCGCGGTGAATACGTTCCCGGGCC |
| | TTGTACACACCGCCCGTCACGCCATGGGAG |
| | CGG-3′ |
| TT16S-R11 | 5′-CGACTTCGCCCCAGTCACGGGCCCTAC |
| (SEQ ID No. 34) | CCTCGGCGCCTGCCCGTAGGCTCCCGGCGA |
| | CTTCGGGTAGAGCCCGCTCCCATGGCGTGA |
| | CGG-3′ |
| TT16S-R12 | 5′-CCGCACCTTCCGGTACAGCTACCTTGT |
| (SEQ ID No. 35) | TACGACTTCGCCCCAGTCACGGGCCCT-3′ |
| M13-F | 5′-GTAAAACGACGGCCAGT-3′ |
| (SEQ ID No. 36) | |
| M13-R | 5′-CAGGAAACAGCTATGAC-3′ |
| (SEQ ID No. 37) | |

In an initial attempt, Sanger sequencing of the full-length D-DNA product indicated that only about 40% of the assembled sequences were correct (Table 3), with most of the errors being nucleotide deletions, likely arising from the minus 1- and 2-nt products from oligo synthesis. Hence, the oligo purification approach was modified using denaturing PAGE with single nucleotide resolution to substantially improve the quality of the synthetic oligos by removing the majority of the minus 1- and 2-nt products, after which most of the deletion errors were eliminated, and about 90% of the final assembled sequences were correct (the rest of them contained only single randomly occurred mutations). Therefore, using the same oligo purification approach and mirror-image assembly PCR, the assembly of a full-length 1.5-kb mirror-image 16S rRNA gene was performed, which will be a template for the future enzymatic transcription into mirror-image 16S rRNA, a linchpin in building a functional mirror-image ribosome. Specifically, the mirror-image 16S rRNA gene assembled by the mirror-image Pfu DNA polymerase was followed by agarose gel electrophoresis, wherein full-length 1.5-kb mirror-image bacterial 16S rRNA gene obtained by mirror-image assembly PCR using mirror-image Pfu DNA polymerase, analyzed by 1.5% agarose gel electrophoresis and stained by ExRed. M, DNA marker (results not shown).

### DNA-Templated RNA Polymerization:

RNA polymerization was performed in 1× Thermopol buffer (New England Biolabs, MA, U.S.), 3 mM MgSO₄, 0.625 mM (each) NTPs, 0.5 μM 5′-FAM-labelled DNA primer (21 nt), and 1 μM ssDNA template (41 nt), and polymerase. Prior to the addition of polymerase, the reaction system was heated to 94° C. for 30 s and slowly cooled to 4° C. for annealing. Primer extension reaction took place at 65° C. for 10 min. The reaction was stopped by the addition of loading buffer containing 98% formamide, 0.25 mM EDTA, and 0.0125% SDS, and the products were analyzed by 20% denaturing PAGE in 8 M urea. Specifically, DNA-templated RNA polymerization activity assay of different mutant Pfu DNA polymerases was followed by PAGE analysis, wherein DNA-template-directed primer extension by different Pfu DNA polymerase mutants with 41-nt single-stranded DNA template, 5′-FAM-labelled 21-nt DNA primer, and NTPs, incubated for 10 min at 65° C. and analyzed by 20% PAGE in 8 M urea (results not shown).

### Writing and Reading L-DNA:

A paragraph from the 1860 publication by Louis Pasteur containing 550 characters (see text above) was converted into DNA sequences with 1650 nucleotides (Table 4) and encoded into 11 L-DNA segments of 220 bp in lengths (Table 5), each assembled from 4 short, synthetic L-DNA oligos of 70-90 nt. The assembly PCR program settings were 94° C. for 3 min (initial denaturation); 94° C. for 30 s, 55° C. for 30 s, and 72° C. for 1 min (depending on the amplicon length), for 35 cycles; 72° C. for 10 min (final extension). For phosphorothioate approach, L-DNA segment was amplified with 5′-FAM-labelled (forward) and 5′-Cy5-labelled (reverse) primers by D-Dpo4-5m (a mutant version of Dpo4 to facilitate its chemical synthesis) in four separated PCR reactions, within each of which one of the L-dNTPs was replaced by the corresponding L-dNTPαS. The PCR program settings were 86° C. for 3 min (initial denaturation); 86° C. for 30 s, 54° C. (Tm-dependent) for 1 min, and 65° C. for 1-2.5 min (depending on the amplicon length), for 45 cycles; 65° C. for 5 min (final extension). The PCR products (mixed 1:20 w/w with unlabeled carrier dsDNA of the same length) were purified by 8% PAGE and dissolved in water to a concentration of about 200 ng/μl. For each sequencing reaction, 2.5 μl of double-labelled L-DNA was mixed with 2.5 μl of a denaturation buffer (98% formamide, 0.25 mM EDTA) containing 2% (v/v) 2-iodoethanol, followed by being heated to 95° C. for 3 min, and then quickly placed on ice. For chain-termination approach, L-DNA segment was amplified with 5′-FAM-labelled (forward) and/or 5′-Cy5-labelled (reverse) primers by the mirror-image Pfu DNA polymerase mutant (D215A, L490W) (SEQ ID No. 77) in four separated PCR reactions, within each of which one of the L-dNTPs was replaced by the corresponding L-ddNTP in a certain proportion. The PCR program settings were 94° C. for 3 min (initial denaturation); 94° C. for 30 s, 54° C. (Tm-dependent) for 30 s, and 72° C. for 30-60 s (depending on the amplicon length), for 20 cycles; 72° C. for 5 min (final extension). The double-labelled PCR products were each mixed with an equal volume of a denaturation buffer (98% formamide, 0.25 mM EDTA), followed by being heated to 95° C. for 3 min, and then quickly placed on ice. The sequencing gel of D-DNA segment S1 by chain-termination approach using expressed Pfu DNA polymerase mutant (D215A, L490W) with ddNTPs and 5′-Cy5-labelled (reverse) sequencing primersmplification products of D-DNA segment S1 by Pfu DNA polymerase mutant (D215A, L490W) with ddNTPs and 5′-Cy5-labelled reverse sequencing primer, were analyzed by 10% denaturing PAGE and scanned by a Typhoon Trio+ system operated under Cy5 mode. A, dATP partially replaced by ddATP; C, dCTP partially replaced by ddCTP; G, dGTP partially replaced by ddGTP; T, dTTP partially replaced by ddTTP (results not shown). The sequencing samples were loaded on slabs of 0.4 mm×340 mm×300 mm, separated by 10% polyacrylamide gel in 8 M urea. The gel was pre-run at 50 W (constant power) for 2 h until being heated to 30-40° C. After loading, the gel was run at 50 W (constant power) for 1.5 h and paused for fluorescent scanning, following which the gel went on running and was scanned every other hour until the total running time was up to 5 h. The polyacrylamide gel was scanned by a Typhoon Trio′ system operated Cy2 and Cy5 modes, respectively. Gel quantitation and chromatogram analysis were performed by the ImageJ software.

### Chiral Steganography:

The chimeric D-DNA/L-DNA oligos were synthesized with D- and L-deoxynucleoside phosphoramidites using the methods described above. The oligos D-F1, D-R1, D/L-F2 and D/L-R2 (Table 7) were heated to 95° C. for 3 min and slowly cooled to 4° C. for annealing, and the annealed double-stranded DNAs were ligated by the T3 DNA ligase (New England Biolabs, MA, U.S.) at 25° C. for 1.5 h. The D-DNA storage library served as a “cover text” was prepared by the TransStart FastPfu Fly polymerase (TransGen Biotech., Beijing, China) using similar methods as for L-DNA storage library. The chimeric double-stranded D-DNA/L-DNA key purified by agarose gel was added to the D-DNA storage library at 1:1 concentration ratio as each D-DNA segment. The 11 information-storing D-DNA segments and the D-DNA part of the chimeric key were each amplified with segment-specific primers from the storage library and cloned by Zero Background ZT4 Simple-Blunt Fast Clone Kit (Beijing Zoman Biotech., Beijing, China) for Sanger sequencing (Supplementary Table S6). The L-DNA part of the chimeric key was amplified with L-M13F and L-M13R primers by D-Dpo4-5m from the storage library, and sequenced by the phosphorothioate approach.

Table 7 presents the sequences used for chiral steganography, wherein lowercase letters are D-DNA sequences, uppercase letters are L-DNA sequences, and underlined (underscore; understrike) letters are unique sequences for amplification and sequencing individual segments.

TABLE 7

| Oligo | Sequence |
|---|---|
| D-F1 | 5′-gtgctgcaaggcgattaattaggtatac |
| (SEQ ID No. 38) | aaccagaaccagattaagattgtata-3′ |
| D-R1 | 5′-ctatgactgttaacctatacaatcttaa |
| (SEQ ID No. 39) | tctggttctggttgtatacctaattaatcgc |
| | cttgcagcac-3′ |
| D/L-F2 | 5′-ggttaacagtcatagctgtttcctgGTA |
| (SEQ ID No. 40) | AAACGACGGCCAGTATTACCTTAACAACCTA |
| | TACCACATATACCAGGTTCAGATTCTATAGG |
| | TTCACAGTCATAGCTGTTTCCTG-3′ |
| D/L-R2 | 5′-CAGGAAACAGCTATGACTGTGAACCTAT |
| (SEQ ID No. 41) | AGAATCTGAACCTGGTATATGTGGTATAGGT |
| | TGTTAAGGTAATACTGGCCGTCGTTTTACca |
| | ggaaacag-3′ |
| D-DNA key-F | 5′-gtgctgcaaggcgatta-3′ |
| (SEQ ID No. 42) | |
| D-DNA key-R | 5′-caggaaacagctatgac-3′ |
| (SEQ ID No. 43) | |
| L-DNA key-F | 5′-GTAAAACGACGGCCAGT-3′ |
| (SEQ ID No. 44) | |
| L-DNA key-R | 5′-CAGGAAACAGCTATGAC-3′ |
| (SEQ ID No. 45) | |
| Chimeric D- | 5′-gtgctgcaaggcgattaattaggtatac |
| DNA/L-DNA key | aaccagaaccagattaagattgtataggtta |
| (SEQ ID No. 46) | acagtcatagctgtttcctgGTAAAACGACG |
| | GCCAGTATTACCTTAACAACCTATACCACAT |
| | ATACCAGGTTCAGATTCTATAGGTTCACAGT |
| | CATAGCTGTTTCCTG-3′ |

### L-DNA Barcoding:

Unpurified environmental water samples were collected from the Lotus Pond at Tsinghua University (40° 0′27″N, 116° 19′34″E) on Dec. 8, 2019. Synthetic D- and L-DNA oligos were heated to 95° C. for 5 min and slowly cooled to 4° C. for annealing, and the annealed dsDNA were added to the water samples to a concentration of 50 μg/L. To amplify the DNA barcodes (SEQ ID No. 12), 2 ml of water sample was filtered by 0.22 μm filter (Pall Corporation, WI, U.S.), resuspended in DEPC-treated water by an Amicon Utra centrifugal filter unit (0.5 ml, 10,000 MWCO), before being amplified by D-/L-Pfu DNA polymerases. The PCR program settings were 94° C. for 3 min (initial denaturation); 94° C. for 30 s, 55° C. for 30 s, and 72° C. for 1 min for 25 cycles; 72° C. for 10 min (final extension). For metagenomic microbial DNA extraction, the water samples were filtered with a 0.2-μm Supor 200 PES Membrane Disc Filter (Pall, NY, U.S.), and microbial DNA was extracted by the DNeasy PowerSoil Kit (Qiagen, MD, U.S.).

16S rRNA Gene Assembly:

Synthetic oligos of about 90 nt in lengths at concentrations of 0.005-0.02 μM each (inner) or 0.2 μM each (outer) were assembled into full-length gene in two steps. In the first step, the assembly PCR program settings were 94° C. for 3 min (initial denaturation); 94° C. for 30 s, 60° C. for 30 s, and 72° C. for 3 min for 35 cycles; 72° C. for 10 min (final extension). In the second step, the previously assembled DNA blocks at about 450-550 bp in lengths were purified by 1.5% agarose gel before being subject to assembly PCR. The assembly PCR program settings were 94° C. for 3 min (initial denaturation); 94° C. for 30 s, 60° C. for 30 s, and 72° C. for 7 min for 35 cycles; 72° C. for 10 min (final extension). The assembled product was further amplified with PCR program settings: 94° C. for 3 min (initial denaturation); 94° C. for 30 s, 60° C. for 30 s, and 72° C. for 7 min for 35 cycles; 72° C. for 10 min (final extension). The final D-DNA products (SEQ ID No. 81) of natural assembly PCR purified by the V-elute Gel Mini Purification Kit (Beijing Zoman Biotech., Beijing, China), and cloned by Zero Background ZT4 Simple-Blunt Fast Clone Kit (Beijing Zoman Biotech., Beijing, China) for Sanger sequencing.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. L. Ceze, J. Nivala, K. Strauss, Molecular digital data storage using DNA. Nat Rev Genet 20, 456-466 (2019).
2. N. Goldman et al., Towards practical, high-capacity, low-maintenance information storage in synthesized DNA. Nature 494, 77-80 (2013).
3. G. M. Church, Y. Gao, S. Kosuri, Next-generation digital information storage in DNA. Science 337, 1628 (2012).
4. L. Pasteur, Researches on the Molecular Asymmetry of Natural Organic Products. Soc. Chim. Paris, (1860).
5. Z. Wang, W. Xu, L. Liu, T. F. Zhu, A synthetic molecular system capable of mirror-image genetic replication and transcription. Nature Chemistry 8, 698-704 (2016).
6. M. Peplow, A Conversation with Ting Zhu. ACS Cent Sci 4, 783-784 (2018).
7. M. Peplow, Mirror-image enzyme copies looking-glass DNA. Nature 533, 303-304 (2016).
8. S. L. Beaucage, M. H. Caruthers, Deoxynucleoside Phosphoramidites—a New Class of Key Intermediates for Deoxypolynucleotide Synthesis. Tetrahedron Lett 22, 1859-1862 (1981).
9. Y. Liu et al., Synthesis and applications of RNAs with position-selective labelling and mosaic composition. Nature 522, 368-372 (2015).
10. R. B. Merrifield, Solid Phase Peptide Synthesis 0.1. Synthesis of a Tetrapeptide. Journal of the American Chemical Society 85, 2149-& (1963).
11. L. Z. Yan, P. E. Dawson, Synthesis of peptides and proteins without cysteine residues by native chemical ligation combined with desulfurization. J Am Chem Soc 123, 526-533 (2001).
12. P. Dawson, T. Muir, I. Clark-Lewis, S. Kent, Synthesis of proteins by native chemical ligation. Science 266, 776-779 (1994).
13. G.-M. Fang et al., Protein Chemical Synthesis by Ligation of Peptide Hydrazides. Angewandte Chemie International Edition 50, 7645-7649 (2011).
14. R. Milton, S. Milton, S. Kent, Total chemical synthesis of a D-enzyme: the enantiomers of HIV-1 protease show reciprocal chiral substrate specificity. Science 256, 1445-1448 (1992).
15. A. A. Vinogradov, E. D. Evans, B. L. Pentelute, Total synthesis and biochemical characterization of mirror image barnase. Chemical Science 6, 2997-3002 (2015).
16. M. T. Weinstock, M. T. Jacobsen, M. S. Kay, Synthesis and folding of a mirror-image enzyme reveals ambidextrous chaperone activity. Proceedings of the National Academy of Sciences of the United States of America 111, 11679-11684 (2014).
17. W. Xu et al., Total chemical synthesis of a thermostable enzyme capable of polymerase chain reaction. Cell discovery 3, 17008 (2017).
18. W. Jiang et al., Mirror-image polymerase chain reaction. Cell discovery 3, 17037 (2017).
19. A. Pech et al., A thermostable d-polymerase for mirror-image PCR. Nucleic Acids Res 45, 3997-4005 (2017).
20. L. E. Zawadzke, J. M. Berg, A Racemic Protein. Journal of the American Chemical Society 114, 4002-4003 (1992).
21. M. Wang et al., Mirror-image gene transcription and reverse transcription. Chem 5, 848-857 (2019).
22. B. J. Lamarche, S. Kumar, M. D. Tsai, ASFV DNA polymerse X is extremely error-prone under diverse assay conditions and within multiple DNA sequence contexts. Biochemistry 45, 14826-14833 (2006).
23. H. Ling, F. Boudsocq, R. Woodgate, W. Yang, Crystal structure of a Y-family DNA polymerase in action: a mechanism for error-prone and lesion-bypass replication. Cell 107, 91-102 (2001).
24. F. Boudsocq, S. Iwai, F. Hanaoka, R. Woodgate, Sulfolobus solfataricus P2 DNA polymerase IV (Dpo4): an archaeal DinB-like DNA polymerase with lesion-bypass properties akin to eukaryotic polη. Nucleic Acids Research 29, 4607-4616 (2001).
25. J. Cline, J. C. Braman, H. H. Hogrefe, PCR fidelity of pfu DNA polymerase and other thermostable DNA polymerases. Nucleic Acids Res 24, 3546-3551 (1996).
26. C. J. Hansen, L. Wu, J. D. Fox, B. Arezi, H. H. Hogrefe, Engineered split in Pfu DNA polymerase fingers domain improves incorporation of nucleotide gamma-phosphate derivative. Nucleic Acids Res 39, 1801-1810 (2011).
27. Q. Wan, S. J. Danishefsky, Free-radical-based, specific desulfurization of cysteine: a powerful advance in the synthesis of polypeptides and glycopolypeptides. Angew Chem Int Ed Engl 46, 9248-9252 (2007).
28. J. T. Hyde C, Owen D, Quibell M, Sheppard R C., Some ‘difficult sequences’ made easy. International journal of peptide and Protein Research 43, 431-440 (1994).
29. T. Johnson, M. Quibell, R. C. Sheppard, N,O-bisFmoc derivatives of N-(2-hydroxy-4-methoxybenzyl)-amino acids: Useful intermediates in peptide synthesis. Journal of Peptide Science 1, 11-25 (1995).
30. J. S. Zheng et al., Robust Chemical Synthesis of Membrane Proteins through a General Method of Removable Backbone Modification. J Am Chem Soc 138, 3553-3561 (2016).
31. M. T. Jacobsen et al., A Helping Hand to Overcome Solubility Challenges in Chemical Protein Synthesis. J Am Chem Soc 138, 11775-11782 (2016).
32. F. W. Torsten Wöhr, Adel Nefzi, Barbara Rohwedder, Tatsunori Sato, Xicheng Sun, Manfred Mutter, Pseudo-Prolines as a Solubilizing, Structure-Disrupting Protection Technique in Peptide Synthesis. J Am Chem Soc 118, 9218-9227 (1996).
33. M. K. Pascal Dumy, Declan E. Ryan, Barbara Rohwedder, Torsten Wöhr, Manfred Mutter, Pseudo-Prolines as a Molecular Hinge: Reversible Induction of cis Amide Bonds into Peptide Backbones. J. Am. Chem. Soc. 119, 918-925 (1997).
34. Y. Sohma et al., ‘O-Acyl isopeptide method’ for the efficient synthesis of difficult sequence-containing peptides: use of ‘O-acyl isodipeptide unit’. Tetrahedron Letters 47, 3013-3017 (2006).
35. I. Coin, The depsipeptide method for solid-phase synthesis of difficult peptides. Journal of peptide science: an official publication of the European Peptide Society 16, 223-230 (2010).
36. G. M. Fang, J. X. Wang, L. Liu, Convergent chemical synthesis of proteins by ligation of peptide hydrazides. Angew Chem Int Ed Engl 51, 10347-10350 (2012).
37. J. S. Zheng, S. Tang, Y. K. Qi, Z. P. Wang, L. Liu, Chemical synthesis of proteins using peptide hydrazides as thioester surrogates. Nat Protoc 8, 2483-2495 (2013).
38. N. K. L., G. Gerald, E. Fritz, V. Hans-Peter, Direct sequencing of polymerase chain reaction amplified DNA fragments through the incorporation of deoxynucleoside α-thiotriphosphates. Nucleic Acids Research, 21 (1988).
39. G. Gish, F. Eckstein, DNA and RNA sequence determination based on phosphorothioate chemistry. Science 240, 1520-1522 (1988).
40. C. Y. Chen, DNA polymerases drive DNA sequencing-by-synthesis technologies: both past and present. Front Microbiol 5, 305 (2014).
41. A. S. Xiong et al., A simple, rapid, high-fidelity and cost-effective PCR-based two-step DNA synthesis method for long gene sequences. Nucleic Acids Res 32, e98 (2004).
42. A. Tiessen, P. Perez-Rodriguez, L. J. Delaye-Arredondo, Mathematical modeling and comparison of protein size distribution in different plant, animal, fungal and microbial species reveals a negative correlation between protein size and protein number, thus providing insight into the evolution of proteomes. BMC Res Notes 5, 85 (2012).
43. C. Cozens, V. B. Pinheiro, A. Vaisman, R. Woodgate, P. Holliger, A short adaptive path from DNA to RNA polymerases. Proc Natl Acad Sci USA 109, 8067-8072 (2012).
44. X. Liu, T. F. Zhu, Sequencing mirror-Image DNA chemically. Cell Chemical Biology 25, 1151-1156 e1153 (2018).
45. D. Wade et al., All-D amino acid-containing channel-forming antibiotic peptides. Proc Natl Acad Sci USA 87, 4761-4765 (1990).

1. A method of chemically producing a protein, comprising ligating at least two ligation-conducive segments of the protein, wherein each of said ligation-conducive segments is chemically-synthesizable, and obtainable by:
i. identifying at least one ligation-conducive sequence in the amino-acid sequence of the protein, parsing said amino-acid sequence of the protein at said ligation-conducive sequence to thereby obtain a plurality of ligation-conducive segments; and
ii. if each of said ligation-conducive segments is chemically-synthesizable, chemically synthesizing each of said ligation-conducive segments;
iii. if any one of said ligation-conducive segments is not chemically-synthesizable, identifying at least one structurally-lose section in said ligation-conducive segment, substituting at least one amino acid in said structurally-lose section with a ligation-conducive amino acid residue so as to introduce a ligation-conducive sequence in said structurally-lose section, parsing the amino-acid sequence of the protein at said ligation-conducive sequence; and chemically synthesizing each of said ligation-conducive segments,
wherein in Step (i), at least one of said ligation-conducive sequences is in a structurally-lose section in the protein.

2-3. (canceled)

4. The method of claim 1, further comprising, prior to Step (i),
a) splitting said amino-acid sequence of the protein into at least two domain-forming segments;
b) if each of said domain-forming segments is chemically-synthesizable, chemically synthesizing each of said domain-forming segments; and
c) co-folding said domain-forming segments to thereby obtain the protein.

5. (canceled)

6. The method of claim 4, wherein if one of said domain-forming segments is not chemically-synthesizable,
d) identifying at least one ligation-conducive sequence in said domain-forming segment, and parsing the amino-acid sequence of said domain-forming segment at said ligation-conducive sequence to thereby obtain a plurality of chemically-synthesizable ligation-conducive segments;
e) if said domain-forming segment is essentially devoid of a ligation-conducive sequence, or any one of said ligation-conducive segments is not chemically-synthesizable, identifying at least one structurally-lose section in said domain-forming segment or said ligation-conducive segment;
f) substituting at least one amino acid in said structurally-lose section or said ligation-conducive segment with a ligation-conducive amino acid residue so as to introduce a ligation-conducive sequence in said structurally-lose section or said ligation-conducive segment, and parsing the amino-acid sequence of said domain-forming segment at said ligation-conducive sequence to thereby obtain a plurality of sequences of chemically-synthesizable ligation-conducive segments; and
g) chemically synthesizing each of said chemically-synthesizable ligation-conducive segments.

7-9. (canceled)

10. The method of claim 1, wherein the protein comprises at least 240 amino-acid residues.

11-12. (canceled)

13. The method of claim 1, wherein the protein is produced using at least 90% non-Gly D-amino-acid residues, and having essentially a mirror-imaged 3D structure compared to a 3D structure of a corresponding biologically produced protein.

14. (canceled)

15. The method of claim 13, further comprising, substituting at least one Ile residue with a D-amino-acid residue selected from the group consisting of a D-Ala residue, a D-Val residue, a D-Leu residue, a D-Thr residue, a D-Phe residue, a D-Met residue, a Gly residue, and a D-Pro residue.

16. A protein, prepared according to the method of claim 1, wherein the protein is at least about 240 amino-acid residues long.

17-19. (canceled)

20. The protein of claim 16, being an RNA polymerase, capable of synthesizing RNA from ribonucleotides using a DNA template.

21. The protein of claim 20, wherein said RNA polymerase is a T7 RNA polymerase, or a Pfu DNA polymerase mutant.

22. (canceled)

23. The protein of claim 16, being a DNA polymerase, capable of synthesizing DNA from deoxyribonucleotides.

24. The protein of claim 23, wherein said DNA polymerase is a Pfu DNA polymerase.

25. A method of chemically producing a D-amino acids protein, comprising ligating at least two ligation-conducive segments of the D-amino acids protein, wherein each of said ligation-conducive segments comprises at least 90% non-Gly D-amino-acid residues and is chemically-synthesizable, and obtainable by:
i. identifying at least one ligation-conducive sequence in the amino-acid sequence of a corresponding L-amino-acid protein, parsing said amino-acid sequence at said ligation-conducive sequence to thereby obtain a plurality of ligation-conducive segments; and;
ii. if each of said ligation-conducive segments is chemically-synthesizable, chemically synthesizing each of said ligation-conducive segments using at least 90% non-Gly D-amino-acid residues;
iii. if any one of said ligation-conducive segments is not chemically-synthesizable, identifying at least one structurally-lose section in said ligation-conducive segment, substituting at least one amino acid in said structurally-lose section with a ligation-conducive amino acid residue so as to introduce a ligation-conducive sequence in said structurally-lose section, parsing the amino-acid sequence of said ligation-conducive segment at said ligation-conducive sequence; and chemically synthesizing each of said ligation-conducive segments using at least 90% non-Gly D-amino-acid residue,
wherein in Step (i), at least one of said ligation-conducive sequences is in a structurally-lose section in said corresponding L-amino-acid protein.

26-27. (canceled)

28. The method of claim 25, further comprising, prior to Step (i),
a) splitting said amino-acid sequence of said L-amino-acid protein into at least two domain-forming segments;
b) if each of said domain-forming segments is chemically-synthesizable, chemically synthesizing each of said domain-forming segments using at least 90% non-Gly D-amino-acid residues; and
c) co-folding said domain-forming segments, thereby obtaining the D-amino acids protein.

29. The method of claim 28, wherein if one of said domain-forming segments is not chemically-synthesizable,
d) identifying at least one ligation-conducive sequence in said domain-forming segment, and parsing the amino-acid sequence of said domain-forming segment at said ligation-conducive sequence to thereby obtain a plurality of chemically-synthesizable ligation-conducive segments;
e) if said domain-forming segment is essentially devoid of a ligation-conducive sequence, or any one of said ligation-conducive segments is not chemically-synthesizable, identifying at least one structurally-lose section in said domain-forming segment or said ligation-conducive segment;
f) substituting at least one amino acid in said structurally-lose section or said ligation-conducive segment with a ligation-conducive amino acid residue so as to introduce a ligation-conducive sequence in said structurally-lose section or said ligation-conducive segment, and parsing the amino-acid sequence of said domain-forming segment at said ligation-conducive sequence; and
g) chemically synthesizing each of said ligation-conducive segments using at least 90% non-Gly D-amino-acid residues thereby obtaining said domain-forming segment.

30-32. (canceled)

33. The method of claim 25, wherein the D-amino acids protein comprises at least 240 amino-acid residues.

34-37. (canceled)

38. A D-amino acids protein, prepared according to the method of claim 25.

39-62. (canceled)

63. A process of producing an L-polydeoxyribonucleic acid molecule enzymatically, comprising:
providing a D-amino acids DNA polymerase prepared according to the method of claim 25, and capable of synthesizing L-DNA from L-deoxyribonucleotides; and
reacting said D-amino acids DNA polymerase with a template L-DNA molecule, L-DNA primers and a plurality of L-deoxyribonucleotides,
to thereby enzymatically producing the L-DNA molecule.

64. The process of claim 63, wherein said D-amino acids DNA polymerase is a Pfu DNA polymerase.

65. The process of claim 64, wherein said Pfu DNA polymerase is essentially as provided herein.

66. A process of producing an L-polyribonucleic acid (L-RNA) molecule enzymatically, comprising:
providing a D-amino acids RNA polymerase prepared according to the method of claim 25, and capable of synthesizing L-RNA from L-ribonucleotides; and
reacting said D-amino acids RNA polymerase with a template L-DNA molecule, L-DNA/RNA primers and a plurality of L-ribonucleotides,
to thereby enzymatically producing the L-RNA molecule.

67-97. (canceled)
